(19) United States
(12) Patent Application Publication
(10) Pub. No.: US 2003/0219459 A1
(43) Pub. Date: Nov. 27, 2003

(54) Prion protein carrier-conjugates

(75) Inventors: Martin Bachmann, Seuzach (CH); Patrik Maurer, Winterthur (CH); Erica Pellicioli, Au (CH); Wolfgang A. Renner, Kilchberg (CH)

Correspondence Address: STERNE, KESSLER, GOLDSTEIN & FOX P.L.L.C., Suite 600, 1100 New York Avenue, N.W., Washington, DC, 20005-3934, US

(73) Assignee: CYTOS BIOTECHNOLOGY AG

(21) Appl. No.: 10/346,190

(22) Filed: Jan. 17, 2003

Related U.S. Application Data
(63) Continuation-in-part of application No. 10/050,902, filed on Jan. 18, 2002.
(63) Continuation-in-part of application No. PCT/IB02/00166, filed on Jan. 21, 2002.
(60) Provisional application No. 60/396,590, filed on Jul. 18, 2002.
(60) Provisional application No. 60/393,725, filed on Jul. 8, 2002.

(51) Int. Cl. A61K 48/00; A61K 39/00; A01N 63/00; A61K 39/12

(52) U.S. Cl. 424/199.1; 424/185.1; 424/93.2

(57) ABSTRACT

The present invention is related to the fields of molecular biology, virology, immunology and medicine. The invention provides a composition comprising an ordered and repetitive antigen or antigenic determinant array, and in particular a prion peptide or prion protein-VLP-array. More specifically, the invention provides a composition comprising a virus-like particle and at least one prion protein (PrP) or a dimer thereof, or a PrP peptide bound thereto. The invention also provides a process for producing the conjugates and the ordered and repetitive arrays, respectively. The compositions of the invention are useful in the production of vaccines for the treatment of prion diseases and as a pharmaccine to prevent or cure prion diseases and to efficiently induce immune responses, in particular antibody responses. Furthermore, the compositions of the invention are particularly useful to efficiently induce self-specific immune responses within the indicated context.

### CROSS REFERENCE TO RELATED APPLICATIONS

This application claims priority benefit of U.S. Provisional Application No. 60/396,590, filed Jul. 18, 2002, U.S. Provisional Application No. 60/393,725, filed Jul. 8, 2002, and U.S. Provisional Application No. 60/389,898, filed Jun. 20, 2002. The present application also claims priority under 35 U.S.C §120 to International Application PCT/IB02/00166, filed Jan. 21, 2002, which designated the United States and was published in English under PCT article 21(2) as WO 02/056905, and U.S. application Ser. No. 10/050,902, filed Jan. 18, 2002. All above applications are incorporated herein by reference in their entireties.

### BACKGROUND OF THE INVENTION

1. Field of the Invention

The present invention is related to the fields of molecular biology, virology, immunology and medicine. The invention provides a composition comprising an ordered and repetitive antigen or antigenic determinant array, and in particular a prion peptide or prion protein-array. More specifically, the invention provides a composition comprising a virus-like particle and at least one prion protein (PrP) or a dimer thereof, or a PrP peptide bound thereto. The invention also provides a process for producing the conjugates and the ordered and repetitive arrays, respectively. The compositions of the invention are useful in the production of vaccines for the treatment of prion diseases and as a pharmaccine to prevent or cure prion diseases and to efficiently induce immune responses, in particular antibody responses. Furthermore, the compositions of the invention are particularly useful to efficiently induce self-specific immune responses within the indicated context.

2. Related Art

Prion diseases are an increasing threat for society. Specifically, prion-induced BSE (Bovine Spongiform Encephalopathy) in cattle represents a disease that has long been neglected and may affect a great number of animals throughout Europe. Moreover, a variant form of CJD (Creutzfeldt-Jakob-Disease) is attributed to infection of humans after consumption of meat of prion-infected cattle. Although the number of infected people has been relatively low so far, it seems possible that the disease may become epidemic. However, long-term prognosis for the development of vCJD may be particular difficult, since incubation times between infection and overt disease are very long and estimated to be 10 years or more.

Prions are cellular proteins existing in most mammalian species. Prion proteins (PrP) exist in two forms, a normally folded form that is usually present in healthy individuals (PrP^{C}) and a misfolded form that is associated with prion diseases (Prp^{Sc}). The current prion hypotheses postulate that the misfolded prion form Prp^{Sc} can catalyze the refolding of healthy prion PrP^{C} into disease causing Prp^{Sc} (Aguzzi, A and Weissmann, C., Nature 389:795-798 (1997)). In some rare instances, this transition may also occur spontaneously, causing classical CJD in humans. Some mutations in PrP^{C} are associated with an increase in this spontaneous transition, causing the various forms of familial CJD. However, Prp^{Sc} may also be infectious and may be transmitted by blood transfusion or via the food chain. The latter form of prion mediated disease is known as Kuru and used to occur in human cannibals. However, since species that are feeding on their own individuals are not abundant, this form of orally transmitted disease was too rare to be documented for other species.

The massive feeding of cows with meat-and-bone meal throughout Europe now changed the situation and the number of cows infected with a transmissible form of BSE-causing Prp^{Sc} dramatically increased in recent years, afflicting hundreds of thousands of cows. This sudden appearance of massive numbers of BSE-diseased cows caused great fear in the human population that a similar disease may be induced in humans. Indeed, in 1996, the first case of a variant form of CJD was reported that could be attributed to the consumption of Prp^{Sc} infected beef. Until now, this fear has further increased, since the number of infected humans has constantly increased during the following years and no cure is in sight.

Experimentally, it is possible that BSE-like diseases may occur also in other species. The mechanism of prion transmission has been studied in great detail. It is now clear that prions first replicate in the lymphoid organs of infected mice and are subsequently transported to the central nervous system. Follicular dendritic cells (FDCs), a rare cell population in lymphoid organs, seems to be essential for both replication of prion proteins in the lymphoid organs and transport into the central nervous system (Brandner, S., et al., Science 288:1257-9(2000)). FDCs are a poorly studied cell type but it is now clear that they depend upon the production of lymphotoxin and/or TNF by B cells for their development (Mackay, F., and Browning, J. L., Nature 395:26-27 (1998)). Indeed, mice deficient for lymphotoxin do not exhibit FDCs (Matsumoto, M. S., et al., Science 264:703-707 (1996)). Moreover, they fail to be productively infected with prions and do not succumb to disease.

Recently, a transgenic mouse model expressing an anti-prion protein antibody of the IgM isotype has been generated (Heppner,. F. L., et al., Science 294:178-182 (2001). Transgenic expression of anti-PrP antibodies prevented accumulation of PrP^{Sc} and infectivity in spleens of transgenic mice after intraperitoneal prion inoculation, indicating that extracerebral pathogenesis was arrested. In addition, no PrP^{Sc} was detectable in brains 234 days after intraperitoneal prion inoculation.

Due to the urgent need to develop diagnostic methods suitable for mass screening of prion infected blood or tissues as well as to develop passive and active immunization strategies against prion diseases, a lot of effort has been undertaken to identify immunogenic regions and epitopes, respectively, of the PrP^{Sc}. In WO 93/11155 a large number of synthetic polypeptides having at least one antigenic site of a prion protein are described together with methods for their manufacture as well as antibodies raised against such polypeptides. Furthermore, conjugates of some of the described peptides to ovalbumin and the generation of antiserum raised against these conjugates in rabbits are reported. In WO 99/15651 synthetic polypeptides containing one or more defined sequences of PrP, and, in particular, identified epitopes of PrP are described. Further peptides comprising a YYX (YYR, YYD, or YYQ) amino acid sequence suggested to be continuous epitopes of PrP and having antigenicity as a PrP^{Sc} as well as the coupling of the peptides to keyhole limpet hemocyanin are described in WO 00/78344.

However, so far there has been little evidence that vaccines might be effective for protection against prion diseases, in particular, since it is usually difficult to induce antibody responses to self-molecules by conventional vaccination.

One way to improve the efficiency of vaccination is to increase the degree of repetitiveness of the antigen applied. Unlike isolated proteins, viruses induce prompt and efficient immune responses in the absence of any adjuvants both with and without T-cell help (Bachmann and Zinkernagel, Ann. Rev. Immunol. 15:235-270 (1991)). Although viruses often consist of few proteins, they are able to trigger much stronger immune responses than their isolated components. For B-cell responses, it is known that one crucial factor for the immunogenicity of viruses is the repetitiveness and order of surface epitopes. Many viruses exhibit a quasi-crystalline surface that displays a regular array of epitopes which efficiently crosslinks epitope-specific immunoglobulins on B cells (Bachmann and Zinkernagel, Immunol. Today 17:553-558 (1996)). This crosslinking of surface immunoglobulins on B cells is a strong activation signal that directly induces cell-cycle progression and the production of IgM antibodies. Further, such triggered B cells are able to activate T helper cells, which in turn induce a switch from IgM to IgG antibody production in B cells and the generation of long-lived B cell memory—the goal of any vaccination (Bachmann and Zinkernagel, Ann. Rev. Immunol. 15:235-270 (1997)). Viral structure is even linked to the generation of anti-antibodies in autoimmune disease and as a part of the natural response to pathogens (see Fehr, T., et al., J Exp. Med. 185:1785-1792 (1997)). Thus, antibodies presented by a highly organized viral surface are able to induce strong anti-antibody responses.

As indicated, however, the immune system usually fails to produce antibodies against self-derived structures. For soluble antigens present at low concentrations, this is due to tolerance at the Th cell level. Under these conditions, coupling the self-antigen to a carrier that can deliver T help may break tolerance. For soluble proteins present at high concentrations or membrane proteins at low concentration, B and Th cells may be tolerant. However, B cell tolerance may be reversible (anergy) and can be broken by administration of the antigen in a highly organized fashion coupled to a foreign carrier (Bachmann and Zinkernagel, Ann. Rev. Immunol. 15:235-270 (1997)).

### BRIEF SUMMARY OF THE INVENTION

We have now found that prion proteins or dimers thereof, or prion peptides, which are bound to a core particle having a structure with an inherent repetitive organization, and hereby in particular to virus-like-particles (VLP's) and subunits of VLP's, respectively, leading to highly ordered and repetitive conjugates represent potent immunogens for the induction of antibodies specific for PrP. Therefore, the present invention provides a therapeutic mean for the treatment of prion diseases, which is based on an ordered and repetitive PrP-core particle array, and in particular a VLP-PrP-conjugate and -array, respectively. This therapeutic is able to induce high titers of anti-PrP antibodies in a vaccinated animal.

The present invention, thus, provides for a composition comprising: (a) a core particle with at least one first attachment site; and (b) at least one antigen or antigenic determinant with at least one second attachment site, wherein said antigen or antigenic determinant is a prion protein (PrP) or a dimer thereof, or a prion peptide, and wherein said second attachment site being selected from the group consisting of (i) an attachment site not naturally occurring with said antigen or antigenic determinant; and (ii) an attachment site naturally occurring with said antigen or antigenic determinant, wherein said second attachment site is capable of association to said first attachment site; and wherein said antigen or antigenic determinant and said core particle interact through said association to form an ordered and repetitive antigen array. Preferred embodiments of core particles suitable for use in the present invention are a virus, a virus-like particle, a bacteriophage, a bacterial pilus or flagella or any other core particle having an inherent repetitive structure capable of forming an ordered and repetitive antigen array in accordance with the present invention.

More specifically, the invention provides a composition comprising an ordered and repetitive antigen or antigenic determinant array, and hereby in particular prion protein or a dimer thereof, or prion peptide VLP conjugates. More specifically, the invention provides a composition comprising a virus-like particle and at least one prion protein (PrP) or a dimer thereof, or a PrP peptide bound thereto. The invention also provides a process for producing the conjugates and the ordered and repetitive arrays, respectively. The compositions of the invention are useful in the production of vaccines for the treatment of prion diseases and as a pharmaccine to prevent or cure prion diseases and to efficiently induce immune responses, in particular antibody responses. Furthermore, the compositions of the invention are particularly useful to efficiently induce self-specific immune responses within the indicated context.

In the present invention, a prion protein or a dimer thereof, or a prion peptide is bound to a core particle and VLP, respectively, typically in an oriented manner, yielding an ordered and repetitive prion protein or prion peptide antigen array. Furthermore, the highly repetitive and organized structure of the core particles and VLPs, respectively, mediates the display of the prion protein or a dimer thereof, or prion peptide in a highly ordered and repetitive fashion leading to a highly organized and repetitive antigen array. Furthermore, binding of the prion protein or a dimer thereof, or prion peptide to the core particle and VLP, respectively, provides T helper cell epitopes, since the core particle and VLP is foreign to the host immunized with the core particle-prion protein/peptide array and VLP-prion protein/peptide array, respectively. Those arrays differ from prior art conjugates in their highly organized structure, dimensions, and in the repetitiveness of the antigen on the surface of the array.

In one aspect of the invention, the prion protein or prion peptide is expressed in a suitable expression host compatible with proper folding of the prion protein, or synthesized, while the core particle and the VLP, repespectively, is expressed and purified from an expression host suitable for the folding and assembly of the core particle and the VLP, repespectively. Prion peptides may be chemically synthesized. The prion protein, prion protein domain or prion peptide array is then assembled by binding the prion protein, prion protein domain or prion peptide to the core particle and the VLP, respectively.

In another aspect, the present invention provides for a composition comprising (a) a virus-like particle, and (b) at least one antigen or antigenic determinant, wherein said antigen or said antigenic determinant is a prion protein (PrP) or a dimer thereof, or a PrP peptide, and wherein said at least one antigen or antigenic determinant is bound to said virus-like particle.

In a further aspect, the present invention provides for a pharmaceutical composition comprising (a) the composition of claim 1 or claim 22, and (b) an acceptable pharmaceutical carrier.

In still a further aspect, the present invention provides for a vaccine composition comprising a composition comprising: (a) a core particle with at least one first attachment site; and (b) at least one antigen or antigenic determinant with at least one second attachment site, wherein said antigen or antigenic determinant is a prion protein (PrP) or a dimer thereof, or a prion peptide, and wherein said second attachment site being selected from the group consisting of (i) an attachment site not naturally occurring with said antigen or antigenic determinant; and (ii) an attachment site naturally occurring with said antigen or antigenic determinant, wherein said second attachment site is capable of association to said first attachment site; and wherein said antigen or antigenic determinant and said core particle interact through said association to form an ordered and repetitive antigen array.

In a further aspect, the present invention provides for a vaccine composition comprising a composition, wherein said composition comprising (a) a virus-like particle; and (b) at least one antigen or antigenic determinant, wherein said antigen or said antigenic determinant is a prion protein (PrP) or a dimer thereof, or a PrP peptide; and wherein said at least one antigen or antigenic determinant is bound to said virus-like particle.

In still a further aspect, the present invention provides for a process for producing a composition of claim 1 comprising (a) providing a virus-like particle; and (b) providing at least one antigen or antigenic determinant, wherein said antigen or said antigenic determinant is a prion protein (PrP) or a dimer thereof, or a PrP peptide; (c) combining said virus-like particle and said at least one antigen or antigenic determinant so that said at least one antigen or antigenic determinant is bound to said virus-like particle.

In still a further aspect, ;the present invention provides a process for producing a composition of claim 22 comprising: (a) providing a core particle with at least one first attachment site; (b) providing at least one antigen or antigenic determinant with at least one second attachment site, wherein said antigen or antigenic determinant is a prion protein (PrP) or a dimer thereof, or a prion peptide, and wherein said second attachment site being selected from the group consisting of (i) an attachment site not naturally occurring with said antigen or antigenic determinant; and (ii) an attachment site naturally occurring with said antigen or antigenic determinant; and wherein said second attachment site is capable of association to said first attachment site; and (c) combining said core particle and said at least one antigen or antigenic determinant, wherein said antigen or antigenic determinant and said core particle interact through said association to form an ordered and repetitive antigen array.

In another aspect, the present invention provides for a method of immunization comprising administering the composition of claim 1 or claim 22 to an animal or human.

In a further aspect, the present invention provides for a use of a composition of claim 1 or claim 22 for the manufacture of a medicament for treatment of prion diseases.

In a still further aspect, the present invention provides for a use of a composition of claim 1 or claim 22 for the preparation of a medicament for the therapeutic or prophylactic treatment of prion diseases, preferably of mammalian encephalopathies. Furthermore, in a still further aspect, the present invention provides for a use of a -composition of claim 1 or claim 22, either in isolation or in combination with other agents, for the manufacture of a composition, vaccine, drug or medicament for therapy or prophylaxis of prion diseases, in particular mammalian encephalopathies, and/or for stimulating the mammalian immune system.

Therefore, the invention provides, in particular, vaccine compositions which are suitable for preventing and/or attenuating prion diseases or conditions related thereto. The invention further provides and immunization and vaccination methods, respectively, for preventing and/or attenuating prion diseases or conditions related thereto, in animals, and in particular in cows, sheep and cattles as well as in humans. The inventive compositions may be used prophylactically or therapeutically.

In specific embodiments, the invention provides methods for preventing and/or attenuating prion diseases or conditions related thereto which are caused or exacerbated by “self” gene products, i.e. “self antigens” as used herein. In related embodiments, the invention provides methods for inducing immunological responses in animals and individuals, respectively, which lead to the production of antibodies that prevent and/or attenuate prion diseases or conditions related thereto, which are caused or exacerbated by “self” gene products.

As would be understood by one of ordinary skill in the art, when compositions of the invention are administered to an animal or a human, they may be in a composition which contains salts, buffers, adjuvants, or other substances which are desirable for improving the efficacy of the composition. Examples of materials suitable for use in preparing pharmaceutical compositions are provided in numerous sources including Remington 's Pharmaceutical Sciences (Osol, A, ed., Mack Publishing Co. (1990)).

Compositions of the invention are said to be “pharmacologically acceptable” if their administration can be tolerated by a recipient individual. Further, the compositions of the invention will be administered in a “therapeutically effective amount” (i.e., an amount that produces a desired physiological effect).

The compositions of the present invention may be administered by various methods known in the art, but will normally be administered by injection, infusion, inhalation, oral administration, or other suitable physical methods. The compositions may alternatively be administered intramuscularly, intravenously, or subcutaneously. Components of compositions for administration include sterile aqueous (e.g., physiological saline) or non-aqueous solutions and suspensions. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption.

### BRIEF DESCRIPTION OF THE FIGURES

FIG. 1 shows the cloning and expression of prion protein (cf. Example 13)

FIG. 2 shows the coupling of prion peptides (mPrPl and mPrPs) to Qβ capsid protein; SDS-Page analysis (cf. Example 14).

FIG. 3 shows ELISA of total immunoglobulins (IgG+A+M) recognizing full-length recombinant prion protein detected in serum of mice immunized with mPrPs-Qβ (FIG. 3A) and mPrPl-Qβ (FIG. 3B) (cf. Example 15). Endpoint ELISA titers (log₂-dilution of 20×prediluted serum) were analysed on day 0, day 13 and day 18. Preimmune serum and serum of mice immunized with murine prion peptide cprpshort, murine prion peptide cprplong and Qβ served as negative controls. Serum of transgenic mice expressing anti-PrP antibody (6H4μ) and serum of Prnpo/o mice immunized with mPrPs-Qβ and mPrPl-Qβ were used as positive controls. 50 μg of mPrPs-Qβ and 50 μg of mPrPl-Qβ were administered in alum, as CFA/IFA-emulsion or without adjuvant on day 0, day 14 and day 28. 50 μg of Qβ, 100 μg of cprpshort and 100 μg of cprplong were applied on day 0, day 14 and day 28. Average anti-PrP titers and standard deviation of four mice (mPrPs-Qβ and mPrPl-Qβ) and average anti-PrP titers of two mice (controls) are shown. Immunization with mPrPs-Qβ and mPrPl-Qβ resulted in antibodies recognizing full-length recombinant PrP in wild-type and Prnpo/o mice. The highest anti-PrP serum antibody levels were detected in Prnpo/o mice. Notably, the anti-PrP titer induced by mPrPs-Qβ was higher than the anti-PrP titer induced by mPrPl-Qβ. Moreover, administration of mPrPl-Qβ in alum or emulsified in CFA/IFA seemed to increase the anti-PrP titer. Further, mPrPs-Qβ slightly outreached the titer of 6H4μ-transgenic mice.

FIG. 4 shows long-term anti-PrP antibody response induced by mPrPs-Qβ. Endpoint ELISA titers (log₂-dilution of 20×prediluted serum) of total immunoglobulins (IgG+A+M) recognizing recombinant mouse PrP121-230 are displayed. 50 μg of mPrPs-Qβ, 50 μg of Qβ and 100 μg of murine prion peptide cprpshort were injected subcutaneously on day 0, day 14 and day 28 (indicated by blue arrows). Preimmune serum and serum of mice immunized with murine prion peptide cprpshort and Qβ served as negative controls. Serum of transgenic mice expressing anti-PrP antibody (6H4μ) and serum of Prnpo/o mice immunized with mPrPs-Qβ were used as positive controls. Average anti-PrP titers and standard deviation of four mice (mPrPs-Qβ) and average anti-PrP titers of two mice (controls) are shown.

Three immunizations with mPrPs-Qβ were sufficient to induce an anti-PrP antibody response in wild-type and Prnpo/o mice lasting for two and a half months. Moreover, the anti-PrP serum antibody levels raised by mPrPs-Qβ were slightly higher in Prnpo/o than wild-type mice. Compared to 6H4 μl-transgenic mice, immunization with mPrPs-Qβ resulted in an increased anti-PrP response.

FIG. 5 shows Westernblot-analysis of spleens harvested at 62 dpi. 10% (w/v) homogenates of spleen were prepared and sodium phosphotungstic acid (NaPTA) precipitation was performed. 0.5 mg total protein from spleen homogenates was treated with proteinase K before precipitation. NaPTA-pellet samples were electrophoresed through a 16% SDS-polyacrylamide gel. Proteins were transferred to nitrocellulose and membranes were probed with monoclonal anti-PrP antibody ICSM18. HRP-conjugated rabbit anti-mouse IgG₁ was used as secondary antibody. Bands were detected by enhanced chemiluminescence.

PrP^{Sc} accumulated in scrapie-infected mice that had received no treatment, mPrPs-Qβ or PrPs-peptide. However, the level of deposition of PrP^{Sc} was markedly reduced in mice immunized with mPrPs-Qβ.

FIGS. 6A-C show the purification of AP205 proteins for use in VLPs, as analysed by SDS PAGE and Western-blotting.

FIGS. 7A-C show electron micrographs comparing AP205 phage particles to AP205 virus like particles spontaneously assembled from recombinant protein expressed in E. coli and purified.

### DETAILED DESCRIPTION OF THE INVENTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are hereinafter described.

1. Definitions:

Amino acid linker: An “amino acid linker”, or also just termed “linker” within this specification, as used herein, either associates the antigen or antigenic determinant with the second attachment site, or more preferably, already comprises or contains the second attachment site, typically—but not necessarily—as one amino acid residue, preferably as a cysteine residue. The term “amino acid linker” as used herein, however, does not intend to imply that such an amino acid linker consists exclusively of amino acid residues, even if an amino acid linker consisting of amino acid residues is a preferred embodiment of the present invention. The amino acid residues of the amino acid linker are, preferably, composed of naturally occuring amino acids or unnatural amino acids known in the art, all-L or all-D or mixtures thereof. However, an amino acid linker comprising a molecule with a sulfhydryl group or cysteine residue is also encompassed within the invention. Such a molecule comprise preferably a C1-C6 alkyl-, cycloalkyl (C5,C6), aryl or heteroaryl moiety. However, in addition to an amino acid linker, a linker comprising preferably a C1-C6 alkyl-, cycloalkyl-(C5,C6), aryl- or heteroaryl-moiety and devoid of any amino acid(s) shall also be encompassed within the scope of the invention. Association between the antigen or antigenic determinant or optionally the second attachment site and the amino acid linker is preferably by way of at least one covalent bond, more preferably by way of at least one peptide bond.

Animal: As used herein, the term “animal” is meant to include, for example, humans, sheep, elks, deer, mule deer, minks, mammals, monkeys, horses, cattle, pigs, goats, dogs, cats, rats, mice, birds, chicken, reptiles, fish, insects and arachnids.

Antibody: As used herein, the term “antibody” refers to molecules which are capable of binding an epitope or antigenic determinant. The term is meant to include whole antibodies and antigen-binding fragments thereof, including single-chain antibodies. Most preferably the antibodies are human antigen binding antibody fragments and include, but are not limited to, Fab, Fab′ and F(ab′)2, Fd, single-chain Fvs (scFv), single-chain antibodies, disulfide-linked Fvs (i) and fragments comprising either a V_{L} or V_{H} domain. The antibodies can be from any animal origin including birds and mammals. Preferably, the antibodies are human, murine, rabbit, goat, guinea pig, camel, horse or chicken. As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described, for example, in U.S. Pat. No. 5,939,598 by Kucherlapati et al.

Antigen: As used herein, the term “antigen” refers to a molecule capable of being bound by an antibody or a T cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens.

Antigenic determinant: As used herein, the term “antigenic determinant” is meant to refer to that portion of an antigen that is specifically recognized by either B- or T-lymphocytes. B-lymphocytes responding to antigenic determinants produce antibodies, whereas T-lymphocytes respond to antigenic determinants by proliferation and establishment of effector functions critical for the mediation of cellular and/or humoral immunity.

Association: As used herein, the term “association” as it applies to the first and second attachment sites, refers to the binding of the first and second attachment sites that is preferably by way of at least one non-peptide bond. The nature of the association may be covalent, ionic, hydrophobic, polar or any combination thereof, preferably the nature of the association is covalent.

Attachment Site, First: As used herein, the phrase “first attachment site” refers to an element of non-natural or natural origin, to which the second attachment site located on the antigen or antigenic determinant may associate. The first attachment site may be a protein, a polypeptide, an amino acid, a peptide, a sugar, a polynucleotide, a natural or synthetic polymer, a secondary metabolite or compound (biotin, fluorescein, retinol, digoxigenin, metal ions, phenylmethylsulfonylfluoride), or a combination thereof, or a chemically reactive group thereof. The first attachment site is located, typically and preferably on the surface, of the core particle such as, preferably the virus-like particle. Multiple first attachment sites are present on the surface of the core and virus-like particle, respectively, typically in a repetitive configuration.

Attachment Site, Second: As used herein, the phrase “second attachment site” refers to an element associated with the antigen or antigenic determinant to which the first attachment site located on the surface of the core particle and virus-like particle, respectively, may associate. The second attachment site of the antigen or antigenic determinant may be a protein, a polypeptide, a peptide, a sugar, a polynucleotide, a natural or synthetic polymer, a secondary metabolite or compound (biotin, fluorescein, retinol, digoxigenin, metal ions, phenylmethylsulfonylfluoride), or a combination thereof, or a chemically reactive group thereof. At least one second attachment site is present on the antigen or antigenic determinant. The term “antigen or antigenic determinant with at least one second attachment site” refers, therefore, to an antigen or antigenic construct comprising at least the antigen or antigenic determinant and the second attachment site. However, in particular for a second attachment site, which is of non-natural origin, i.e. not naturally occurring within the antigen or antigenic determinant, these antigen or antigenic constructs comprise an “amino acid linker”.

Bound: As used herein, the term “bound” refers to binding or attachment that may be covalent, e.g., by chemically coupling, or non-covalent, e.g., ionic interactions, hydrophobic interactions, hydrogen bonds, etc. Covalent bonds can be, for example, ester, ether, phosphoester, amide, peptide, imide, carbon-sulfur bonds, carbon-phosphorus bonds, and the like. The term “bound” is broader than and includes terms such as “coupled,” “fused” and “attached”.

Coat protein(s): As used herein, the term “coat protein(s)” refers to the protein(s) of a bacteriophage or a RNA-phage capable of being incorporated within the capsid assembly of the bacteriophage or the RNA-phage. However, when referring to the specific gene product of the coat protein gene of RNA-phages the term “CP” is used. For example, the specific gene product of the coat protein gene of RNA-phage Qβ is referred to as “Qβ CP”, whereas the “coat proteins” of bacteriophage Qβ comprise the “Qβ CP” as well as the A1 protein. The capsid of Bacteriophage Qβ is composed mainly of the Qβ CP, with a minor content of the A1 protein. Likewise, the VLP Qβ coat protein contains mainly Qβ CP, with a minor content of A1 protein.

Core particle: As used herein, the term “core particle” refers to a rigid structure with an inherent repetitive organization. A core particle as used herein may be the product of a synthetic process or the product of a biological process.

Coupled: The term “coupled”, as used herein, refers to attachment by covalent bonds or by strong non-covalent interactions, typically and preferably to attachment by covalent bonds. Any method normally used by those skilled in the art for the coupling of biologically active materials can be used in the present invention.

Effective Amount: As used herein, the term “effective amount” refers to an amount necessary or sufficient to realize a desired biologic effect. An effective amount of the composition would be the amount that achieves this selected result, and such an amount could be determined as a matter of routine by a person skilled in the art. For example, an effective amount for treating an immune system deficiency could be that amount necessary to cause activation of the immune system, resulting in the development of an antigen specific immune response upon exposure to antigen. The term is also synonymous with “sufficient amount.”

The effective amount for any particular application can vary depending on such factors as the disease or condition being treated, the particular composition being administered, the size of the subject, and/or the severity of the disease or condition. One of ordinary skill in the art can empirically determine the effective amount of a particular composition of the present invention without necessitating undue experimentation.

Epitope: As used herein, the term “epitope” refers to continuous or discontinuous portions of a polypeptide having antigenic or immunogenic activity in an animal, preferably a mammal, and most preferably in a human. An epitope is recognized by an antibody or a T cell through its T cell receptor in the context of an MHC molecule. An “immunogenic epitope,” as used herein, is defined as a portion of a polypeptide that elicits an antibody response or induces a T-cell response in an animal, as determined by any method known in the art. (See, for example, Geysen et al., Proc. Natl. Acad. Sci. USA 81:3998-4002 (1983)). The term “antigenic epitope,” as used herein, is defined as a portion of a protein to which an antibody can immunospecifically bind its antigen as determined by any method well known in the art. Immunospecific binding excludes non-specific binding but does not necessarily exclude cross-reactivity with other antigens. Antigenic epitopes need not necessarily be immunogenic. Antigenic epitopes can also be T-cell epitopes, in which case they can be bound immunospecifically by a T-cell receptor within the context of an MHC molecule.

An epitope can comprise 3 amino acids in a spatial conformation which is unique to the epitope. Generally, an epitope consists of at least about 5 such amino acids, and more usually, consists of at least about 8-10 such amino acids. If the epitope is an organic molecule, it may be as small as Nitrophenyl.

Fusion: As used herein, the term “fusion” refers to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini.

Immune response: As used herein, the term “immune response” refers to a humoral immune response and/or cellular immune response leading to the activation or proliferation of B- and/or T-lymphocytes and/or and antigen presenting cells. In some instances, however, the immune responses may be of low intensity and become detectable only when using at least one substance in accordance with the invention. “Immunogenic” refers to an agent used to stimulate the immune system of a living organism, so that one or more functions of the immune system are increased and directed towards the immunogenic agent. An “immunogenic polypeptide” is a polypeptide that elicits a cellular and/or humoral immune response, whether alone or linked to a carrier in the presence or absence of an adjuvant. Preferably, antigen presenting cell may be activated.

A substance which “enhances” an immune response refers to a substance in which an immune response is observed that is greater or intensified or deviated in any way with the addition of the substance when compared to the same immune response measured without the addition of the substance. For example, the lytic activity of cytotoxic T cells can be measured, e.g. using a ⁵¹Cr release assay, in samples obtained with and without the use of the substance during immunization. The amount of the substance at which the CTL lytic activity is enhanced as compared to the CTL lytic activity without the substance is said to be an amount sufficient to enhance the immune response of the animal to the antigen. In a preferred embodiment, the immune response in enhanced by a factor of at least about 2, more preferably by a factor of about 3 or more. The amount or type of cytokines secreted may also be altered. Alternatively, the amount of antibodies induced or their subclasses may be altered.

Immunization: As used herein, the terms “immunize” or “immunization” or related terms refer to conferring the ability to mount a substantial immune response (comprising antibodies and/or cellular immunity such as effector CTL) against a target antigen or epitope. These terms do not require that complete immunity be created, but rather that an immune response be produced which is substantially greater than baseline. For example, a mammal may be considered to be immunized against a target antigen if the cellular and/or humoral immune response to the target antigen occurs following the application of methods of the invention.

Natural origin: As used herein, the term “natural origin” means that the whole or parts thereof are not synthetic and exist or are produced in nature.

Non-natural: As used herein, the term generally means not from nature, more specifically, the term means from the hand of man.

Non-natural origin: As used herein, the term “non-natural origin” generally means synthetic or not from nature; more specifically, the term means from the hand of man.

Ordered and repetitive antigen or antigenic determinant array: As used herein, the term “ordered and repetitive antigen or antigenic determinant array” generally refers to a repeating pattern of antigen or antigenic determinant, characterized by a typically and preferably uniform spacial arrangement of the antigens or antigenic determinants with respect to the core particle and virus-like particle, respectively. In one embodiment of the invention, the repeating pattern may be a geometric pattern. Typical and preferred examples of suitable ordered and repetitive antigen or antigenic determinant arrays are those which possess strictly repetitive paracrystalline orders of antigens or antigenic determinants, preferably with spacings of 1 to 30 nanometers, preferably 5 to 15 nanometers.

Pili: As used herein, the term “pili” (singular being “pilus”) refers to extracellular structures of bacterial cells composed of protein monomers (e.g., pilin monomers) which are organized into ordered and repetitive patterns. Further, pili are structures which are involved in processes such as the attachment of bacterial cells to host cell surface receptors, inter-cellular genetic exchanges, and cell-cell recognition. Examples of pili include Type-1 pili, P-pili, FlC pili, S-pili, and 987P-pili. Additional examples of pili are set out below.

Pilus-like structure: As used herein, the phrase “pilus-like structure” refers to structures having characteristics similar to that of pili and composed of protein monomers. One example of a “pilus-like structure” is a structure formed by a bacterial cell which expresses modified pilin proteins that do not form ordered and repetitive arrays that are identical to those of natural pili.

Polypeptide: As used herein, the term “polypeptide” refers to a molecule composed of monomers (amino acids) linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. This term is also intended to refer to post-expression modifications of the polypeptide, for example, glycosolations, acetylations, phosphorylations, and the like. A recombinant or derived polypeptide is not necessarily translated from a designated nucleic acid sequence. It may also be generated in any manner, including chemical synthesis.

Prion Protein: The term “prion protein (PrP)” as used herein refers to a protein encoded by a PrP gene. Prions are distinct from bacteria, viruses and viroids. Known prions include those which infect animals to cause scrapie, a transmissible, degenerative disease of the nervous system of sheep and goats as well as bovine spongiform encephalopathies (BSE) or mad cow disease and feline spongiform encephalopathies of cats. Five prion diseases known at present to affect humans are (1) kuru, (2) Creutzfeldt-Jakob Disease (CJD), (3) Gerstmann-Straussler-Scheinker Disease (GSS), (4) fatal familial insomnia (FFI) and (5) new variant of CJD (nvCJD). As used herein prion includes all forms of prions causing all or any of these diseases or others in any animals used and in particular in humans, deer, elk and in domesticated farm animals including but not limited to cows and sheep. There are a number of at present known variants to the human PrP gene and the PrP gene of other species. Such variants may be caused by nucleotide point mutations and polymorphisms, respectively, as well as insertions, deletions and/or substitutions of one or more nucleotides, and shall be explicitly encompassed within the scope of the present invention. Therefore, the term “prion protein (PrP)”, as used herein, shall also encompass the prion protein variants, including but not limiting to the above indicated preferred examples.

As used herein, the terms “PrP peptide” or “prion peptide” are broadly defined as any peptide which represents a fraction of a PrP protein and containing at least two, preferably at least three, more prefereably at least four, more prefereably at least five, more prefereably at least six consecutive aminoacids of the original PrP protein. Typically, PrP peptide and “PrP fragment” are used interchangeably. Moreover, the terms “PrP peptide and fragments thereof” or “prion peptide and fragments thereof”, as used herein, shall encompass beside the PrP peptide and prion peptide, respectively, any fraction of said PrP peptide and prion peptide, respectively, wherein said fraction may be, preferably, derived by deletion of one or more amino acids at the N and/or C terminus. The PrP peptide can be obtained by recombinant expression in eukaryotic or prokaryotic expression systems as PrP peptide alone or as a fusion with other amino acids or proteins, e.g. to facilitate folding, expression or solubility of the PrP peptide or to facilitate purification of the PrP peptide. To facilitate or enable correct folding of fusion proteins between PrP peptides and subunit proteins of VLP's or capsids, one or more amino acids may be added N- or C-terminally to PrP petides. To enable coupling of PrP-peptides and subunit proteins of VLP's or capsids, at least one second attachment site may be added to the PrP peptide. Alternatively PrP peptides may be synthesized using methods known to the art. The term-PrP peptide as used herein shall also-encompass a peptide which simulates the three dimensional surface structure of PrP^{C} or PrP_{SC}. Such PrP peptide is not necessarily derived from a continuous amino acid sequence of PrP, but may be formed by discontinuous amino acid residues from PrP. Such peptides may even contain amino acids which are not present in the corresponding PrP protein.

The term “PrP^{C}” as used herein refers to the cellular PrP protein, which is the PrP form that does not typically correlate with the induction of prion disease and which is experimentally defined to be sensitive to Proteinase K digestion. Spectroscopic studies of PrPc showed that PrPc contains ˜40% alpha-helix and is devoid of beta-sheet.

The term “PrP^{SC}” as used herein refers to a post-translationally altered form of PrP^{C}. PrP^{SC} is formed during a process in which PrP^{C} forms a complex with PrP^{SC} and is transformed into a second PrP^{SC}. The amino acid sequence of PrP^{C} before transformation and after transformation to PrP^{SC} is identical. Spectroscopic studies of PrP^{SC} showed that PrP^{SC} contains a high degree of beta-sheet, is at least partially resistant to Proteinase K and correlates with prion disease.

“PrP2” as used herein refers to a mammalian prion-like polypeptide and fragments thereof and allelic variants thereof. PrP2 proteins from human and mouse were identified in part on the basis of homology of the cDNA encoding for PrP2 to PrP of bovine, chicken, human hamster, mouse and sheep as described in WO 01/46419.

Residue: As used herein, the term “residue” is meant to mean a specific amino acid in a polypeptide backbone or side chain.

Self antigen: As used herein, the tem “self antigen” refers to proteins encoded by the host's DNA and products generated by proteins or RNA encoded by the host's DNA are defined as self. In addition, proteins that result from a combination of two or several self-molecules or that represent a fraction of a self-molecule and proteins that have a high homology two self-molecules as defined above (>95%, preferably >97%, more preferably >99%) may also be considered self.

Treatment: As used herein, the terms “treatment”, “treat”, “treated” or “treating” refer to prophylaxis and/or therapy. When used with respect to an infectious disease, for example, the term refers to a prophylactic treatment which increases the resistance of a subject to infection with a pathogen or, in other words, decreases the likelihood that the subject will become infected with the pathogen or will show signs of illness attributable to the infection, as well as a treatment after the subject has become infected in order to fight the infection, e.g., reduce or eliminate the infection or prevent it from becoming worse. When used with respect to prion disease, the term “treatment” refers to a prophylactic or therapeutic treatment which increases the resistance of a subject against, and/or which reverts formation and/or aggregation of the misfolded form of prion protein (Prp^{Sc}) that is associated to prion diseases.

Vaccine: As used herein, the term “vaccine” refers to a formulation which contains the composition of the present invention and which is in a form that is capable of being administered to an animal. Typically, the vaccine comprises a conventional saline or buffered aqueous solution medium in which the composition of the present invention is suspended or dissolved. In this form, the composition of the present invention can be used conveniently to prevent, ameliorate, or otherwise treat a condition. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

Optionally, the vaccine of the present invention additionally includes an adjuvant which can be present in either a minor or major proportion relative to the compound of the present invention. The term “adjuvant” as used herein refers to non-specific stimulators of the immune response or substances that allow generation of a depot in the host which when combined with the vaccine of the present invention provide for an even more enhanced immune response. A variety of adjuvants can be used. Examples include complete and incomplete Freund's adjuvant, aluminum hydroxide and modified muramyldipeptide.

Virus-like particle (VLP): As used herein, the term-“virus-like particle” refers to a structure resembling a virus particle. Moreover, a virus-like particle in accordance with the invention is non replicative and noninfectious since it lacks all or part of the viral genome, in particular the replicative and infectious components of the viral genome. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, or RNA-phage. The terms “viral capsid” or “capsid”, as interchangeably used herein, refer to a macromolecular assembly composed of viral protein subunits. Typically and preferably, the viral protein subunits assemble into a viral capsid and capsid, respectively, having a structure with an inherent repetitive organization, wherein said structure is, typically, spherical or tubular. For example, the capsids of RNA-phages or HBcAg's have a spherical form of icosahedral symmetry. The term “capsid-like structure” as used herein, refers to a macromolecular assembly composed of viral protein subunits ressembling the capsid morphology in the above defined sense but deviating from the typical symmetrical assembly while maintaining a sufficient degree of order and repetitiveness.

Virus-like particle of a bacteriophage: As used herein, the term “virus-like particle of a bacteriophage” refers to a virus-like particle resembling the structure of a bacteriophage, being non replicative and noninfectious, and lacking at least the gene or genes encoding for the replication machinery of the bacteriophage, and typically also lacking the gene or genes encoding the protein or proteins responsible for viral attachment to or entry into the host. This definition should, however, also encompass virus-like particles of bacteriophages, in which the aforementioned gene or genes are still present but inactive, and, therefore, also leading to non-replicative and noninfectious virus-like particles of a bacteriophage.

VLP of RNA phage coat protein: The capsid structure formed from the self-assembly of 180 subunits of RNA phage coat protein and optionally containing host RNA is referred to as a “VLP of RNA phage coat protein”. A specific example is the VLP of Qβ coat protein. In this particular case, the VLP of Qβ coat protein may either be assembled exclusively from Qβ CP subunits (generated by expression of a Qβ CP gene containing, for example, a TAA stop codon precluding any expression of the longer A1 protein through suppression, see Kozlovska, T. M., et al., Intervirology 39: 9-15 (1996)), or additionally contain A1 protein subunits in the capsid assembly.

Virus particle: The term “virus particle” as used herein refers to the morphological form of a virus. In some virus types it comprises a genome surrounded by a protein capsid; others have additional structures (e.g., envelopes, tails, etc.).

One, a, or an: When the terms “one,” “a,” or “an” are used in this disclosure, they mean “at least one” or “one or more,” unless otherwise indicated.

As will be clear to those skilled in the art, certain embodiments of the invention involve the use of recombinant nucleic acid technologies such as cloning, polymerase chain reaction, the purification of DNA and RNA, the expression of recombinant proteins in prokaryotic and eukaryotic cells, etc. Such methodologies are well known to those skilled in the art and can be conveniently found in published laboratory methods manuals (e.g., Sambrook, J. et al., eds., Molecular Cloning, A Laboratory Manual, 2nd. edition, Cold Spring Harbor Laboratory Press, Cold Spring Harbor, N.Y. (1989); Ausubel, F. et al., eds., Current Protocols in Molecular Biology, John H. Wiley & Sons, Inc. (1997)). Fundamental laboratory techniques for working with tissue culture cell lines (Celis, J., ed., Cell Biology, Academic Press, 2^{nd} edition, (1998)) and antibody-based technologies (Harlow, E. and Lane, D., Antibodies: A Laboratory Manual, Cold Spring Harbor Laboratory, Cold Spring Harbor, N.Y. (1988); Deutscher, M. P., “Guide to Protein Purification,” Meth. Enzymol. 128, Academic Press San Diego (1990); Scopes, R. K., Protein Purification Principles and Practice, 3rd ed., Springer-Verlag, New York (1994)) are also adequately described in the literature, all of which are incorporated herein by reference.

2. Compositions and Methods for Enhancing an Immune Response

The disclosed invention provides compositions and methods for enhancing an immune response against prion protein (PrP) or a PrP peptide in an animal. Compositions of the invention comprise, or alternatively consist of (a) a core particle with at least one first attachment site; and (b) at least one antigen or antigenic determinant with at least one second attachment site, wherein said antigen or antigenic determinant is a prion protein (PrP) or a prion peptide, and wherein said second attachment site being selected from the group consisting of (i) an attachment site not naturally occurring with said antigen or antigenic determinant; and (ii) an attachment site naturally occurring with said antigen or antigenic determinant, wherein said second attachment site is capable of association to said first attachment site; and wherein said antigen or antigenic determinant and said core particle interact through said association to form an ordered and repetitive antigen array. More specifically, compositions of the invention comprise, or alternatively consist of, a virus-like particle and at least one antigen or antigenic determinant, wherein the antigen or antigenic determinant is a prion protein (PrP) or a PrP peptide, and wherein the at least one antigen or antigenic determinant is bound to the virus-like particle so as to form an ordered and repetitive antigen-VLP-array. Furthermore, the invention conveniently enables the practitioner to construct such a composition, inter alia, for treatment and/or prophylactic prevention of prion diseases such as iatrogenic CJD, new variant CJD, familial CJD, sporadic CJD, Kuru, GSS, fatal familial insomnia, fatal sporadic insomnia, scrapie, BSE, TME, chronic wasting disease, FSE and exotic ungulate encephalopathy (F. Heppner, A. Aguzzi, Encyclopedia of Life Sciences, 2001, Nature Publishing Group; “Prion Diseases”).

In one embodiment, the core particle-comprises a virus, a bacterial pilus, a structure formed from bacterial pilin, a bacteriophage, a virus-like particle, a viral capsid particle or a recombinant form thereof. Any virus known -in the art having an ordered and repetitive coat and/or core protein structure may be selected as a core particle of the invention; examples of suitable viruses include sindbis and other alphaviruses, rhabdoviruses (e.g. vesicular stomatitis virus), picornaviruses (e.g., human rhino virus, Aichi virus), togaviruses (e.g., rubella virus), orthomyxoviruses (e.g., Thogoto virus, Batken virus, fowl plague virus), polyomaviruses (e.g., polyomavirus BK, polyomavirus JC, avian polyomavirus BFDV), parvoviruses, rotaviruses, Norwalk virus, foot and mouth disease virus, a retrovirus, Hepatitis B virus, Tobacco mosaic virus, Flock House Virus, and human Papilomavirus, and preferably a RNA phage, bacteriophage Qβ, bacteriophage R17, bacteriophage M11, bacteriophage MX1, bacteriophage NL95, bacteriophage fr, bacteriophage GA, bacteriophage SP, bacteriophage MS2, bacteriophage f2, bacteriophage PP7 (for example, see Table 1 in Bachmann, M. F. and Zinkemagel, R. M., Immunol. Today 17:553-558 (1996)).

In a further embodiment, the invention utilizes genetic engineering of a virus to create a fusion between an ordered and repetitive viral envelope protein and a first attachment site comprising a heterologous protein, peptide, antigenic determinant or a reactive amino acid residue of choice. Other genetic manipulations known to those in the art may be included in the construction of the inventive compositions; for example, it may be desirable to restrict the replication ability of the recombinant virus through genetic mutation. Furthermore, the virus used for the present invention is replication incompetent due to chemical or physical inactivation or, as indicated, due to lack of a replication competent genome. The viral protein selected for fusion to the first attachment site should have an organized and repetitive structure. Such an organized and repetitive structure includes paracrystalline organizations with a spacing of 5-30 nm, preferably 5-15 nm, on the surface of the virus. The creation of this type of fusion protein will result in multiple, ordered and repetitive first attachment sites on the surface of the virus and reflect the normal organization of the native viral protein. As will be understood by those in the art, the first attachment site may be or be a part of any suitable protein, polypeptide, sugar, polynucleotide, peptide (amino acid), natural or synthetic polymer, a secondary metabolite or combination thereof that may serve to specifically attach the antigen or antigenic determinant leading an ordered and repetitive antigen array.

In another embodiment of the invention, the core particle is a recombinant alphavirus, and more specifically, a recombinant Sinbis virus. Alphaviruses are positive stranded RNA viruses that replicate their genomic RNA entirely in the cytoplasm of the infected cell and without a DNA intermediate (Strauss, J. and Strauss, E., Microbiol. Rev. 58:491-562 (1994)). Several members of the alphavirus family, Sindbis (Xiong, C. et al., Science 243:1188-1191 (1989); Schlesinger, S., Trends Biotechnol. 11:18-22 (1993)), Semliki Forest Virus (SFV) (Liljeström, P. & Garoff, H., Bio/Technology 9:1356-1361 (1991)) and others (Davis, N. L. et al., Virology 171:189-204 (1989)), have received considerable attention for use as virus-based expression vectors for a variety of different proteins (Lundstrom, K., Curr. Opin. Biotechnol. 8:578-582 (1997); Liljeström, P., Curr. Opin. Biotechnol. 5:495-500 (1994)) and as candidates for vaccine development. Recently, a number of patents have issued directed to the use of alphaviruses for the expression of heterologous proteins and the development of vaccines (see U.S. Pat. Nos. 5,766,602; 5,792,462; 5,739,026; 5,789,245 and 5,814,482). The construction of the alphaviral core particles of the invention may be done by means generally known in the art of recombinant DNA technology, as described by the aforementioned articles, which are incorporated herein by reference.

A variety of different recombinant host cells can be utilized to produce a viral-based core particle for antigen or antigenic determinant attachment. For example, alphaviruses are known to have a wide host range; Sindbis virus infects cultured mammalian, reptilian, and amphibian cells, as well as some insect cells (Clark, H., J. Natl. Cancer Inst. 51:645 (1973); Leake, C., J. Gen. Virol. 35:335 (1977); Stollar, V. in THE TOGAVIRUSES, R. W. Schlesinger, Ed., Academic Press, (1980), pp.583-621). Thus, numerous recombinant host cells can be used in the practice of the invention. BHK, COS, Vero, HeLa and CHO cells are particularly suitable for the production of heterologous proteins because they have the potential to glycosylate heterologous proteins in a manner similar to human cells (Watson, E. et al., Glycobiology 4:227, (1994)) and can be selected (Zang, M. et al., Bio/Technology 13:389 (1995)) or genetically engineered (Renner W. et al., Biotech. Bioeng. 4:476 (1995); Lee K. et al. Biotech. Bioeng. 50:336 (1996)) to grow in serum-free medium, as well as in suspension.

Introduction of the polynucleotide vectors into host cells can be effected by methods described in standard laboratory manuals (see, e.g., Sambrook, J. et al., eds., MOLECULAR CLONING, A LABORATORY MANUAL, 2nd. edition, Cold Spring Harbor Laboratory Press, Cold Spring Harbor, N.Y. (1989), Chapter 9; Ausubel, F. et al., eds., CURRENT PROTOCOLS IN MOLECULAR BIOLOGY, John H. Wiley & Sons, Inc. (1997), Chapter 16), including methods such as electroporation, DEAE-dextran mediated transfection, transfection, microinjection, cationic lipid-mediated transfection, transduction, scrape loading, ballistic introduction, and infection. Methods for the introduction of exogenous DNA sequences into host cells are discussed in Felgner, P. et al., U.S. Pat. No. 5,580,859.

Packaged RNA sequences can also be used to infect host cells. These packaged RNA sequences can be introduced to host cells by adding them to the culture medium. For example, the preparation of non-infective alpahviral particles is described in a number of sources, including “Sindbis Expression System”, Version C (Invitrogen Catalog No. K750-1).

When mammalian cells are used as recombinant host cells for the production of viral-based core particles, these cells will generally be grown in tissue culture. Methods for growing cells in culture are well known in the art (see, e.g., Celis, J., ed., CELL BIOLOGY, Academic Press, 2 edition, (1998); Sambrook, J. et al., eds., MOLECULAR CLONING, A LABORATORY MANUAL, 2nd. edition, Cold Spring Harbor Laboratory Press, Cold Spring Harbor, N.Y. (1989); Ausubel, F. et al., eds., CURRENT PROTOCOLS IN MOLECULAR BIOLOGY, John H. Wiley & Sons, Inc. (1997); Freshney, R., CULTURE OF ANIMAL CELLS, Alan R. Liss, Inc. (1983)).

Further examples of RNA viruses suitable for use as core particle in the present invention include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picomaviridae, including the genus Enterovirus (poliovirus, Coxsackie virus A and B, enteric cytopathic human orphan (ECHO) viruses, hepatitis A, C, D, E and G viruses, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito borne yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuniemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle Disease Virus, Mumps virus), the genus Morbillivirus (Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); the family Rhabdoviridae, including the genus Vesiculovirus (VSV), Chandipura virus, Flanders-Hart Park virus), the genus Lyssavirus (Rabies virus), fish Rhabdoviruses and, filoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that may be used as core particles include, but are not limited to: the family Poxviridae, including the genus Orthopox virus (Variola major, Variola minor, Monkey pox Vaccinia, Cowpox, Buffalopox, Rabbitpox, Ectromelia), the genus Leporipoxvirus (Myxoma, Fibroma), the genus Avipoxvirus (Fowlpox, other avian poxvirus), the genus Capripoxvirus (sheeppox, goatpox), the genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1 and 2, Varicella-Zoster, Equine abortion virus, Equine herpes virus 2 and 3, pseudorabies virus, infectious bovine keratoconjunctivitis virus, infectious bovine rhinotracheitis virus, feline rhinotracheitis virus, infectious laryngotracheitis virus) the Beta-herpesviruses (Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Human subgroups A, B, C, D and E and ungrouped; simian adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovirus, Aleutian mink disease virus, etc.). Finally, DNA viruses may include viruses such as chronic infectious neuropathic agents (CHINA virus).

In other embodiments, a bacterial pilin, a subportion of a bacterial pilin, or a fusion protein which contains either a bacterial pilin or subportion thereof is used to prepare compositions and vaccine compositions, respectively, of the invention. Examples of pilin proteins include pilins produced by Escherichia coli, Haemophilus influenzae, Neisseria meningitidis, Neisseria gonorrhoeae, Caulobacter crescentus, Pseudomonas stutzeri, and Pseudomonas aeruginosa. The amino acid sequences of pilin proteins suitable for use with the present invention include those set out in GenBank reports AJ000636 (SEQ ID NO:1), AJ132364 (SEQ ID NO:2), AF229646 (SEQ ID NO:3), AF051814 (SEQ ID NO:4), AF051815 (SEQ ID NO:5), and X00981 (SEQ ID NO:6), the entire disclosures of which are incorporated herein by reference.

Bacterial pilin proteins are generally processed to remove N-terminal leader sequences prior to export of the proteins into the bacterial periplasm. Further, as one skilled in the art would recognize, bacterial pilin proteins used to prepare compositions and vaccine compositions, respectively, of the invention will generally not have the naturally present leader sequence.

One specific example of a pilin protein suitable for use in the present invention is the P-pilin of E. coli (GenBank report AF237482 (SEQ ID NO:7)). An example of a Type-1 E. coli pilin suitable for use with the invention is a pilin having the amino acid sequence set out in GenBank report P04128 (SEQ ID NO:8), which is encoded by nucleic acid having the nucleotide sequence set out in GenBank report M27603 (SEQ ID NO:9). The entire disclosures of these GenBank reports are incorporated herein by reference. Again, the mature form of the above referenced protein would generally be used to prepare compositions and vaccine compositions, respectively, of the invention.

Bacterial pilins or pilin subportions suitable for use in the practice of the present invention will generally be able to associate to form ordered and repetitive antigen arrays.

Methods for preparing pili and pilus-like structures in vitro are known in the art. Bullitt et al., Proc. Natl. Acad. Sci. USA 93:12890-12895 (1996), for example, describe the in vitro reconstitution of E. coli P-pili subunits. Furthermore, Eshdat et al., J. Bacteriol. 148:308-314 (1981) describe methods suitable for dissociating Type-1 pili of E. coli and the reconstitution of pili. In brief, these methods are as follows: pili are dissociated by incubation at 37° C. in saturated guanidine hydrochloride. Pilin proteins are then purified by chromatography, after which pilin dimers are formed by dialysis against 5 mM tris(hydroxymethyl) aminomethane hydrochloride (pH 8.0). Eshdat et al. also found that pilin dimers reassemble to form pili upon dialysis against the 5 mM tris(hydroxymethyl) aminomethane (pH 8.0) containing 5 mM MgCl₂.

Further, using, for example, conventional genetic engineering and protein modification methods, pilin proteins may be modified to contain a first attachment site to which an antigen or antigenic determinant is linked through a second attachment site. Alternatively, antigens or antigenic determinants can be directly linked through a second attachment site to amino acid residues which are naturally resident in these proteins. These modified pilin proteins may then be used in vaccine compositions of the invention.

Bacterial pilin proteins used to prepare compositions and vaccine compositions, respectively, of the invention may be modified in a manner similar to that described herein for HBcAg. For example, cysteine and lysine residues may be either deleted or substituted with other amino acid residues and first attachment sites may be added to these proteins. Further, pilin proteins may either be expressed in modified form or may be chemically modified after expression. Similarly, intact pili may be harvested from bacteria and then modified chemically.

In another embodiment, pili or pilus-like structures are harvested from bacteria (e.g., E. coli) and used to form compositions and vaccine compositions of the invention. One example of pili suitable for preparing compositions and vaccine compositions is the Type-1 pilus of E. coli, which is formed from pilin monomers having the amino acid sequence set out in SEQ ID NO:8.

A number of methods for harvesting bacterial pili are known in the art. Bullitt and Makowski (Biophys. J. 74:623-632 (1998)), for example, describe a pilus purification method for harvesting P-pili from E. coli. According to this method, pili are sheared from hyperpiliated E. coli containing a P-pilus plasmid and purified by cycles of solubilization and MgCl₂ (1.0 M) precipitation.

Once harvested, pili or pilus-like structures may be modified in a variety of ways. For example, a first attachment site can be added to the pili to which antigens or antigen determinants may be attached through a second attachment site. In other words, bacterial pili or pilus-like structures can be harvested and modified to lead to ordered and repetitive antigen arrays.

Antigens or antigenic determinants could be linked to naturally occurring cysteine resides or lysine residues present in Pili or pilus-like structures. In such instances, the high order and repetitiveness of a naturally occurring amino acid residue would guide the coupling of the antigens or antigenic determinants to the pili or pilus-like structures. For example, the pili or pilus-like structures could be linked to the second attachment sites of the antigens or antigenic determinants using a heterobifunctional cross-linking agent.

When structures which are naturally synthesized by organisms (e.g., pili) are used to prepare compositions and vaccine compositions of the invention, it will often be advantageous to genetically engineer these organisms so that they produce structures having desirable characteristics. For example, when Type-1 pili of E. coli are used, the E. coli from which these pili are harvested may be modified so as to produce structures with specific characteristics. Examples of possible modifications of pilin proteins include the insertion of one or more lysine residues, the deletion or substitution of one or more of the naturally resident lysine residues, and the deletion or substitution of one or more naturally resident cysteine residues (e.g., the cysteine residues at positions 44 and 84 in SEQ ID NO:8).

Further, additional modifications can be made to pilin genes which result in the expression products containing a first attachment site other than a lysine residue (e.g., a FOS or JUN domain). Of course, suitable first attachment sites will generally be limited to those which do not prevent pilin proteins from forming pili or pilus-like structures suitable for use in vaccine compositions of the invention.

Pilin genes which naturally reside in bacterial cells can be modified in vivo (e.g., by homologous recombination) or pilin genes with particular characteristics can be inserted into these cells. For examples, pilin genes could be introduced into bacterial cells as a component of either a replicable cloning vector or a vector which inserts into the bacterial chromosome. The inserted pilin genes may also be linked to expression regulatory control sequences (e.g., a lac operator).

In most instances, the pili or pilus-like structures used in compositions and vaccine compositions, respectively, of the invention will be composed of single type of a pilin subunit. Pili or pilus-like structures composed of identical subunits will generally be used because they are expected to form structures which present highly ordered and repetitive antigen arrays.

However, the compositions of the invention also include compositions and vaccines comprising pili or pilus-like structures formed from heterogenous pilin subunits. The pilin subunits which form these pili or pilus-like structures can be expressed from genes naturally resident in the bacterial cell or may be introduced into the cells. When a naturally resident pilin gene and an introduced gene are both expressed in a cell which forms pili or pilus-like structures, the result will generally be structures formed from a mixture of these pilin proteins. Further, when two or more pilin genes are expressed in a bacterial cell, the relative expression of each pilin gene will typically be the factor which determines the ratio of the different pilin subunits in the pili or pilus-like structures.

When pili or pilus-like structures having a particular composition of mixed pilin subunits is desired, the expression of at least one of the pilin genes can be regulated by a heterologous, inducible promoter. Such promoters, as well as other genetic elements, can be used to regulate the relative amounts of different pilin subunits produced in the bacterial cell and, hence, the composition of the pili or pilus-like structures.

In additional, the antigen or antigenic determinant can be linked to bacterial pili or pilus-like structures by a bond which is not a peptide bond, bacterial cells which produce pili or pilus-like structures used in the compositions of the invention can be genetically engineered to generate pilin proteins which are fused to an antigen or antigenic determinant. Such fusion proteins which form pili or pilus-like structures are suitable for use in vaccine compositions of the invention.

Virus-like particles in the context of the present application refer to structures resembling a virus particle but which are not pathogenic. In general, virus-like particles lack the viral genome and, therefore, are noninfectious. Also, virus-like particles can be produced in large quantities by heterologous expression and can be easily purified.

In a preferred embodiment, the virus-like particle is a recombinant virus-like particle. The skilled artisan can produce VLPs using recombinant DNA technology and virus coding sequences which are readily available to the public. For example, the coding sequence of a virus envelope or core protein can be engineered for expression in a baculovirus expression vector using a commercially available baculovirus vector, under the regulatory control of a virus promoter, with appropriate modifications of the sequence to allow functional linkage of the coding sequence to the regulatory sequence. The coding sequence of a virus envelope or core protein can also be engineered for expression in a bacterial expression vector, for example.

Examples of VLPs include, but are not limited to, the capsid proteins of Hepatitis B virus (Ulrich, et al., Virus Res. 50:141-182 (1998)), measles virus (Warnes, et al., Gene 160:173-178 (1995)), Sindbis virus, rotavirus (U.S. Pat. No. 5,071,651 and U.S. Pat. No. 5,374,426), foot-and-mouth-disease virus (Twomey, et al., Vaccine. 13:1603-1610, (1995)), Norwalk virus (Jiang, X., et al., Science 250:1580-1583 (1990). Matsui, S. M., et al., J. Clin. Invest. 87:1456-1461 (1991)), the retroviral GAG protein (WO 96/30523), the retrotransposon Ty protein p1, the surface protein of Hepatitis B virus (WO 92/11291), human papilloma virus (WO 98/15631), RNA phages, Ty, fr-phage, GA-phage and Qβ-phage.

As will be readily apparent to those skilled in the art, the VLP of the invention is not limited to any specific form. The particle can be synthesized chemically or through a biological process, which can be natural or non-natural. By way of example, this type of embodiment includes a virus-like particle or a recombinant form thereof.

In a more specific embodiment, the VLP can comprise, or alternatively essentially consist of, or alternatively consist of recombinant polypeptides, or fragments thereof, being selected from recombinant polypeptides of Rotavirus, recombinant polypeptides of Norwalk virus, recombinant polypeptides of Alphavirus, recombinant polypeptides of Foot and Mouth Disease virus, recombinant polypeptides of measles virus, recombinant polypeptides of Sindbis virus, recombinant polypeptides of Polyoma virus, recombinant polypeptides of Retrovirus, recombinant polypeptides of Hepatitis B virus (e.g., a HBcAg), recombinant polypeptides of Tobacco mosaic virus, recombinant polypeptides of Flock House Virus, recombinant polypeptides of human Papillomavirus, recombinant polypeptides of bacteriophages, recombinant polypeptides of RNA phages, recombinant polypeptides of Ty, recombinant polypeptides of fr-phage, recombinant polypeptides of GA-phage and recombinant polypeptides of Qβ-phage. The virus-like particle can further comprise, or alternatively essentially consist of, or alternatively consist of, one or more fragments of such polypeptides, as well as variants of such polypeptides. Variants of polypeptides can share, for example, at least 80%, 85%, 90%, 95%, 97%, or 99% identity at the amino acid level with their wild-type counterparts.

In a preferred embodiment, the virus-like particle comprises, consists essentially of, or alternatively consists of recombinant proteins, or fragments thereof, of a RNA-phage. Preferably, the RNA-phage is selected from the group consisting of a) bacteriophage Qβ; b) bacteriophage R17; c) bacteriophage fr; d) bacteriophage GA; e) bacteriophage SP; f) bacteriophage MS2; g) bacteriophage M11; h) bacteriophage MX1; i) bacteriophage NL95; j) bacteriophage f2; and k) bacteriophage PP7.

In another preferred embodiment of the present invention, the virus-like particle comprises, or alternatively consists essentially of, or alternatively consists of recombinant proteins, or fragments thereof, of the RNA-bacteriophage Qβ or of the RNA-bacteriophage fr.

In a further preferred embodiment of the present invention, the recombinant proteins comprise, or alternatively consist essentially of, or alternatively consist of coat proteins of RNA phages.

RNA-phage coat proteins forming capsids or VLP's, or fragments of the bacteriophage coat proteins compatible with self-assembly into a capsid or a VLP, are, therefore, further preferred embodiments of the present invention. Bacteriophage Qβ coat proteins, for example, can be expressed recombinantly in E. coli. Further, upon such expression these proteins spontaneously form capsids. Additionally, these capsids form a structure with an inherent repetitive organization.

Specific preferred examples of bacteriophage coat proteins which can be used to prepare compositions of the invention include the coat proteins of RNA bacteriophages such as bacteriophage Qβ (SEQ ID NO:10; PIR Database, Accession No. VCBPQβ referring to Qβ CP and SEQ ID NO: 11; Accession No. AAA16663 referring to Qβ A1 protein), bacteriophage R17 (SEQ ID NO:12; PIR Accession No. VCBPR7), bacteriophage fr (SEQ ID NO:13; PIR Accession No. VCBPFR), bacteriophage GA (SEQ ID NO:14; GenBank Accession No. NP-040754), bacteriophage SP (SEQ ID NO:15; GenBank Accession No. CAA30374 referring to SP CP and SEQ ID NO: 16; Accession No. referring to SP A1 protein), bacteriophage MS2 (SEQ ID NO:17; PIR Accession No. VCBPM2), bacteriophage M11 (SEQ ID NO:18; GenBank Accession No. AAC06250), bacteriophage MX1 (SEQ ID NO:19; GenBank Accession No. AAC14699), bacteriophage NL95 (SEQ ID NO:20; GenBank Accession No. AAC14704), bacteriophage f2 (SEQ ID NO: 21; GenBank Accession No. P03611), bacteriophage PP7 (SEQ ID NO: 22). Furthermore, the A1 protein of bacteriophage Qβ or C-terminal truncated forms missing as much as 100, 150 or 180 amino acids from its C-terminus may be incorporated in a capsid assembly of Qβ coat proteins. Generally, the percentage of Qβ A1 protein relative to Qβ CP in the capsid assembly will be limited, in order to ensure capsid formation.

Qβ coat protein has also been found to self-assemble into capsids when expressed in E. coli (Kozlovska T M. et al., GENE 137: 133-137 (1993)). The obtained capsids or virus-like particles showed an icosahedral phage-like capsid structure with a diameter of 25 nm and T=3 quasi symmetry. Further, the crystal structure of phage Qβ has been solved. The capsid contains 180 copies of the coat protein, which are linked in covalent pentamers and hexamers by disulfide bridges (Golmohammadi, R. et al., Structure 4: 543-5554 (1996)) leading to a remarkable stability of the capsid of Qβ coat protein. Capsids or VLP's made from recombinant Qβ coat protein may contain, however, subunits not linked via disulfide links to other subunits within the capsid, or incompletely linked. Thus, upon loading recombinant Qβ capsid on non-reducing SDS-PAGE, bands corresponding to monomeric Qβ coat protein as well as bands corresponding to the hexamer or pentamer of Qβ coat protein are visible. Incompletely disulfide-linked subunits could appear as dimer, trimer or even tetramer band in non-reducing SDS-PAGE. Qβ capsid protein also shows unusual resistance to organic solvents and denaturing agents. Surprisingly, we have observed that DMSO and acetonitrile concentrations as high as 30%, and Guanidinium concentrations as high as 1 M do not affect the stability of the capsid. The high stability of the capsid of Qβ coat protein is an advantageous feature, in particular, for its use in immunization and vaccination of mammals and humans in accordance of the present invention.

Upon expression in E. coli, the N-terminal methionine of Qβ coat protein is usually removed, as we observed by N-terminal Edman sequencing as described in Stoll, E., et al., J. Biol. Chem. 252:990-993 (1977). VLP composed from Qβ coat proteins where the N-terminal methionine has not been removed, or VLPs comprising a mixture of Qβ coat proteins where the N-terminal methionine is either cleaved or present are also within the scope of the present invention.

Further RNA phage coat proteins have also been shown to self-assemble upon expression in a bacterial host (Kastelein, RA. et al., Gene 23: 245-254 (1983), Kozlovskaya, T M. et al., Dokl. Akad. Nauk SSSR 287: 452-455 (1986), Adhin, M R. et al., Virology 170: 238-242 (1989), Ni, C Z., et al., Protein Sci. 5: 2485-2493 (1996), Priano, C., et al., J. Mol. Biol. 249: 283-297 (1995)). The Qβ phage capsid contains, in addition to the coat protein, the so called read-through protein A1 and the maturation protein A2. A1 is generated by suppression at the UGA stop codon and has a length of 329 aa. The capsid of phage Qβ recombinant coat protein used in the invention is devoid of the A2 lysis protein, and contains RNA from the host. The coat protein of RNA phages is an RNA binding protein, and interacts with the stem loop of the ribosomal binding site of the replicase gene acting as a translational repressor during the life cycle of the virus. The sequence and structural elements of the interaction are known (Witherell, G W. & Uhlenbeck, O C. Biochemistry 28: 71-76 (1989); Lim F., et al., J. Biol. Chem. 271: 31839-31845 (1996)). The stem loop and RNA in general are known to be involved in the virus assembly (Golmohammadi, R. et al., Structure 4: 543-5554 (1996)).

In a further preferred embodiment of the present invention, the virus-like particle comprises, or alternatively consists essentially of, or alternatively consists of recombinant proteins, or fragments thereof, of a RNA-phage, wherein the recombinant proteins comprise, consist essentially of or alternatively consist of mutant coat proteins of a RNA phage, preferably of mutant coat proteins of the RNA phages mentioned above. In another preferred embodiment, the mutant coat proteins of the RNA phage have been modified by removal of at least one lysine residue by way of substitution, or by addition of at least one lysine residue by way of substitution; alternatively, the mutant coat proteins of the RNA phage have been modified by deletion of at least one lysine residue, or by addition of at least one lysine residue by way of insertion.

In another preferred embodiment, the virus-like particle comprises, or alternatively consists essentially of, or alternatively consists of recombinant proteins, or fragments thereof, of the RNA-bacteriophage Qβ, wherein the recombinant proteins comprise, or alternatively consist essentially of, or alternatively consist of coat proteins having an amino acid sequence of SEQ ID NO:10, or a mixture of coat proteins having amino acid sequences of SEQ ID NO:10 and of SEQ ID NO: 11 or mutants of SEQ ID NO: 11 and wherein the N-terminal methionine is preferably cleaved.

In a further preferred embodiment of the present invention, the virus-like particle comprises, consists essentially of or alternatively consists of recombinant proteins of Qβ, or fragments thereof, wherein the recombinant proteins comprise, or alternatively consist essentially of, or alternatively consist of mutant Qβ coat proteins. In another preferred embodiment, these mutant coat proteins have been modified by removal of at least one lysine residue by way of substitution, or by addition of at least one lysine residue by way of substitution. Alternatively, these mutant coat proteins have been modified by deletion of at least one lysine residue, or by addition of at least one lysine residue by way of insertion.

Four lysine residues are exposed on the surface of the capsid of Qβ coat protein. Qβ mutants, for which exposed lysine residues are replaced by arginines can also be used for the present invention. The following Qβ coat protein mutants and mutant Qβ VLP's can, thus, be used in the practice of the invention: “Qβ-240” (Lys13-Arg; SEQ ID NO:23), “Qβ-243” (Asn 10-Lys; SEQ ID NO:24), “Qβ-250” (Lys 2-Arg, Lys13-Arg; SEQ ID NO:25), “Qβ-251” (SEQ ID NO:26) and “Qβ-259” (Lys 2-Arg, Lys16-Arg; SEQ ID NO:27). Thus, in further preferred embodiment of the present invention, the virus-like particle comprises, consists essentially of or alternatively consists of recombinant proteins of mutant Qβ coat proteins, which comprise proteins having an amino acid sequence selected from the group of a) the amino acid sequence of SEQ ID NO:23; b) the amino acid sequence of SEQ ID NO:24; c) the amino acid sequence of SEQ ID NO:25; d) the amino acid sequence of SEQ ID NO:26; and e) the amino acid sequence of SEQ ID NO:27. The construction, expression and purification of the above indicated Qβ coat proteins, mutant Qβ coat protein VLP's and capsids, respectively, are disclosed in pending U.S. application Ser. No. 10/050,902 filed by the present assignee on Jan. 18, 2002. In particular is hereby referred to Example 18 of above mentioned application.

In a further preferred embodiment of the present invention, the virus-like particle comprises, or alternatively consists essentially of, or alternatively consists of recombinant proteins of Qβ, or fragments thereof, wherein the recombinant proteins comprise, consist essentially of or alternatively consist of a mixture of either one of the foregoing Qβ mutants and the corresponding A1 protein.

In a further preferred embodiment of the present invention, the virus-like particle comprises, or alternatively essentially consists of, or alternatively consists of recombinant proteins, or fragments thereof, of RNA-phage AP205.

The AP205 genome consists of a maturation protein, a coat protein, a replicase and two open reading frames not present in related phages; a lysis gene and an open reading frame playing a role in the translation of the maturation gene (Klovins, J., et al., J. Gen. Virol. 83: 1523-33 (2002)). AP205 coat protein can be expressed from plasmid pAP283-58 (SEQ ID NO: 94), which is a derivative of pQb10 (Kozlovska, T. M. et al., Gene 137:133-37 (1993)), and which contains an AP205 ribosomal binding site. Alternatively, AP205 coat protein may be cloned into pQb185, downstream of the ribosomal binding site present in the vector. Both approaches lead to expression of the protein and formation of capsids as described in the co-pending US provisional patent application with the title “Molecular Antigen Arrays” and having filed by the present assignee on Jul. 16, 2002, which is incorporated by reference in its entirety. Vectors pQb10 and pQb185 are vectors derived from pGEM vector, and expression of the cloned genes in these vectors is controlled by the trp promoter (Kozlovska, T. M. et al., Gene 137:133-37 (1993)). Plasmid pAP283-58 (SEQ ID NO:94) comprises a putative AP205 ribosomal binding site in the following sequence, which is downstream of the XbaI site, and immediately upstream of the ATG start codon of the AP205 coat protein: tctagaATTTTCTGCGCACCCAT CCCGGGTGGCGCCCAAAGTGAGGAAAATCACatg (bases 77-133 of SEQ ID NO: 94). The vector pQb185 comprises a Shine Delagarno sequence downstream from the XbaI site and upstream of the start codon (tctagaTTAACCCAACGCGTAGGAGTCAGGCCatg, SEQ ID NO: 98, Shine Delagarno sequence underlined).

In a further preferred embodiment of the present invention, the virus-like particle comprises, or alternatively essentially consists of, or alternatively consists of recombinant coat proteins, or fragments thereof, of the RNA-phage AP205.

This preferred embodiment of the present invention, thus, comprises AP205 coat proteins that form capsids. Such proteins are recombinantly expressed, or prepared from natural sources. AP205 coat proteins produced in bacteria spontaneously form capsids, as evidenced by Electron Microscopy (EM) and immunodiffusion. The structural properties of the capsid formed by the AP205 coat protein (SEQ ID NO: 95) and those formed by the coat protein of the AP205 RNA phage are nearly indistinguishable when seen in EM. AP205 VLPs are highly immunogenic, and can be linked with antigens and/or antigenic determinants to generate vaccine constructs displaying the antigens and/or antigenic determinants oriented in a repetitive manner. High titers are elicited against the so displayed antigens showing that bound antigens and/or antigenic determinants are accessible for interacting with antibody molecules and are immunogenic.

In a further preferred embodiment of the present invention, the virus-like particle comprises, or alternatively essentially consists of, or alternatively consists of recombinant mutant coat proteins, or fragments thereof, of the RNA-phage AP205.

Assembly-competent mutant forms of AP205 VLPs, including AP205 coat protein with the subsitution of proline at amino acid 5 to threonine (SEQ ID NO: 96), may also be used in the practice of the invention and leads to a further preferred embodiment of the invention. These VLPs, AP205 VLPs derived from natural sources, or AP205 viral particles, may be bound to antigens to produce ordered repetitive arrays of the antigens in accordance with the present invention.

AP205 P5-T mutant coat protein can be expressed from plasmid pAP281-32 (SEQ ID No. 97), which is derived directly from pQb185, and which contains the mutant AP205 coat protein gene instead of the Qβ coat protein gene. Vectors for expression of the AP205 coat protein are transfected into E. coli for expression of the AP205 coat protein.

Methods for expression of the coat protein and the mutant coat protein, respectively, leading to self-assembly into VLPs are described in co-pending US provisional patent application with the title “Molecular Antigen Arrays” and having filed by the present assignee on Jul. 16, 2002, which is incorporated by reference in its entirety. Suitable E. coli strains include, but are not limited to, E. coli K802, JM 109, RR1. Suitable vectors and strains and combinations thereof can be identified by testing expression of the coat protein and mutant coat protein, respectively, by SDS-PAGE and capsid formation and assembly by optionally first purifying the capsids by gel filtration and subsequently testing them in an immunodiffusion assay (Ouchterlony test) or Electron Microscopy (Kozlovska, T. M., el al., Gene 137:133-37 (1993)).

AP205 coat proteins expressed from the vectors pAP283-58 and pAP281-32 may be devoid of the initial Methionine amino-acid, due to processing in the cytoplasm of E. coli. Cleaved, uncleaved forms of AP205 VLP, or mixtures thereof are further preferred embodiments of the invention.

In a further preferred embodiment of the present invention, the virus-like particle comprises, or alternatively essentially consists of, or alternatively consists of a mixture of recombinant coat proteins, or fragments thereof, of the RNA-phage AP205 and of recombinant mutant coat proteins, or fragments thereof, of the RNA-phage AP205.

In a further preferred embodiment of the present invention, the virus-like particle comprises, or alternatively essentially consists of, or alternatively consists of fragments of recombinant coat proteins or recombinant mutant coat proteins of the RNA-phage AP205.

Recombinant AP205 coat protein fragments capable of assembling into a VLP and a capsid, respectively are also useful in the practice of the invention. These fragments may be generated by deletion, either internally or at the termini of the coat protein and mutant coat protein, respectively. Insertions in the coat protein and mutant coat protein sequence or fusions of antigen sequences to the coat protein and mutant coat protein sequence, and compatible with assembly into a VLP, are further embodiments of the invention and lead to chimeric AP205 coat proteins, and particles, respectively. The outcome of insertions, deletions and fusions to the coat protein sequence and whether it is compatible with assembly into a VLP can be determined by electron microscopy.

The particles formed by the AP205 coat protein, coat protein fragments and chimeric coat proteins described above, can be isolated in pure form by a combination of fractionation steps by precipitation and of purification steps by gel filtration using e.g. Sepharose CL-4B, Sepharose CL-2B, Sepharose CL-6B columns and combinations thereof as described in the co-pending US provisional patent application with the title “Molecular Antigen Arrays” and having filed by the present assignee on Jul. 16, 2002, which is incorporated by reference in its entirety. Other methods of isolating virus-like particles are known in the art, and may be used to isolate the virus-like particles (VLPs) of bacteriophage AP205. For example, the use of ultracentrifugation to isolate VLPs of the yeast retrotransposon Ty is described in U.S. Pat. No. 4,918,166, which is incorporated by reference herein in its entirety.

The crystal structure of several RNA bacteriophages has been determined (Golmohammadi, R. et al., Structure 4:543-554 (1996)). Using such information, surface exposed residues can be identified and, thus, RNA-phage coat proteins can be modified such that one or more reactive amino acid residues can be inserted by way of insertion or substitution. As a consequence, those modified forms of bacteriophage coat proteins can also be used for the present invention. Thus, variants of proteins which form capsids or capsid-like structures (e.g., coat proteins of bacteriophage Qβ, bacteriophage R17, bacteriophage fr, bacteriophage GA, bacteriophage SP, and bacteriophage MS2) can also be used to prepare compositions of the present invention.

Although the sequence of the variants proteins discussed above will differ from their wild-type counterparts, these variant proteins will generally retain the ability to form capsids or capsid-like structures. Thus, the invention further includes compositions and vaccine compositions, respectively, which further includes variants of proteins which form capsids or capsid-like structures, as well as methods for preparing such compositions and vaccine compositions, respectively, individual protein subunits used to prepare such compositions, and nucleic acid molecules which encode these protein subunits. Thus, included within the scope of the invention are variant forms of wild-type proteins which form capsids or capsid-like structures and retain the ability to associate and form capsids or capsid-like structures.

As a result, the invention further includes compositions and vaccine compositions, respectively, comprising proteins, which comprise, or alternatively consist essentially of, or alternatively consist of amino acid sequences which are at least 80%, 85%, 90%, 95%, 97%, or 99% identical to wild-type proteins which form ordered arrays and having an inherent repetitive structure, respectively.

Further included within the scope of the invention are nucleic acid molecules which encode proteins used to prepare compositions of the present invention.

In other embodiments, the invention further includes compositions comprising proteins, which comprise, or alternatively consist essentially of, or alternatively consist of amino acid sequences which are at least 80%, 85%, 90%, 95%, 97%, or 99% identical to any of the amino acid sequences shown in SEQ ID NOs:10-27.

Proteins suitable for use in the present invention also include C-terminal truncation mutants of proteins which form capsids or capsid-like structures, or VLP's. Specific examples of such truncation mutants include proteins having an amino acid sequence shown in any of SEQ ID NOs:10-27 where 1, 2, 5, 7, 9, 10, 12, 14, 15, or 17 amino acids have been removed from the C-terminus. Typically, theses C-terminal truncation mutants will retain the ability to form capsids or capsid-like structures.

Further proteins suitable for use in the present invention also include N-terminal truncation mutants of proteins which form capsids or capsid-like structures. Specific examples of such truncation mutants include proteins having an amino acid sequence shown in any of SEQ ID NOs:10-27 where 1, 2, 5, 7, 9, 10, 12, 14, 15, or 17 amino acids have been removed from the N-terminus. Typically, these N-terminal truncation mutants will retain the ability to form capsids or capsid-like structures.

Additional proteins suitable for use in the present invention include N- and C-terminal truncation mutants which form capsids or capsid-like structures. Suitable truncation mutants include proteins having an amino acid sequence shown in any of SEQ ID NOs:10-27 where 1, 2, 5, 7, 9, 10, 12, 14, 15, or 17 amino acids have been removed from the N-terminus and 1, 2, 5, 7, 9, 10, 12, 14, 15, or 17 amino acids have been removed from the C-terminus. Typically, these N-terminal and C-terminal truncation mutants will retain the ability to form capsids or capsid-like structures.

The invention further includes compositions comprising proteins which comprise, or alternatively consist essentially of, or alternatively consist of, amino acid sequences which are at least 80%, 85%, 90%, 95%, 97%, or 99% identical to the above described truncation mutants.

The invention thus includes compositions and vaccine compositions prepared from proteins which form capsids or VLP's, methods for preparing these compositions from individual protein subunits and VLP's or capsids, methods for preparing these individual protein subunits, nucleic acid molecules which encode these subunits, and methods for vaccinating and/or eliciting immunological responses in individuals using these compositions of the present invention.

As previously stated, the invention includes virus-like particles or recombinant forms thereof. In one further preferred embodiment, the particles used in compositions of the invention are composed of a Hepatitis B core protein (HBcAg) or a fragment of a HBcAg. In a further embodiment, the particles used in compositions of the invention are composed of a Hepatitis B core protein (HBcAg) or a fragment of a HBcAg protein, which has been modified to either eliminate or reduce the number of free cysteine residues. Zhou et al. (J. Virol. 66:5393-5398 (1992)) demonstrated that HBcAgs which have been modified to remove the naturally resident cysteine residues retain the ability to associate and form capsids. Thus, VLP's suitable for use in compositions of the invention include those comprising modified HBcAgs, or fragments thereof, in which one or more of the naturally resident cysteine residues have been either deleted or substituted with another amino acid residue (e.g., a serine residue).

The HBcAg is a protein generated by the processing of a Hepatitis B core antigen precursor protein. A number of isotypes of the HBcAg have been identified and their amino acids sequences are readily available to those skilled in the art. In most instances, compositions and vaccine compositions, respectively, of the invention will be prepared using the processed form of a HBcAg (i.e., a HBcAg from which the N-terminal leader sequence of the Hepatitis B core antigen precursor protein have been removed).

Further, when HBcAgs are produced under conditions where processing will not occur, the HBcAgs will generally be expressed in “processed” form. For example, when an E. coli expression system directing expression of the protein to the cytoplasm is used to produce HBcAgs of the invention, these proteins will generally be expressed such that the N-terminal leader sequence of the Hepatitis B core antigen precursor protein is not present.

The preparation of Hepatitis B virus-like particles, which can be used for the present invention, is disclosed, for example, in WO 00/32227, and hereby in particular in Examples 17 to 19 and 21 to 24, as well as in WO 01/85208, and hereby in particular in Examples 17 to 19, 21 to 24, 31 and 41, and in pending U.S. application Ser. No. 10/050,902 filed by the present assignee on Jan. 18, 2002. For the latter application, it is in particular referred to Example 23, 24, 31 and 51. All three documents are explicitly incorporated herein by reference.

The present invention also includes HBcAg variants which have been modified to delete or substitute one or more additional cysteine residues. It is known in the art that free cysteine residues can be involved in a number of chemical side reactions. These side reactions include disulfide exchanges, reaction with chemical substances or metabolites that are, for example, injected or formed in a combination therapy with other substances, or direct oxidation and reaction with nucleotides upon exposure to UV light. Toxic adducts could thus be generated, especially considering the fact that HBcAgs have a strong tendency to bind nucleic acids. The toxic adducts would thus be distributed between a multiplicity of species, which individually may each be present at low concentration, but reach toxic levels when together.

In view of the above, one advantage to the use of HBcAgs in vaccine compositions which have been modified to remove naturally resident cysteine residues is that sites to which toxic species can bind when antigens or antigenic determinants are attached would be reduced in number or eliminated altogether.

A number of naturally occurring HBcAg variants suitable for use in the practice of the present invention have been identified. Yuan et al., (J. Virol. 73:10122-10128 (1999)), for example, describe variants in which the isoleucine residue at position corresponding to position 97 in SEQ ID NO:28 is replaced with either a leucine residue or a phenylalanine residue. The amino acid sequences of a number of HBcAg variants, as well as several Hepatitis B core antigen precursor variants, are disclosed in GenBank reports AAF121240 (SEQ ID NO:29), AF121239 (SEQ ID NO:30), X85297 (SEQ ID NO:31), X02496 (SEQ ID NO:32), X85305 (SEQ ID NO:33), X85303 (SEQ ID NO:34), AF151735 (SEQ ID NO:35), X85259 (SEQ ID NO:36), X85286 (SEQ ID NO:37), X85260 (SEQ ID NO:38), X85317 (SEQ ID NO:39), X85298 (SEQ ID NO:40), AF043593 (SEQ ID NO:41), M20706 (SEQ ID NO:42), X85295 (SEQ ID NO:43), X80925 (SEQ ID NO:44), X85284 (SEQ ID NO:45), X85275 (SEQ ID NO:46), X72702 (SEQ ID NO:47), X85291 (SEQ ID NO:48), X65258 (SEQ ID NO:49), X85302 (SEQ ID NO:50), M32138 (SEQ ID NO:51), X85293 (SEQ ID NO:52), X85315 (SEQ ID NO:53), U95551 (SEQ ID NO:54), X85256 (SEQ ID NO:55), X85316 (SEQ ID NO:56), X85296 (SEQ ID NO:57), AB033559 (SEQ ID NO:58), X59795 (SEQ ID NO:59), X85299 (SEQ ID NO:60), X85307 (SEQ ID NO:61), X65257 (SEQ ID NO:62), X85311 (SEQ ID NO:63), X85301 (SEQ ID NO:64), X85314 (SEQ ID NO:65), X85287 (SEQ ID NO:66), X85272 (SEQ ID NO:67), X85319 (SEQ ID NO:68), AB010289 (SEQ ID NO:69), X85285 (SEQ ID NO:70), AB010289 (SEQ ID NO:71), AF121242 (SEQ ID NO:72), M90520 (SEQ ID NO:73), P03153 (SEQ ID NO:74), AF110999 (SEQ ID NO:75), and M95589 (SEQ ID NO:76), the disclosures of each of which are incorporated herein by reference. These HBcAg variants differ in amino acid sequence at a number of positions, including amino acid residues which corresponds to the amino acid residues located at positions 12, 13, 21, 22, 24, 29, 32, 33, 35, 38, 40, 42, 44, 45, 49, 51, 57, 58, 59, 64, 66, 67, 69, 74, 77, 80, 81, 87, 92, 93, 97, 98, 100, 103, 105, 106, 109, 113, 116, 121, 126, 130, 133, 135, 141, 147, 149, 157, 176, 178, 182 and 183 in SEQ ID NO:77. Further HBcAg variants suitable for use in the compositions of the invention, and which may be further modified according to the disclosure of this specification are described in WO 00/198333, WO 00/177158 and WO 00/214478.

As noted above, generally processed HBcAgs (i.e., those which lack leader sequences) will be used in the compositions and vaccine compositions, respectively, of the invention. The present invention includes vaccine compositions, as well as methods for using these compositions, which employ the above described variant HBcAgs.

Whether the amino acid sequence of a polypeptide has an amino acid sequence that is at least 80%, 85%, 90%, 95%, 97% or 99% identical to one of the above wild-type amino acid sequences, or a subportion thereof, can be determined conventionally using known computer programs such the Bestfit program. When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed.

The HBcAg variants and precursors having the amino acid sequences set out in SEQ ID NOs: 29-72 and 73-76 are relatively similar to each other. Thus, reference to an amino acid residue of a HBcAg variant located at a position which corresponds to a particular position in SEQ ID NO:77, refers to the amino acid residue which is present at that position in the amino acid sequence shown in SEQ ID NO:77. The homology between these HBcAg variants is for the most part high enough among Hepatitis B viruses that infect mammals so that one skilled in the art would have little difficulty reviewing both the amino acid sequence shown in SEQ ID NO:77 and that of a particular HBcAg variant and identifying “corresponding” amino acid residues. Furthermore, the HBcAg amino acid sequence shown in SEQ ID NO:73, which shows the amino acid sequence of a HBcAg derived from a virus which infect woodchucks, has enough homology to the HBcAg having the amino acid sequence shown in SEQ ID NO:77 that it is readily apparent that a three amino acid residue insert is present in SEQ ID NO:64 between amino acid residues 155 and 156 of SEQ ID NO:77.

The invention also includes vaccine compositions which comprise HBcAg variants of Hepatitis B viruses which infect birds, as wells as vaccine compositions which comprise fragments of these HBcAg variants. For these HBcAg variants one, two, three or more of the cysteine residues naturally present in these polypeptides could be either substituted with another amino acid residue or deleted prior to their inclusion in vaccine compositions of the invention.

As discussed above, the elimination of free cysteine residues reduces the number of sites where toxic components can bind to the HBcAg, and also eliminates sites where cross-linking of lysine and cysteine residues of the same or of neighboring HBcAg molecules can occur. Therefore, in another embodiment of the present invention, one or more cysteine residues of the Hepatitis B virus capsid protein have been either deleted or substituted with another amino acid residue.

In other embodiments, compositions and vaccine compositions, respectively, of the invention will contain HBcAgs from which the C-terminal region (e.g., amino acid residues 145-185 or 150-185 of SEQ ID NO:77) has been removed. Thus, additional modified HBcAgs suitable for use in the practice of the present invention include C-terminal truncation mutants. Suitable truncation mutants include HBcAgs where 1, 5, 10, 15, 20, 25, 30, 34, 35, amino acids have been removed from the C-terminus.

HBcAgs suitable for use in the practice of the present invention also include N-terminal truncation mutants. Suitable truncation mutants include modified HBcAgs where 1, 2, 5, 7, 9, 10, 12, 14, 15, or 17 amino acids have been removed from the N-terminus.

Further HBcAgs suitable for use in the practice of the present invention include N- and C-terminal truncation mutants. Suitable truncation mutants include HBcAgs where 1, 2, 5, 7, 9, 10, 12, 14, 15, and 17 amino acids have been removed from the N-terminus and 1, 5, 10, 15, 20, 25, 30, 34, 35 amino acids have been removed from the C-terminus.

The invention further includes compositions and vaccine compositions, respectively, comprising HBcAg polypeptides comprising, or alternatively essentially consisting of, or alternatively consisting of, amino acid sequences which are at least 80%, 85%, 90%, 95%, 97%, or 99% identical to the above described truncation mutants.

In certain embodiments of the invention, a lysine residue is introduced into a HBcAg polypeptide, to mediate the binding of the prion protein, prion protein domain or PrP peptide to the VLP of HBcAg. In preferred embodiments, compositions of the invention are prepared using a HBcAg comprising, or alternatively consisting of, amino acids 1-144, or 1-149, 1-185 of SEQ ID NO:77, which is modified so that the amino acids corresponding to positions 79 and 80 are replaced with a peptide having the amino acid sequence of Gly-Gly-Lys-Gly-Gly (SEQ ID NO:78). In further preferred embodiments, the cysteine residues at positions 48 and 107 of SEQ ID NO:77 are mutated to serine. The invention further includes compositions comprising the corresponding polypeptides having amino acid sequences shown in any of SEQ ID NOs:29-74, which also have above noted amino acid alterations. Further included within the scope of the invention are additional HBcAg variants which are capable of associating to form a capsid or VLP and have the above noted amino acid alterations. Thus, the invention further includes compositions and vaccine compositions, respectively, comprising HBcAg polypeptides which comprise, or alternatively consist of, amino acid sequences which are at least 80%, 85%, 90%, 95%, 97% or 99% identical to any of the wild-type amino acid sequences, and forms of these proteins which have been processed, where appropriate, to remove the N-terminal leader sequence and modified with above noted alterations.

Compositions or vaccine compositions of the invention may comprise mixtures of different HBcAgs. Thus, these vaccine compositions may be composed of HBcAgs which differ in amino acid sequence. For example, vaccine compositions could be prepared comprising a “wild-type” HBcAg and a modified HBcAg in which one or more amino acid residues have been altered (e.g., deleted, inserted or substituted). Further, preferred vaccine compositions of the invention are those which present highly ordered and repetitive antigen array, wherein the antigen is a prion protein, prion protein domain or prion peptide.

In a further preferred embodiment of the present invention, the at least one prion protein (PrP), PrP peptide or PrP domain is bound to said core particle and virus-like particle, respectively, by at least one covalent bond. Preferably, the least one prion protein (PrP), PrP peptide or PrP domain is bound to the core particle and virus-like particle, respectively, by at least one covalent bond, said covalent bond being a non-peptide bond leading to a core particle-PrP ordered and repetitive array and a PrP-VLP-array or -conjugate, respectively. This PrP-VLP array and conjugate, respectively, has typically and preferably a repetitive and ordered structure since the at least one, but usually more than one, prion protein (PrP), PrP peptide or PrP domain is bound to the VLP in an oriented manner. Preferably, more than 10, 20, 40, 80, 120 PrP peptides or proteins are bound to the VLP. The formation of a repetitive and ordered PrP-VLP array and conjugate, respectively, is ensured by an oriented and directed as well as defined binding and attachment, respectively, of the at least one prion protein (PrP), PrP peptide or PrP domain to the VLP as will become apparent in the following. Furthermore, the typical inherent highly repetitive and organized structure of the VLP's advantageously contributes to the display of the prion protein (PrP), PrP peptide or PrP domain in a highly ordered and repetitive fashion leading to a highly organized and repetitive PrP-VLP array and conjugate, respectively.

Therefore, the preferred inventive conjugates and arrays, respectively, differ from prior art conjugates in their highly organized structure, dimensions, and in the repetitiveness of the antigen on the surface of the array. The preferred embodiment of this invention, furthermore, allows expression of both the particle and the antigen in an expression host guaranteeing proper folding of the antigen, i.e. the at least one prion protein (PrP), PrP peptide or PrP domain, and proper folding and assembly of the VLP.

The present invention discloses methods of binding of prion protein, prion protein domain or prion peptide to core particles and VLPs, repectively. As indicated, in one aspect of the invention, the prion protein, prion protein domain or prion peptide is bound to the core particle and VLP, respectively, by way of chemical cross-linking, typically and preferably by using a heterobifunctional cross-linker. Several hetero-bifunctional cross-linkers are known to the art. In preferred embodiments, the hetero-bifunctional cross-linker contains a functional group which can react with preferred first attachment sites, i.e. with the side-chain amino group of lysine residues of the core particle and the VLP or at least one VLP subunit, respectively, and a further functional group which can react with a preferred second attachment site, i.e. a cysteine residue naturally present, made available for reaction by reduction, or engineered on the prion protein, prion protein domain or prion peptide, and optionally also made available for reaction by reduction. The first step of the procedure, typically called the derivatization, is the reaction of the core particle or the VLP with the cross-linker. The product of this reaction is an activated core particle or activated VLP, also called activated carrier. In the second step, unreacted cross-linker is removed using usual methods such as gel filtration or dialysis. In the third step, the prion protein, prion protein domain or prion peptide is reacted with the activated carrier, and this step is typically called the coupling step. Unreacted prion protein, prion protein domain or prion peptide may be optionally removed in a fourth step, for example by dialysis. Several hetero-bifunctional cross-linkers are known to the art. These include the preferred cross-linkers SMPH (Pierce), Sulfo-MBS, Sulfo-EMCS, Sulfo-GMBS, Sulfo-SIAB, Sulfo-SMPB, Sulfo-SMCC, SVSB, SIA and other cross-linkers available for example from the Pierce Chemical Company (Rockford, Ill., USA), and having one functional group reactive towards amino groups and one functional group reactive towards cysteine residues. The above mentioned cross-linkers all lead to formation of a thioether linkage. Another class of cross-linkers suitable in the practice of the invention is characterized by the introduction of a disulfide linkage between the prion protein, prion protein domain or prion peptide and the core particle or VLP upon coupling. Preferred cross-linkers belonging to this class include for example SPDP and Sulfo-LC-SPDP (Pierce). The extent of derivatization of the core particle and VLP, respectively, with cross-linker can be influenced by varying experimental conditions such as the concentration of each of the reaction partners, the excess of one reagent over the other, the pH, the temperature and the ionic strength. The degree of coupling, i.e. the amount of prion protein, prion protein domain or prion peptides per subunits of the core particle and VLP, respectively, can be adjusted by varying the experimental conditions described above to match the requirements of the vaccine. Solubility of the prion protein, prion protein domain or prion peptide may impose a limitation on the amount of prion protein, prion protein domain or prion peptide that can be coupled on each subunit, and in those cases where the obtained vaccine would be insoluble, reducing the amount of prion protein, prion protein domain or prion peptides per subunit is beneficial.

A particularly favored method of binding of prion protein, prion protein domain or prion peptides to the core particle and the VLP, respectively, is the linking of a lysine residue on the surface of the core particle and the VLP, respectively, with a cysteine residue on the prion protein, prion protein domain or prion peptide. Thus, in a preferred embodiment of the present invention, the first attachment site is a lysine residue and the second attachment site is a cysteine residue. In some embodiments, engineering of an amino acid linker containing a cysteine residue, as a second attachment site or as a part thereof, to the prion protein, prion protein domain or prion peptide for coupling to the core particle and VLP, respectively, may be required. Alternatively, a cysteine may be introduced either by insertion or mutation within the prion protein, prion protein domain or prion peptide. Alternatively, the cysteine residue may be introduced by chemical coupling.

The selection of the amino acid linker will be dependent on the nature of the antigen and self-antigen, respectively, i.e. on the nature of the PrP protein, PrP peptide or PrP domain, on its biochemical properties, such as pI, charge distribution and glycosylation. In general, flexible amino acid linkers are favored. Preferred embodiments of the amino acid linker are selected from the group consisting of: (a) CGG; (b) N-terminal gamma 1-linker; (c) N-terminal gamma 3-linker; (d) Ig hinge regions; (e) N-terminal glycine linkers; (f) (G)ₖC(G)ₙ with n=0-12 and k=0-5; (g) N-terminal glycine-serine linkers; (h) (G)ₖC(G)ₘ(S)₁(GGGGS)ₙ with n=0-3, k=0-5, m=0-10, 1=0-2 (SEQ ID NO: 99); (i) GGC; (k) GGC-NH2; (1) C-terminal gamma 1-linker; (m) C-terminal gamma 3-linker; (n) C-terminal glycine linkers; (o) (G)ₙC(G)ₖ with n=0-12 and k=0-5; (p) C-terminal glycine-serine linkers; (q) (G)ₘ(S)₁(GGGGS)ₙ(G)ₒC(G)ₖ with n=0-3, k=0-5, m=0-10, 1=0-2, and o=0-8 (SEQ ID NO:100).

Further preferred examples of amino acid linkers are the hinge region of Immunoglobulins, glycine serine linkers (GGGGS)ₙ (SEQ ID NO:101), and glycine linkers (G)ₙ all further containing a cysteine residue as second attachment site and optionally further glycine residues. Typically preferred examples of said amino acid linkers are N-terminal gamma 1: CGDKTHTSPP (SEQ ID NO:102); C-terminal gamma 1: DKTHTSPPCG (SEQ ID NO:103); N-terminal gamma 3: CGGPKPSTPPGSSGGAP (SEQ ID NO:104); C-terminal gamma 3: PKPSTPPGSSGGAPGGCG (SEQ ID NO:105); N-terminal glycine linker: GCGGGG (SEQ ID NO:106); C-terminal glycine linker: GGGGCG (SEQ ID NO:107); C-terminal glycine-lysine linker: GGKKGC (SEQ ID NO:108); N-terminal glycine-lysine linker: CGKKGG (SEQ ID NO:109).

In a further preferred embodiment of the present invention, and in particular if the antigen is a prion peptide, GGCG (SEQ ID NO: 110), GGC or GGC-NH2 (“NH2” stands for amidation) linkers at the C-terminus of the peptide or CGG at its N-terminus are preferred as amino acid linkers. In general, glycine residues will .be inserted between bulky amino acids and the cysteine to be used as second attachment site, to avoid potential steric hindrance of the bulkier amino acid in the coupling reaction.

The cysteine residue present on the prion protein, prion protein domain or prion peptide has to be in its reduced state to react with the hetero-bifunctional cross-linker on the activated VLP, that is a free cysteine or a cysteine residue with a free sulfhydryl group has to be available. In the instance where the cysteine residue to function as binding site is in an oxidized form, for example if it is forming a disulfide bridge, reduction of this disulfide bridge with e.g. DTT, TCEP or β-mercaptoethanol is required.

Binding of the prion protein, prion protein domain or prion peptide to the core particle and VLP, respectively, by using a hetero-bifunctional cross-linker according to the preferred methods described above, allows coupling of the prion protein, prion protein domain or prion peptide to the core particle and the VLP, respectively, in an oriented fashion. Other methods of binding the prion protein, prion protein domain or prion peptide to the core particle and the VLP, respectively, include methods wherein the prion protein, prion protein domain or prion peptide is cross-linked to the core particle and the VLP, respectively, using the carbodiimide EDC, and NHS. The prion protein, prion protein domain or prion peptide may also be first thiolated through reaction, for example with SATA, SATP or iminothiolane. The prion protein, prion protein domain or prion peptide, after deprotection if required, may then be coupled to the core particle and the VLP, respectively, as follows. After separation of the excess thiolation reagent, the prion protein, prion protein domain or prion peptide is reacted with the core particle and the VLP, respectively, previously activated with a hetero-bifunctional cross-linker comprising a cysteine reactive moiety, and therefore displaying at least one or several functional groups reactive towards cysteine residues, to which the thiolated prion protein, prion protein domain or prion peptide can react, such as described above. Optionally, low amounts of a reducing agent are included in the reaction mixture. In further methods, the prion protein, prion protein domain or prion peptide is attached to the core particle and the VLP, respectively, using a homo-bifunctional cross-linker such as glutaraldehyde, DSG, BM[PEO]₄, BS³, (Pierce Chemical Company, Rockford, Ill., USA) or other known homo-bifunctional cross-linkers whith functional groups reactive towards amine groups or carboxyl groups of the core particle and the VLP, respectively.

In a further embodiment, the prion protein, or prion protein domain is bound to the core particle and the VLP, respectively, through modification of the carbohydrate moieties present on glycosylated prion protein or prion domain and subsequent reaction with the core particle and the VLP, respectively. In one embodiment, the glycosylated prion protein or prion protein domain is reacted with sodium periodate in a mild oxidation reaction of the carbohydrate moiety, to yield an activated prion protein or prion protein domain with one or more aldehyde functional groups. The so activated prion protein or prion protein domain is separated from excess sodium periodate, and further reacted with the core particle and the VLP, respectively, wherein lysine residues of the core particle and the VLP, respectively, or of at least one VLP subunit are reacting with the previously formed aldehyde functional group on the prion protein or prion protein domain, for example as described by Hermanson, G. T. in

Bioconjugate Techniques, Academic Press Inc., San. Diego, Calif., USA. Self polymerization of the activated prion protein or prion protein domain may be controlled by adjusting the pH as described in the aforementioned publication. The formed Schiff base is preferably further reduced with sodium cyanoborohydride, which is subsequently removed by gel filtration or dialysis. Alternatively, the core particle and the VLP, respectively, may be reacted with EDC at carboxyl groups of the core particle and the VLP, respectively, or at least one VLP subunit and a dihydrazyde, such as adipic acid dihydrazyde, to yield a hydrazide moiety available for reaction with the one or more aldehyde functional groups present on the activated prion protein or prion protein domain. The so formed hydrazone may be further reduced with sodium cyanoborohydride. Alternatively, the activated prion protein or prion protein domain with one or more aldehyde functional groups is reacted with cysteamine, resulting in-the introduction of a cysteine group in the prion protein or prion protein domain. Additional cross-linking methods and cross-linkers, suitable for binding a prion protein, prion protein domain or prion peptide to a core particle and a VLP, respectively, as well as guidance on performing the coupling reactions and on the use of chemical cross-linkers and chemical cross-linking procedures can be found in Hermanson, G. T. in Bioconjugate Techniques, Academic Press Inc., San Diego, Calif., USA.

Other methods of binding the VLP to a prion protein, prion protein domain or prion peptide include methods where the core particle and the VLP, respectively, is biotinylated, and the prion protein, prion protein domain or prion peptide expressed as a streptavidin-fusion protein, or methods wherein both the prion protein, prion protein domain or prion peptides and the core particle and the VLP, respectively, are biotinylated, for example as described in WO 00/23955. In this case, the prion protein, prion protein domain or prion peptide may be first bound to streptavidin or avidin by adjusting the ratio of prion protein, prion protein domain or prion peptide to streptavidin such that free binding sites are still available for binding of the core particle and the VLP, respectively, which is added in the next step. Alternatively, all components may be mixed in a “one pot” reaction. Other ligand-receptor pairs, where a soluble form of the receptor and of the ligand is available, and are capable of being cross-linked to the core particle and the VLP, respectively, or the prion protein, prion protein domain or prion peptide, may be used as binding agents for binding the prion protein, prion protein domain or prion peptide to the core particle and the VLP, respectively. Alternatively, either the ligand or the receptor may be fused to the prion protein, prion protein domain or prion peptide, and so mediate binding to the core particle and the VLP, respectively, chemically bound or fused either to the receptor, or the ligand respectively. Fusion may also be effected by insertion or substitution.

As already indicated, in a favored embodiment of the present invention, the VLP is the VLP of a RNA phage, and in a more preferred embodiment, the VLP is the VLP of RNA phage Qβ coat protein.

One or several antigen molecules, i.e. a prion peptide, a prion protein or a prion protein domain, can be attached to one subunit of the capsid or VLP of RNA phages coat proteins, preferably through the exposed lysine residues of the VLP of RNA phages, if sterically allowable. A specific feature of the VLP of the coat protein of RNA phages and in particular of the Qβ coat protein VLP is thus the possibility to couple several antigens per subunit. This allows for the generation of a dense antigen array.

In a preferred embodiment of the invention, the binding and attachment, respectively, of the at least prion protein (PrP) or PrP peptide or PrP domain to the core particle and the virus-like particle, respectively, is by way of interaction and association, respectively, between at least one first attachment site of the virus-like particle and at least one second attachment of the antigen or antigenic determinant.

VLPs or capsids of Qβ coat protein display a defined number of lysine residues on their surface, with a defined topology with three lysine residues pointing towards the interior of the capsid and interacting with the RNA, and four other lysine residues exposed to the exterior of the capsid. These defined properties favor the attachment of antigens to the exterior of the particle, rather than to the interior of the particle where the lysine residues interact with RNA. VLPs of other RNA phage coat proteins also have a defined number of lysine residues on their surface and a defined topology of these lysine residues.

In further preferred embodiments of the present invention, the first attachment site is a lysine residue and/or the second attachment comprises sulfhydryl group or a cysteine residue. In a very preferred embodiment of the present invention, the first attachment site is a lysine residue and the second attachment is a cysteine residue.

In very preferred embodiments of the invention, the -prion protein, prion protein domain or prion peptide is bound via a cysteine residue, either naturally present on the prion protein, prion protein domain or prion peptide or engineered, to lysine residues of the VLP of RNA phage coat protein, and in particular to the VLP of Qβ coat protein.

Another advantage of the VLPs derived from RNA phages is their high expression yield in bacteria that allows production of large quantities of material at affordable cost.

As indicated, the inventive conjugates and arrays, respectively, differ from prior art conjugates in their highly organized structure, dimensions, and in the repetitiveness of the antigen on the surface of the array. Moreover, the use of the VLPs as carriers allow the formation of robust antigen arrays and conjugates, respectively, with variable antigen density. In particular, the use of VLP's of RNA phages, and hereby in particular the use of the VLP of RNA phage Qβ coat protein allows to achieve very high epitope density. The preparation of compositions of VLPs of RNA phage coat proteins with a high epitope density can be effected by using the teaching of this application.

The second attachment site, as defined herein, may be either naturally or non-naturally present with the antigen or the antigenic determinant. In the case of the absence of a suitable natural occurring second attachment site on the antigen or antigenic determinant, such a, then non-natural second attachment has to be engineered to the antigen.

As described above, four lysine residues are exposed on the surface of the VLP of Qβ coat protein. Typically these residues are derivatized upon reaction with a cross-linker molecule. In the instance where not all of the exposed lysine residues can be coupled to an antigen, the lysine residues which have reacted with the cross-linker are left with a cross-linker molecule attached to the ε-amino group after the derivatization step. This leads to disappearance of one or several positive charges, which may be detrimental to the solubility and stability of the VLP. By replacing some of the lysine residues with arginines, as in the disclosed Q coat protein mutants described below, we prevent the excessive disappearance of positive charges since the arginine residues do not react with the cross-linker. Moreover, replacement of lysine residues by arginines may lead to more defined antigen arrays, as fewer sites are available for reaction to the antigen.

Accordingly, exposed lysine residues were replaced by arginines in the following Qβ coat protein mutants and mutant Qβ VLPs disclosed in this application: Qβ-240 (Lys13-Arg; SEQ ID NO:23), Qβ-250 (Lys 2-Arg, Lys13-Arg; SEQ ID NO:25) and Qβ-259 (Lys 2-Arg, Lys16-Arg; SEQ ID NO:27). The constructs were cloned, the proteins expressed, the VLPs purified and used for coupling to peptide and protein antigens. Qβ-251; (SEQ ID NO:26) was also constructed, and guidance on how to express, purify and couple the VLP of Qβ-251 coat protein can be found throughout the application.

In a further embodiment, we disclose a Qβ mutant coat protein with one additional lysine residue, suitable for obtaining even higher density arrays of antigens. This mutant Qβ coat protein, Qβ-243 (Asn 10-Lys; SEQ ID NO:24), was cloned, the protein expressed, and the capsid or VLP isolated and purified, showing that introduction of the additional lysine residue is compatible with self-assembly of the subunits to a capsid or VLP. Thus, prion protein, prion protein domain or prion petide arrays and conjugates, respectively, may be prepared using VLP of Qβ coat protein mutants. A particularly favored method of attachment of antigens to VLPs, and in particular to VLPs of RNA phage coat proteins is the linking of a lysine residue present on the surface of the VLP of RNA phage coat proteins with a cysteine residue naturally present or engineered on the antigen, i.e. the prion protein or prion peptide. In order for a cysteine residue to be effective as second attachment site, a sulfhydryl group must be available for coupling. Thus, a cysteine residue has to be in its reduced state, that is, a free cysteine or a cysteine residue with a free sulfhydryl group has to be available. In the instant where the cysteine residue to function as second attachment site is in an oxidized form, for example if it is forming a disulfide bridge, reduction of this disulfide bridge with e.g. DTT, TCEP or mercaptoethanol is required. The concentration of reductand, and the molar excess of reductand over antigen has to be adjusted for each antigen. A titration range, starting from concentrations as low as 10 μM or lower, up to 10 to 20 mM or higher reductand if required is tested, and coupling of the antigen to the carrier assessed. Although low concentrations of reductand are compatible with the coupling reaction as described in pending U.S. application Ser. No. 10/050,902 filed by the present assignee on Jan. 18, 2002, higher concentrations inhibit the coupling reaction, as a skilled artisan would know, in which case the reductand has to be removed by dialysis or gel filtration. Advantageously, the pH of the dialysis or equilibration buffer is lower than 7, preferably 6. The compatibility of the low pH buffer with antigen activity or stability has to be tested.

Epitope density on the VLP of RNA phage coat proteins can be modulated by the choice of cross-linker and other reaction conditions. For example, the cross-linkers Sulfo-GMBS and SMPH typically allow reaching high epitope density. Derivatization is positively influenced by high concentration of reactands, and manipulation of the reaction conditions can be used to control the number of antigens coupled to VLPs of RNA phage coat proteins, and in particular to VLPs of Qβ coat protein.

Prior to the design of a non-natural second attachment site the position at which it should be fused, inserted or generally engineered has to be chosen. The selection of the position of the second attachment site may, by way of example, be based on a crystal structure of the antigen. Such a crystal structure of the antigen may provide information on the availability of the C- or N-termini of the molecule (determined for example from their accessibility to solvent), or on the exposure to solvent of residues suitable for use as second attachment sites, such as cysteine residues. Exposed disulfide bridges, as is the case for Fab fragments, may also be a source of a second attachment site, since they can be generally converted to single cysteine residues through mild reduction. Mild reduction conditions not affecting the immunogenicity of prion peptide or prion protein will be choosen. In general, in the case where immunization with a self-antigen is aiming at inhibiting the interaction of this self-antigen with its natural ligands, the second attachment site will be added such that it allows generation of antibodies against the site of interaction with the natural ligands. Thus, the location of the second attachment site will be selected such that steric hindrance from the second attachment site or any amino acid linker containing the same is avoided. In further embodiments, an antibody response directed at a site distinct from the interaction site of the self-antigen with its natural ligand is desired. In such embodiments, the second attachment site may be selected such that it prevents generation of antibodies against the interaction site of the self-antigen with its natural ligands.

Other criteria in selecting the position of the second attachment site include the oligomerization state of the antigen, the site of oligomerization, the presence of a cofactor, and the availability of experimental evidence disclosing sites in the antigen structure and sequence where modification of the antigen is compatible with the function of the self-antigen, or with the generation of antibodies recognizing the self-antigen.

In the most preferred embodiments, the prion protein or prion peptide comprises a single second attachment site or a single reactive attachment site capable of association with the first attachment sites on the core particle and the VLPs or VLP subunits, respectively. This ensures a defined and uniform binding and association, respectively, of the at least one, but typically more than one, preferably more than 10, 20, 40, 80, 120 antigens to the core particle and VLP, respectively. The provision of a single second attachment site or a single reactive attachment site on the antigen, thus, ensures a single and uniform type of binding and association, respectively leading to a very highly ordered and repetitive array. For example, if the binding and association, respectively, is effected by way of a lysine—(as the first attachment site) and cysteine—(as a second attachment site) interaction, it is ensured, in accordance with this preferred embodiment of the invention, that only one cysteine residue per antigen, independent whether this cysteine residue is naturally or non-naturally present on the antigen, is capable of binding and associating, respectively, with the VLP and the first attachment site of the core particle, respectively.

In some embodiments, engineering of a second attachment site onto the antigen require the fusion of an amino acid linker containing an amino acid suitable as second attachment site according to the disclosures of this invention. Therefore, in a preferred embodiment of the present invention, an amino acid linker is bound to the antigen or the antigenic determinant by way of at least one covalent bond. Preferably, the amino acid linker comprises, or alternatively consists of, the second attachment site. In a further preferred embodiment, the amino acid linker comprises a sulfhydryl group or a cysteine residue. In another preferred embodiment, the amino acid linker is cysteine. Some criteria of selection of the amino acid linker as well as further preferred embodiments of the amino acid linker according to the invention have already mentioned above.

In a further preferred embodiment of the invention, the at least one antigen or antigenic determinant, i.e. the PrP protein, PrP peptide or the PrP domain is fused to the core particle and the virus-like particle, respectively. As outlined above, a VLP is typically composed of at least one subunit assembling into a VLP. Thus, in again a further preferred embodiment of the invention, the antigen or antigenic determinant, preferably the at least one prion peptide or PrP domain, is fused to at least one subunit of the virus-like particle or of a protein capable of being incorporated into a VLP generating a chimeric VLP-subunit-prion peptide protein fusion.

Fusion of the prion peptides can be effected by insertion into the VLP subunit sequence, or by fusion to either the N- or C-terminus of the VLP-subunit or protein capable of being incorporated into a VLP. Hereinafter, when referring to fusion proteins of a peptide to a VLP subunit, the fusion to either ends of the subunit sequence or internal insertion of the peptide within the subunit sequence are encompassed.

Fusion may also be effected by inserting the prion peptide sequences into a variant of a VLP subunit where part of the subunit sequence has been deleted, that are further referred to as truncation mutants. Truncation mutants may have N- or C-terminal, or internal deletions of part of the sequence of the VLP subunit. For example, the specific VLP HBcAg with, for example, deletion of amino acid residues 79 to 81 is a truncation mutant with an internal deletion. Fusion of prion peptides to either the N- or C-terminus of the truncation mutants VLP-subunits also lead to embodiments of the invention. Likewise, fusion of an epitope into the sequence of the VLP subunit may also be effected by substitution, where for example for the specific VLP HBcAg, amino acids 79-81 are replaced with a foreign epitope. Thus, fusion, as referred to hereinafter, may be effected by insertion of the prion peptide sequence in the sequence of a VLP subunit, by substitution of part of the sequence of the VLP subunit with the prion peptide sequence, or by a combination of deletion, substitution or insertions.

The chimeric prion peptide-VLP subunit will be in general capable of self-assembly into a VLP. VLP displaying epitopes fused to their subunits are also herein referred to as chimeric VLPs. As indicated, the virus-like particle comprises or alternatively is composed of at least one VLP subunit. In a further embodiment of the invention, the virus-like particle comprises or alternatively is composed of a mixture of chimeric VLP subunits and non-chimeric VLP subunits, i.e. VLP subunits not having an antigen fused thereto, leading to so called mosaic particles. This may be advantageous to ensure formation of and assembly to a VLP. In those embodiments, the proportion of chimeric VLP-subunits may be 1, 2, 5, 10, 20, 30, 40, 50, 60, 70, 80, 90, 95% or higher.

Flanking amino acid residues may be added to either end of the sequence of the peptide or epitope to be fused to either end of the sequence of the subunit of a VLP, or for internal insertion of such peptidic sequence into the sequence of the subunit of a VLP. Glycine and serine residues are particularly favored amino acids to be used in the flanking sequences added to the prion peptide to be fused. Glycine residues confer additional flexibility, which may diminish the potentially destabilizing effect of fusing a foreign sequence into the the sequence of a VLP subunit.

In a specific embodiment of the invention, the VLP is a Hepatitis B. core antigen VLP. Fusion proteins to either the N-terminus of a HBcAg (Neyrinck, S. et al., Nature Med. 5:1157-1163 (1999)) or insertions in the so called major immunodominant region (MIR) have been described (Pumpens, P. and Grens, E., Intervirology 44:98-114 (2001)), WO 01/98333), and are preferred embodiments of the invention. Naturally occurring variants of HBcAg with deletions in the. MIR have also been described (Pumpens, P. and Grens, E., Intervirology 44:98-114 (2001), which is expressly incorporated by reference in their entirety), and fusions to the N- or C-terminus, as well as insertions at the position of the MIR corresponding to the site of deletion as compared to a wt HBcAg are further embodiments of the invention. Fusions to the C-terminus have also been described (Pumpens, P. and Grens, E., Intervirology 44:98-114 (2001)). One skilled in the art will easily find guidance on how to construct fusion proteins using classical molecular biology techniques (Sambrook, J. et al., eds., Molecular Cloning, A Laboratory Manual, 2nd. edition, Cold Spring Habor Laboratory Press, Cold Spring Harbor, N.Y. (1989), Ho et al., Gene 77:51 (1989)). Vectors and plasmids encoding HBcAg and HBcAg fusion proteins and useful for the expression of a HBcAg and HBcAg fusion proteins have been described (Pumpens, P. & Grens, E. Intervirology 44: 98-114 (2001), Neyrinck, S. et al., Nature Med. 5:1157-1163 (1999)) and can be used in the practice of the invention. We also describe by way of example (Example 6) the insertion of an epitope into the MIR of HBcAg, resulting in a chimeric self-assembling HBcAg. An important factor for the optimization of the efficiency of self-assembly and of the display of the epitope to be-inserted in the MIR of HBcAg is the choice of the insertion site, as well as the number of amino acids to be deleted from the HBcAg sequence within the MIR (Pumpens, P. and Grens, E., Intervirology 44:98-114 (2001); EP 421,635; U.S. Pat. No. 6,231,864) upon insertion, or in other words, which amino acids form HBcAg are to be substituted with the new epitope. For example, substitution of HBcAg amino acids 76-80, 79-81, 79-80, 75-85 or 80-81 with foreign epitopes has been described (Pumpens, P. and Grens, E., Intervirology 44:98-114 (2001); EP0421635; U.S. Pat. No. 6,231,864). HBcAg contains a long arginine tail (Pumpens, P. and Grens, E., Intervirology 44:98-114 (2001)) which is dispensable for capsid assembly and capable of binding nucleic acids (Pumpens, P. and Grens, E., Intervirology 44:98-114 (2001)). HBcAg either comprising or lacking this arginine tail are both embodiments of the invention.

In a further preferred embodiment of the invention, the VLP is a VLP of a RNA phage. The major coat proteins of RNA phages spontaneously assemble into VLPs upon expression in bacteria, and in particular in E. coli. Specific examples of bacteriophage coat proteins which can be used to prepare compositions of the invention include the coat proteins of RNA bacteriophages such as bacteriophage Qβ (SEQ ID NO:10; PIR Database, Accession No. VCBPQβ referring to Qβ CP and SEQ ID NO: 11; Accession No. AAA16663 referring to Qβ A1 protein) and bacteriophage fr (SEQ ID NO:13; PIR Accession No. VCBPFR).

In a more preferred embodiment, the at least one prion peptide is fused to a Qβ coat protein. Fusion protein constructs wherein epitopes have been fused to the C-terminus of a truncated form of the A1 protein of Qβ, or inserted within the A1 protein have been described (Kozlovska, T. M., et al., Intervirology, 39:9-15 (1996)). The A1 protein is generated by suppression at the UGA stop codon and has a length of 329 aa, or 328 aa, if the cleavage of the N-terminal methionine is taken into account. Cleavage of the N-terminal methionine before an alanine (the second amino acid encoded by the Qβ CP gene) usually takes place in E. coli, and such is the case for N-termini of the Qβ coat proteins CP. The part of the A1 gene, 3′ of the UGA amber codon encodes the CP extension, which has a length of 195 amino acids. Insertion of the at least one prion peptide between position 72 and 73 of the CP extension leads to further embodiments of the invention (Kozlovska, T. M., et al., Intervirology 39:9-15 (1996)). Fusion of a prion peptide at the C-terminus of a C-terminally truncated Qβ A1 protein leads to further preferred embodiments of the invention. For example, Kozlovska et al., (Intervirology, 39: 9-15 (1996)) describe Qβ A1 protein fusions where the epitope is fused at the C-terminus of the Qβ CP extension truncated at position 19.

As described by Kozlovska et al. (Intervirology 39: 9-15 (1996)), assembly of the particles displaying the fused epitopes typically requires the presence of both the A1 protein-prion peptide fusion and the wt CP to form a mosaic particle. However, embodiments comprising virus-like particles, and hereby in particular the VLPs of the RNA phage Qβ coat protein, which are exclusively composed of VLP subunits having at least one prion peptide fused thereto, are also within the scope of the present invention.

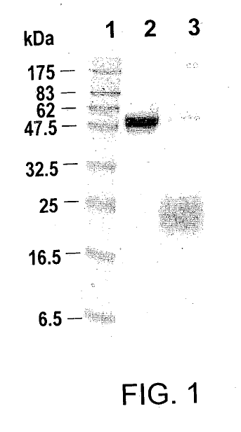

The production of mosaic particles may be effected in a number of ways. Kozlovska et al., Intervirolog, 39:9-15 (1996), describe two methods, which both can be used in the practice of the invention. In the first approach, efficient display of the fused epitope on the VLPs is mediated by the expression of the plasmid encoding the Qβ A1 protein fusion having a UGA stop codong between CP and CP extension in a E. coli strain harboring a plasmid encoding a cloned UGA suppressor tRNA which leads to translation of the UGA codon into Trp (pISM3001 plasmid (Smiley B. K., et al., Gene 134:33-40 (1993))). In another approach, the CP gene stop codon is modified into UAA, and a second plasmid expressing the A1 protein-prion peptide fusion is cotransformed. The second plasmid encodes a different antibiotic resistance and the origin of replication is compatible with the first plasmid (Kozlovska, T. M., et al., Intervirology 39:9-15 (1996)). In a third approach, CP and the A1 protein-prion peptide fusion are encoded in a bicistronic manner, operatively linked to a promoter such as the Trp promoter, as described in FIG. 1 of Kozlovska et al., Intervirology, 39:9-15 (1996).

In a further embodiment, the prion peptide is inserted between amino acid 2 and 3 (numbering of the cleaved CP, that is wherein the N-terminal methionine is cleaved) of the fr CP, thus leading to a prion peptide-fr CP fusion protein. Vectors and expression systems for construction and expression of fr CP fusion proteins self-assembling to VLP and useful in the practice of the invention have been described (Pushko P. et al., Prot. Eng. 6:883-891 (1993)). In a specific embodiment, the prion peptide sequence is inserted into a deletion variant of the fr CP after amino acid 2, wherein residues 3 and 4 of the fr CP have been deleted (Pushko P. et al., Prot. Eng. 6:883-891 (1993)).

Fusion of epitopes in the N-terminal protuberant β-hairpin of the coat protein of RNA phage MS-2 and subsequent presentation of the fused epitope on the self-assembled VLP of RNA phage MS-2 has also been described (WO 92/13081), and fusion of prion peptide by insertion or substitution into the coat protein of MS-2 RNA phage is also falling under the scope of the invention.

In another embodiment of the invention, the prion peptides are fused to a capsid protein of papillomavirus. In a more specific embodiment, the prion peptides are fused to the major capsid protein L1 of bovine papillomavirus type 1 (BPV-1). Vectors and expression systems for construction and expression of BPV-1 fusion proteins in a baculovirus/insect cells systems have been described (Chackerian, B. et al., Proc. Natl. Acad. Sci. USA 96:2373-2378 (1999), WO 00/23955). Substitution of amino acids 130-136 of BPV-1 L1 with a prion peptide leads to a BPV-1 L1-prion peptide fusion protein, which is a preferred embodiment of the invention. Cloning in a baculovirus vector and expression in baculovirus infected Sf9 cells has been described, and can be used in the practice of the invention (Chackerian, B. et al., Proc. Natl. Acad. Sci. USA 96:2373-2378 (1999), WO 00/23955). Purification of the assembled particles displaying the fused prion peptides can be performed in a number of ways, such as for example gel filtration or sucrose gradient ultracentrifugation (Chackerian, B. et al., Proc. Natl. Acad. Sci. USA 96:2373-2378 (1999), WO 00/23955).

In a further embodiment of the invention, the prion peptides are fused to a Ty protein capable of being incorporated into a Ty VLP. In a more specific embodiment, the prion peptides are fused to the p1 or capsid protein encoded by the TYA gene (Roth, J. F., Yeast 16:785-795 (2000)). The yeast retrotransposons Ty1, 2, 3 and 4 have been isolated from Saccharomyces Serevisiae, while the retrotransposon Tf1 has been isolated from Schizosaccharomyces Pombae (Boeke, J. D. and Sandmeyer, S. B., “Yeast Transposable elements,” in The molecular and Cellular Biology of the Yeast Saccharomyces: Genome dynamics, Protein Synthesis, and Energetics., p. 193, Cold Spring Harbor Laboratory Press (1991)). The retrotransposons Ty1 and 2 are related to the copia class of plant and animal elements, while Ty3 belongs to the gypsy family of retrotransposons, which is related to plants and animal retroviruses. In the Ty1 retrotransposon, the p1 protein, also referred to as Gag or capsid protein, has a length of 440 amino acids. P1 is cleaved during maturation of the VLP at position 408, leading to the p2 protein, the essential component of the VLP.

Fusion proteins to p1 and vectors for the expression of said fusion proteins in Yeast have been described (Adams, S. E., et al., Nature 329:68-70 (1987)). So, for example, a prion protein peptide may be fused to p1 by inserting a sequence coding for the prion protein peptide into the BamH1 site of the pMA5620 plasmid (Adams, S. E., et al., Nature 329:68-70 (1987)). The cloning of sequences coding for foreign epitopes into the pMA5620 vector leads to expression of fusion proteins comprising amino acids 1-381 of p1 of Ty1-15, fused C-terminally to the N-terminus of the foreign epitope. Likewise, N-terminal fusion of prion peptides, or internal insertion into the p1 sequence, or substitution of part of the p1 sequence is also meant to fall within the scope of the invention. In particular, insertion of prion peptides into the Ty sequence between amino acids 30-31, 67-68, 113-114 and 132-133 of the Ty protein p1 (EP0677111) leads to preferred embodiments of the invention.

Further VLPs suitable for fusion of prion peptides are, for example, Retrovirus-like-particles (WO9630523), HIV2 Gag (Kang, Y. C., et al, Biol. Chem. 380:353-364 (1999)), Cowpea Mosaic Virus (Taylor, K. M. et al., Biol. Chem. 380:387-392 (1999)), parvovirus VP2 VLP (Rueda, P. et al., Virology 263:89-99 (1999)), HBsAg (U.S. Pat. No. 4,722,840, EP0020416B 1).

Examples of chimeric VLPs suitable for the practice of the invention are also those described in Intervirology 39:1 (1996). Further examples of VLPs contemplated for use in the invention are: HPV-1, HPV-6, HPV-11, HPV-16, PV-18, HPV-33, HPV-45, CRPV, COPV, HIV GAG, Tobacco Mosaic Virus. Virus-like particles of SV-40, Polyomavirus, Adenovirus, Herpes Simplex Virus, Rotavirus and -Norwalk virus have -also been made, and chimeric VLPs of those VLPs are also within the scope of the present invention.

In a further preferred embodiment of the present invention, the antigen or antigenic determinant is a prion protein.

In another preferred embodiment of the invention, the antigen or antigenic determinant comprises or is a prion protein dimer. Two prion protein monomers may be brought together by linkage of the C-terminus of one prion protein monomer with the N-terminus of another prion protein monomer via an amino acid linker. Amino acid linkers suitable for dimerizing two prion protein monomers have been described in the Art, such for example by Dolezal O., et al., Protein Eng. 13: 565-74 (2000), or Perisic O., et al, Structure 2: 1217-26 (1994)). The more preferred length of the described Glycine-Serine amino acid linkers is of 1 to 10 amino acids, preferably 3-10 amino acids, and even more preferably 3-5 amino acids.

Dimerization of two prion protein monomers may also be carried out by attaching a dimerization domain to the prion protein monomer, for example the human IgG1 CH3 domain (Hu S., et al., Cancer Res. 56: 3055-61 (1996)) at the C-terminus of said prion protein monomer. Attachment of the dimerization domain to the C-terminus of the prion protein monomer is preferably mediated by an amino acid linker or connecting peptide, as described (Hu S., et al., Cancer Res. 56: 3055-61 (1996)).

Further dimerization domains, which may be attached at the N- or C-terminus of the prion protein monomer, via an amino acid linker or connecting peptide have been described by De Kruif et al., J. Biol. Chem. 271: 7630-34 (1996). The authors describe the use of Fos and Jun Leucine zippers for the dimerization of a scFv antibody fragment. The dimerization domain is attached to the C-terminus of the scFv fragment, via an amino acid linker of sequence: PKPSTPPGSSCGG (SEQ ID NO: 111). The sequence GGC is added at the C-terminus of both the FOS and Jun helix for improved dimerization. Likewise, one Jun helix and one Fos helix is attached at the C-terminus of each prion protein monomer to ensure dimer formation, with the sequence GGC at the C-terminus of each helix. When attaching the dimerization domains to the N-terminus of the prion protein, the amino acid linker is attached at the C-terminus of both Jun and fos helices, immediately at the N-terminus of each of the prion protein monomers. The sequence CGG is attached to the N-terminus of both Jun and Fos helices.

Further hetero-dimerization domains based on two alpha helices have been described by Chang et al., Proc. Natl. Acad. Sci. USA 91: 11408-12 (1994). The helical domains are called the acidic and basic peptide, respectively. The authors describe constructs where the helices are attached to the C-terminus of T-cell receptor α- and β-chains, via an amino acid linker sequence called “Linker” in the publication. Thus, as described above, the acid and basic peptides are attached as dimerization domain either at the N- or C-terminus of the prion protein monomer, such attachment being mediated by an amino acid linker or connecting peptide.

Hoyne et al., FEBS Lett. 479: 15-18 (2000) describe the use of the GCN4 leucine zipper of Saccharomyces cerevisiae as homo-dimerization domain. The GCN4 leucine zipper is fused to the N- or C-terminus of the prion protein monomer according to the guidance provided above.

When a dimerization domain is fused to the C-terminus of the prion protein monomer, an amino acid linker containing a cysteine residue as second attachment site is fused to the C-terminus of the dimerization domain, or to the N-terminus of the prion protein monomer, for binding of the prion protein to the VLP. When a dimerization domain is fused to the N-terminus of the prion protein monomer, an amino acid linker containing a cysteine residue as second attachment site is attached to the N-terminus of the dimerization domain, or to the C-terminus of the prion protein monomer for binding to the VLP.

In a further very preferred embodiment of the invention, the antigen or antigenic determinant is selected form the group consisting of a) human prion protein; b) bovine prion protein; c) sheep prion protein; d) elk prion protein; e) mule deer prion protein; f) white-tailed deer prion protein; g) pig prion protein; h) chicken prion protein; i) mouse prion protein; j) goat prion protein; and k) a peptide or a fragment thereof of any prion protein of a)-j).

In a further very preferred embodiment of the invention, the antigen or antigenic determinant comprises, alternatively essentially consists of, or alternatively consists of an amino acid sequence selected from the group consisting a) the amino acid sequence of SEQ ID NO: 79; b) the amino acid sequence of SEQ ID NO: 80; c) the amino acid sequence of SEQ ID NO: 81; d) the amino acid sequence of SEQ ID NO: 82; e) the amino acid sequence of SEQ ID NO: 83; f) the amino acid sequence of SEQ ID NO: 84; g) the amino acid sequence of SEQ ID NO: 85; h) the amino acid sequence of SEQ ID NO: 86; i) the amino acid sequence of SEQ ID NO: 87; j) the amino acid sequence of SEQ ID NO: 88; and k) the amino acid sequence of any fragment of any of SEQ ID NO:79-88.

In a further preferred embodiment of the invention, the antigen or antigenic determinant is a PrP protein variant, e.g. containing amino acid substitutions or peptide insertions or polymorphisms. As already indicated, compositions and vaccine compositions, respectively, comprising PrP protein variants are included within the scope of the present invention. A list of preferred examples including but not limited to such PrP variants, in particular to human PrP variants is given in Table 1.

TABLE 1
SELECTED HUMAN PRP VARIANTS

| Pathogenic human Poly- | |
|---|---|
| morphisms Mutations | Reference |
| Octarepeat inserts: | |
| 1-9 inserts | Palmer, M. S. and Collinge J., Hum. |
| | Mutat. 2:168-73 (1993); Owen, F., |
| | Brain Res. Mol. Brain Res. 7:273-276 |
| | (1990); Krasemann S., et al., Brain |
| | Res Mol Brain Res 34:173-176 |
| | (1995); Goldfarb, L, et al., PNAS |
| | 88:10926-301991 (1191). |
| Aminoacid substitutions: | |
| Pro102Leu (Gerstmann- | Barbanti, P., et al., Neurology |
| Straussler sydrome (GSS)) | 47:734-741(1996) |
| Ala117Val (GSS) | Doh-ura, K. et al., Biochem. Biophys. |
| | Res. Commun. 163:974-979 (1989) |
| Asp178Asn (Fatal | Medori, R., et al., Neurology |
| familial insomnia) | 42:669-670 (1992) |
| Val180Ile (Creutzfeldt-Jakob | Kitamoto, T., et al., Biochem. |
| disease (CJD) | Biophys. Res. Commun. 191:709-714 |
| | (1993) |
| Phe198Ser (GSS with | Hsiao K, et al., Nat Genet. 1:68-71 |
| neurofibrillary tangles) | (1992) |
| Glu200Lys (CJD) | Inoue, I., et al. Neurology |
| | 44:299-301 (1994) |
| Val210Ile (CJD) | Pocchiari, M., et al., Ann. Neurol. |
| | 34:802-807 (1993) |
| Asn232Arg (CJD) | Kitamoto, T., et al., Biochem. |
| | Biophys. Res. Commun. |
| | 191:709-714 (1993) |
| Human polymorphisms: | |
| Met129Val | Doh-ura, K. et al., Biochem. Biophys. |
| | Res. Commun. 163:974-979 (1989) |
| Gly219Lys | Barbanti, P., et al., Neurology |
| | 47:734-741 (1996). |

In a further very preferred embodiment of the invention, the antigen or antigenic determinant is the human, sheep or bovine prion protein.

Prion proteins can be produced by expression of the PrP cDNA in procaryotic or eucaryotic expression systems. Various examples hereto have been described in the literature and can be used, possibly after modifications, to express any prion protein of any desired species. For example as described by Volkel et al. (Volkel, D., Eur J Biochem 251:462-471 (1998)) for E. coli, expression of Syrian golden hamster PrP (residues 23-231) was achieved by C-terminally fusing Syrian golden hamster PrP to glutathione S-transferase (GST), spaced by a thrombin cleavage site and by N-terminally fusing a histidine tag, using standard molecular biological techniques. Expression of this gene construct, followed by refolding, removal of GST and purification, lead to 50-100 mg homogeneous protein/1 bacterial culture. In .a similar approach sheep PrP was produced in large quantities in Escherichia coli after fusion with a carboxy-terminal hexahistidine sequence as described by Baron et. al. (Baron., T., et al., FEMS Immunol Med Microbiol 25:379-384 (1999)). Other tags such as the FLAG tag, the HA tag or the constant region of an antibody (Fc region) may also be used for purification. For eucaryotic expression overexpression of Syrian golden hamster PrP has been achieved using cleavable glutathione S-transferase fusions. (Weiss, S., J. Virol. 69:4776-4783 (1995)). Expression was achieved with baculovirus-infected insect cells (and with Escherichia coli) and PrP was released from the immobilized fusion protein by direct cleavage with thrombin. In a further approach Syrian hamster PrP expression was obtained in Chinese hamster ovary (CHO) cells using a glutamine synthetase selection and amplification system (Blochberger T., et al., Protein Eng D 10(12):1465-73 (1997)). A cell clone that expressed the highest levels of full length PrP was subcloned for isolation and analysis of PrP. In yet a further experimental set up, a tetracycline-inducible expression system was established for high level expression of mouse PrP in murine N2a neuroblastoma cells using standard molecular and cell biological methods. N2a clones were fully controllable with respect to expression of PrP, as over-expression could be switched on and off as desired using the tetracycline-derivative doxycycline as an effector substance (Windl, O., et al., J. Gen. Virol. 80:15-21, (1999)).

In a further preferred embodiment of the invention, the antigen or antigenic determinant is a PrP peptide or a fragment thereof. Preferably the prion peptide is selected from the group consisting: a) human prion peptide; b) bovine prion peptide; c) sheep prion peptide; d) elk prion peptide; e) mule deer prion peptide; f) white-tailed deer prion peptide; g) pig prion peptide; h) chicken prion peptide; i) mouse prion peptide; j) goat prion peptide; and k) a fragment of any prion peptide of a)-j).

In another prefered embodiment of the invention, the prion protein fragment is a construct comprising the globular domain extending from residues 125 to 228 of the human prion protein or other mammalian homologues, described by Zahn et al., Proc. Natl. Acad. Sci. USA 97: 145-150 (2000). Such constructs include the fragments 23-230, 121-230 and 90-230, all modified with a C-terminal or N-terminal amino acid linker containing a cysteine residue as second attachment site. The fragments may be expressed and purified as described (Zahn et al., Proc. Natl. Acad. Sci. USA 97: 145-150 (2000)), or the cDNA encoding these fragments may be cloned as set out in Example 13 in the pCEP-SP-EK-Fc* vector and expressed and purified as described in Example 13. Alternatively, the constructs may be cloned in pMod vectors as described in WO/02056905 filed by the present assignee.

PrP peptides or fragments thereof can be produced using standard molecular biological technologies where the nucleotide sequence coding for the fragment of interest is amplified by PCR and is cloned as a fusion to a polypeptide tag, such as the histdine tag, the Flag tag, myc tag or the constant region of an antibody (Fc region). By introducing an enterokinase cleavage site between the PrP fragment and the tag, the PrP fragment can be separated from the tag after purification by digestion with enterokinase. In another approach the PrP fragment can be syntesized in vitro using standard peptide synthesis reactions known to a person skilled in the art. In a further approach PrP fragments can be produced by protease digestion or chemical cleavage of the full length prion protein, both methods which are well known to people trained in the art. In yet a further approach, Corsaro et al. describe a procedure, which allows the expression and purification of the native form of a human prion protein fragment 90-231, corresponding to the protease resistant core of PrPsc. Using a glutathione S-transferase (GST) fusion protein approach, milligram amounts of polypeptides were obtained after expression in E. coli. The PrP fragment was released from the fusion protein immobilized on a glutathione-coupled agarose resin by direct cleavage with thrombin (Corsaro, A., et al., Neurochem. Int. 41:55-63 (2002)).

In a further preferred embodiment of the present invention, the prion peptide comprises at least one antigenic site of a prion protein. The skilled person in the art knows how to identify the corresponding peptides and amino acid sequences, respectively. Several prion peptides having at least one antigenic site of a prion protein and being suitable for use in the present invention are described in WO 93/11155, the entirety of which is explicitly incorporated by reference. In particular, six regions defined by general formulas providing amino acid sequences of synthetic peptides are reported. One of these regions was shown to harbor two additional frame shift mutations resulting in additional amino acid sequences. Typical and preferred examples for human PrP peptides useful for the present invention include but are not limited to the following human PrP peptides that are the indicated fragments of SEQ ID NO: 79 as as well as sub-fragments thereof:

| | | |
|---|---|---|
| HU PRP | 109-139: | MKHMAGAAAAGAVVGGLGGYMLGSAMSRPII |
| HU PRP | 132-160: | SAMSRPIIHFGSDYEDRYYRENMHRYPNQ |
| HU PRP | 153-181: | NMHRYPNQVYYRPMDEYSNQNNFVHDCVN |
| HU PRP | 225-253: | YYQRGSSMVLFSSPPVILLISFLIFLIVG |
| HU PRP | 54-69: | GGGWGQPHGGGWGQPH |
| HU PRP | 174-204: | NFVHDCVNITIKQHTVTTTTKGENFTETDVK |
| HU PRP | 211-232: | EQMCITQYERESQAYYQRGSSM |

Guidance on how to modify human prion protein, prion peptide or a fragment thereof, in particular, for binding to the virus-like particle is given throughout the application and in particular in Example 13. Mouse prion protein constructs are disclosed, and a preferred human prion protein constructs, i.e. a human prion peptide, can also be generated and have, for example, the sequence of SEQ ID NO: 89. Further constructs comprise the whole human prion protein sequence and fragments thereof, Immunization against prion protein using the inventive compositions comprising, preferably a human prion protein or human prion peptide bound to a core particle and VLP, respectively, may provide a way of treatment or prevention of Creutzfeldt-Jakob (variant form) or other prion diseases. In the former case, this corresponds to an immunization against a self-antigen. Immunization using the compositions of the invention comprising the prion protein or prion peptide may provide a way of treatment against prion mediated diseases in other animals as well. Preferred embodiments, thus, include the corresponding sequences of bovine and sheep prion protein constructs, which are given in SEQ ID NO:90 and SEQ ID NO:91. However, the corresponding amino acid sequences of the elk prion peptide, mule and white-tailed deer are also preferred embodiments of the invention.

In a further very preferred embodiment of the present invention, the antigen or antigenic determinant is a prion peptide comprising, or alternatively essentially consisting of, or alternatively consisiting of an amino acid sequence selected from the group consisting of:

(a) SAMSRPIIHFGSDYEDRYYRENMHR (“human prplong”) (SEQ ID NO: 112);

(b) GSDYEDRYYRENMHR (“human prpshort”) (SEQ ID NO: 113);

(c) SAMSRPLIHFGSDYEDRYYRENMHR (“bovine prplong”) (SEQ ID NO: 114);

(d) GSDYEDRYYRENMHR (“bovine prpshort”) (SEQ ID NO: 115);

(e) SAMSRPLIHFGNDYEDRYYRENMYR (“sheep prplong”) (SEQ ID NO: 116);

(f) GNDYEDRYYRENMYR (“sheep prpshort”) (SEQ ID NO: 117);

(g) SAMNRPLIHFGNDYEDRYYRENMYR (“deer prplong”)(SEQ ID NO: 118);

(h) GNDYEDRYYRENMYR (“deer prpshort”) (SEQ ID NO: 119);

(i) SAMSRPLIHFGNDYEDRYYRENMYR (“elk prplong”)(SEQ ID NO: 120);

(1) GNDYEDRYYRENMYR (“elk prpshort”) (SEQ ID NO: 121); and

(k) the amino acid sequence of any fragment of any of (a)-(j).

In a still further preferred embodiment of the present invention, the antigen or antigenic determinant further comprise at least one second attachment site being selected from the group consisting of: (i) an attachment site not naturally occurring with said antigen or antigenic determinant; and (ii) an attachment site naturally occurring with said antigen or antigenic determinant. Preferably, the antigen or antigenic determinant with said at least second attachment site comprises, or alternatively consists of, or alternatively consists of an amino acid sequence selected from the group consisting of:

(a) CSAMSRPIIHFGSDYEDRYYRENMHR (“human cprplong”) (SEQ ID NO: 122);

(b) CGSDYEDRYYRENMHR (“human cprpshort”) (SEQ ID NO: 123);

(c) CSAMSRPLIHFGSDYEDRYYRENMHR (“bovine cprplong”) (SEQ ID NO: 124);

(d) CGSDYEDRYYRENMHR (“bovine cprpshort”) (SEQ ID NO: 125);

(e) CSAMSRPLIHFGNDYEDRYYRENMYR (“sheep cprplong”) (SEQ ID NO: 126);

(f) CGNDYEDRYYRENMYR (“sheep cprpshort”) (SEQ ID NO: 127);

(g) CSAMNRPLHIFGNDYEDRYYRENMYR (“deer cprplong”) (SEQ ID NO: 128);

(h) CGNDYEDRYYRENMYR (“deer cprpshort”) (SEQ ID NO: 129);

(i) CSAMSRPLIHFGNDYEDRYYRENMYR (“elk cprplong”) (SEQ ID NO: 130);

(l) CGNDYEDRYYRENMYR (“elk cprpshort”) (SEQ ID NO: 131); and

(k) the amino acid sequence of any fragment of any of (a)-(j).

The corresponding murine peptides are described in Example 14. These peptides comprise an N-terminal cysteine residue as a second attachment added for coupling to VLPs and Pili.

Further PrP peptides suitable for usein the present invention can be experimentally determined by their intrinsic property to induce a T cell or an antibody response. This is generally achieved by immunizing an experimental animal separately with selected peptides in an immunologically suitable formulation and by measuring T cell and B cell, i.e. antibody responses, using methods known to a person trained in the art. Suoan et al. immunized mice with a panel of overlapping mouse PrP peptides, in toto covering the full length mouse PrP protein and measured T cell and B cell responses (Souan L., et al., Eur. J. Immunol. 31:2338-2346, (2001)). Prion peptides capable of inducing strong T cell and/or B cell responses have been identified. Thus, preferred embodiments of the present invention are mouse PrP peptides comprising, or alternatively essentially consisting of, or alternatively consisting of an amino acid sequence selected from (a) SAMSRPMIHFGNDWEDRYYR (Mouse PrP, SEQ ID NO: 87, amino acids 131-150), (b) QMCVTQYQKESQAYYDGRRS (Mouse PrP, SEQ ID NO: 87, amino acids 211-230), and the corresponding amino acid sequences of (a) or (b) of human, bovine, sheep, elk, mule deer, white-tailed deer, pig, chicken , or goat (SEQ ID NOs: 79 through 93, inclusive, inter alia) as well as the amino acid sequence of any fragment of any indicated species and animal, respectively, of (a) or (b).

According to the prion hypothesis, prion infectivity resides in PrP^{SC} which is a conformational variant of a host encoded cellular protein designated PrP^{C}. Further prion peptides suitable for the present invention are peptides which contain at least one antibody epitope of PrP^{C} or PrP^{SC}. In the case where the antigen is a protein, a polypeptide or a peptide, this region can be formed by a continuous aminoacid sequence. Alternatively, the antibody epitope can be formed by a discontinuous aminoacid sequence in which, after three dimensional folding of the protein, polypeptide or peptide, the aminoacids are arranged in such a manner that they spatially come close together and form the epitope. Continuous peptide fragments of interest can identified by immunization experiments as described above.

Considering conversion of PrP^{C} to PrP^{SC} involves a major change of protein conformation, it is likely that unique epitopes are formed or revealed upon conversion. Thus, a so-called side-chain hypothesis pertaining to PrP conversion has been formulated. According to this hypothesis side chains normally sequestered in the solvent-inaccessible interior of PrP^{C} may be solvent accessible in PrP^{SC}. The extrusion of the hydrophobic side chains form the basis of unique epitopes for antibody recognition of PrP^{SC}. Based on this hypothesis synthetic peptides containing at least one YYX (YYR, YYQ or YYD) epitope of PrP have been identified in WO00/78344 and were characterized by general formulae. These peptides were chosen on the basis of in vitro experiments and molecular modeling analyses and were claimed to induce high-affinity monoclonal and polyclonal anitbodies specific for PrP^{SC}.

Further preferred PrP peptides suitable for use for the present invention have an amino acid sequence, which comprises, or alternatively essentially consists of, or alternatively consists of an amino acid sequence selected from CYYR (SEQ ID NO: 132), CYYRRYYRRYY (SEQ ID NO: 133), AYYQ (SEQ ID NO: 134), and AYYQYYQYYQ (SEQ ID NO: 135).

Peptide fragments suitable for use as antigen or antigenic determinant for the present invention forming an epitope on the surface of PrP^{SC} and capable of binding PrP-specific antibodies were identified in mapping experiments using immobilized peptide banks (WO/9915651). These peptide banks (available from Jerini Biotools, Berlin) cover the full length PrP protein sequence and were screened against a monoclonal antibody named 15B3 specifically binding PrP^{SC}. Again, this systematic approach defined general formulae defining PrP petides suitable for use for the present invention. Further preferred PrP peptides suitable for use for the present invention have an amino acid sequence, which comprises, or alternatively essentially consists of, or alternatively consists of an amino acid sequence selected from (a) GSDYEDRYY (Human PrP, SEQ ID NO: 79, amino acids 142-150), (b) GNDYEDRYY (Sheep PrP, SEQ ID NO: 81, amino acids 145-153), and the corresponding amino acid sequences of (a) or (b) of human, bovine, sheep, elk, mule deer, white-tailed deer, pig, chicken or goat (inter alia, SEQ ID NOs: 79 through 93, inclusive) as well as the amino acid sequence of any fragment of any indicated species and animal, respectively, of (a) or (b).

Further preferred prion peptides suitable for use for the present invention can be identified by using existing or future monoclonal or polyclonal antibodies.

Such antibodies may include for example the monoclonal antibodies 6H4 and 34C9 (WO 98/3721; EP 861'900, Heppner et al. Science 294:178-182, (2001)) recognizing bovine PrP peptide 155-163 and bovine PrP peptide 149-153, respectively. Similarly, PrP peptides or PrP fragments recognized by antibodies recognizing the same or equivalent or variant or distinct epitopes in any species and which may be able to prevent or delay the onset of prion disease are preferred embodiments of the present invention. U.S. Pat. No. 5,688,651 teaches a method for the selection of such antibodies.

Further PrP peptides suitable for use for the present invention can be defined by other antibodies including monoclonal antibodies mAb 3-11 or mAb 2-40, both recognizing human PrP peptide 106-126, or related antibodies which inhibit PrP peptide neurotoxicity or fibril formation (Forloni, G., et. al. Nature 362:543-546, (1993); Brown, D. R., Glia 18:59-67 (1996)). Yet other PrP peptides suitable for use for the present invention are defined by antibodies including monoclonal antibody CNCM 1-2476 which is capable of selectively binding the three dimensional conformation provided by the C-terminal part of the PrP^{Sc} isoform of the prion protein or part thereof, while not binding to the PrP^{C} form (WO 01/90191).

In a further preferred embodiment of the invention, the antigen or antigenic determinant is a prion peptide having an amino acid sequence, which comprises, or alternatively essentially consists of, or alternatively consists of an amino acid sequence selected from the (SEQ ID NO: 79) human PrP sequence, amino acids 214-226: CITQYERESQAYY or variants thereof; or corresponding sequences in other species, for example (SEQ ID NO: 80) bovine PrP peptide amino acids 225-237 CITQYQRESQAYY or variants thereof.

Further PrP peptides suitable for use for the present invention may be identified by screening phage display peptide libraries with antibodies specific for PrP^{SC} or PrP^{C}, a method well known to a person trained in the art. Furthermore, PrP peptides which have fusogenic and fibril formation properties and therefore are neurotoxic (Pillot, T. J., Mol. Biol. 5:381-393 (1997)) are further preferred embodiments suitable for use for the present invention.

In a further preferred embodiment of the invention, the antigen or antigenic determinant is isolated or recombinant PrP2 of any animal as well as any antigenic fragments of PrP2 of any animal. PrP2 represents mammalian -prion-like compositions identified in part on the basis of homology of the cDNA encoding for PrP2 to PrP of various species as outlined in WO 01/46419.

### EXAMPLE 1

### Construction and Expression of Mutant Qβ Coat Proteins, and Purification of Mutant Qβ Coat Protein VLPs or Capsids

### Plasmid Construction and Cloning of Mutant Coat Proteins

Construction of pQβ-240:

The plasmid pQβ10 (Kozlovska, T M, et al., Gene 137:133-137) was used as an initial plasmid for the construction of pQβ-240. The mutation Lys13→Arg was created by inverse PCR. The inverse primers were designed in inverted tail-to-tail directions:

5′-GGTAACATCGGTCGAGATGGAAAACAAACTCTGGTCC-3′ (SEQ ID NO: 136) and

5′-GGACCAGAGTTTGTTTTCCATCTCGACCGATGTTACC-3′ (SEQ ID NO: 137).

The products of the first PCR were used as templates for the second PCR reaction, in which an upstream primer

5′-AGCTCGCCCGGGGATCCTCTAG-3′ (SEQ ID NO:138) and a downstream primer

5′-CGATGCATTTCATCCTTAGTTATCAATACGCTGGGTTCAG-3′ (SEQ ID NO:139)

were used. The product of the second PCR was digested with XbaI and Mph1103I and cloned into the pQβ10 expression vector, which was cleaved by the same restriction enzymes. The PCR reactions were performed with PCR kit reagents and according to producer protocol (MBI Fermentas, Vilnius, Lithuania).

Sequencing using the direct label incorporation method verified the desired mutations. E. coli cells harbouring pQβ-240 supported efficient synthesis of 14-kD protein co migrating upon SDS-PAGE with control Qβ coat protein isolated from Qβ phage particles.

Resulting Amino Acid Sequence: (SEQ ID NO: 23)

AKLETVTLGNIGRDGKQTLVLNPRGVNPTNGVASLSQAGAVPALEKRVTV
SVSQPSRNRKNYKVQVKIQNPTACTANGSCDPSVTRQKYADVTFSFTQYS
TDEERAFVRTELAALLASPLLIDAIDQLNPAY

Construction of pQβ-243:

The plasmid pQβ10 was used as an initial plasmid for the construction of pQβ-243.The mutation Asn10 →Lys was created by inverse PCR. The inverse primers were designed in inverted tail-to-tail directions:

5′-GGCAAAATTAGAGACTGTTACTTTAGGTAAGATCGG-3′ (SEQ ID NO: 140) and

5′-CCGATCTTACCTAAAGTAACAGTCTCTAATTTTGCC-3′ (SEQ ID NO: 141).

The products of the first PCR were used as templates for the second PCR reaction, in which an upstream primer

5′-AGCTCGCCCGGGGATCCTCTAG-3′ (SEQ ID NO:138)

and a downstream primer

5′-CGATGCATTTCATCCTTAGTTATCAATACGCTGGGTTCAG-3′ (SEQ ID NO: 139)

were used. The product of the second PCR was digested with XbaI and Mph1103I and cloned into the pQβ10 expression vector, which was cleaved by the same restriction enzymes. The PCR reactions were performed with PCR kit reagents and according to producer protocol (MBI Fermentas, Vilnius, Lithuania).

Sequencing using the direct label incorporation method verified the desired mutations. E. coli cells harbouring pQβ-243 supported efficient synthesis of 14-kD protein co migrating upon SDSD-PAGE with control Qβ coat protein isolated from Qβ phage particles.

Resulting Amino Acid Sequence: (SEQ ID NO: 24)

AKLETVTLGKIGKDGKQTLVLNPRGVNPTNGVASLSQAGAVPALEKRVTV
SVSQPSRNRKNYKVQVKIQNPTACTANGSCDPSVTRQKYADVTFSFTQYS
TDEERAFVRTELAALLASPLLIDAIDQLNPAY

Construction of pQβ-250:

The plasmid pQβ-240 was used as an initial plasmid for the construction of pQβ-250. The mutation Lys2→Arg was created by site-directed mutagenesis. An upstream primer

5′-GGCCATGGCACGACTCGAGACTGTTACTTTAGG-3′ (SEQ ID NO: 142)

and a downstream primer

5′-GATTTAGGTGACACTATAG-3′ (SEQ ID NO: 143)

were used for the synthesis of the mutant PCR-fragment, which was introduced into the pQβ-185 expression vector at the unique restriction sites NcoI and HindIII. The PCR reactions were performed with PCR kit reagents and according to producer protocol (MBI Fermentas, Vilnius, Lithuania).

Sequencing using the direct label incorporation method verified the desired mutations. E. coli cells harbouring pQβ-250 supported efficient synthesis of 14-kD protein co migrating upon PAGE with control Qβ coat protein isolated from Qβ phage particles.

Resulting Amino Acid Sequence: (SEQ ID NO: 25)

ARLETVTLGNIGRDGKQTLVLNPRGVNPTNGVASLSQAGAVPALEKRVTV
SVSQPSRNRKNYKVQVKIQNPTACTANGSCDPSVTRQKYADVTFSFTQYS
TDEERAFVRTELAALLASPLLIDAIQLNPAY

Construction of pQβ-251:

The plasmid pQβ10 was used as an initial plasmid for the construction of pQβ-251. The mutation Lys16→Arg was created by inverse PCR. The inverse primers were designed in inverted tail-to-tail directions:

5′-GATGGACGTCAAACTCTGGTCCTCAATCCGCGTGGGG -3′ (SEQ ID NO: 144)

and

5′-CCCCACGCGGATTGAGGACCAGAGTTTGACGTCCATC -3′ (SEQ ID NO: 145).

The products of the first PCR were used as templates for the second PCR reaction, in which an upstream primer

5′-AGCTCGCCCGGGGATCCTCTAG-3′ (SEQ ID NO: 138)

and a downstream primer

5′-CGATGCATTTCATCCTTAGTTATCAATACGCTGGGTTCAG-3′ (SEQ ID NO: 139)

were used. The product of the second PCR was digested with XbaI and Mph1103I and cloned into the pQβ10 expression vector, which was cleaved by the same restriction enzymes. The PCR reactions were performed with PCR kit reagents and according to producer protocol (MBI Fermentas, Vilnius, Lithuania).

Sequencing using the direct label incorporation method verified the desired mutations. E. coli cells harbouring pQβ-251 supported efficient synthesis of 14-kD protein co migrating upon SDS-PAGE with control Qβ coat protein isolated from Qβ phage particles. The resulting amino acid sequence encoded by this construct is shown in SEQ. ID NO: 26.

Construction of pQβ-259:

The plasmid pQβ-251 was used as an initial plasmid for the construction of pQβ-259. The mutation Lys2→Arg was created by site-directed mutagenesis.

An Upstream Primer

5′-GGCCATGGCACGACTCGAGACTGTTACTTTAGG-3′ (SEQ ID NO: 142)

and a downstream primer

5′-GATTTAGGTGACACTATAG-3′ (SEQ ID NO: 143)

were used for the synthesis of the mutant PCR-fragment, which was introduced into the pQβ-185 expression vector at the unique restriction sites NcoI and HindIII. The PCR reactions were performed with PCR kit reagents and according to producer protocol (MBI Fermentas, Vilnius, Lithuania).

Sequencing using the direct label incorporation method verified the desired mutations. E. coli cells harbouring pQβ-259 supported efficient synthesis of 14-kD protein co migrating upon SDS-PAGE with control Qβ coat protein isolated from Qβ phage particles.

Resulting Amino Acid Sequence: (SEQ ID NO: 27)

AKLETVTLGNIGKDGKQTLVLNPRGVNPTNGVASLSQAGAVP
ALEKRVTVSVSQPSRNRKNYKVQVKIQNPTACTANGSCDPSVTRQ
KYADVTFSFTQYSTDEERAFVRTELAALLASPLLIDAIDQLNPAY

General Procedures for Expression and Purification of Qβ and Qβ Mutants

Expression

E. coli JM109 was transformed with Qβ coat protein expression plasmids. 5 ml of LB liquid medium containing 20 μg/ml ampicillin were inoculated with clones transformed with with Qβ coat protein expression plasmids. The inoculated culture was incubated at 37° C. for 16-24 h without shaking. The prepared inoculum was subsequently diluted 1:100 in 100-300 ml of fresh LB medium, containing 20 μg/ml ampicillin. and incubated at 37° C. overnight without shaking. The resulting second inoculum was diluted 1:50 in M9 medium containing 1% Casamino acids and 0.2% glucose in flasks, and incubated at 37° C. overnight under shaking.

Purification

Solutions and buffers for the purification procedure:

1. Lysis buffer LB

50 mM Tris-HCl pH 8,0 with 5 mM EDTA, 0,1%

tritonX100 and freshly prepared PMSF at a concentration of 5micrograms per

ml. Without lysozyme and DNAse.

2. SAS

Saturated ammonium sulphate in water

3. Buffer NET.

20 mM Tris-HCl, pH 7.8 with 5 mM EDTA and

150 mM NaCl.

4. PEG

40% (w/v) polyethylenglycol 6000 in NET

Disruption and Lysis

Frozen cells were resuspended in LB at 2 ml/g cells. The mixture was sonicated with 22 kH five times for 15 seconds, with intervals of 1 min to cool the solution on ice. The lysate was then centrifuged at 14 000 rpm, for 1h using a Janecki K 60 rotor. The centrifugation steps described below were all performed using the same rotor, except otherwise stated. The supernatant was stored at 4° C., while cell debris were washed twice with LB. After centrifugation, the supernatants of the lysate and wash fractions were pooled.

Fractionation

A saturated ammonium sulphate solution was added dropwise under stirring to the above pooled lysate. The volume of the SAS was adjusted to be one fifth of total volume, to obtain 20% of saturation. The solution was left standing overnight, and was centrifuged the next day at 14 000 rpm, for 20 min. The pellet was washed with a small amount of 20% ammonium sulphate, and centrifuged again . The obtained supernatants were pooled, and SAS was added dropwise to obtain 40% of saturation. The solution was left standing overnight, and was centrifuged the next day at 14 000 rpm, for 20 min. The obtained pellet was solubilised in NET buffer.

Chromatography

The capsid or VLP protein resolubilized in NET buffer was loaded on a Sepharose CL-4B column. Three peaks eluted during chromatography. The first one mainly contained membranes and membrane fragments, and was not collected. Capsids were contained in the second peak, while the third one contained other E. coli proteins.

The peak fractions were pooled, and the NaCl concentration was adjusted to a final concentration of 0.65 M. A volume of PEG solution corresponding to one half of the pooled peak fraction was added dropwise under stirring. The solution was left to stand overnight without stirring. The capsid protein was sedimented by centrifugation at 14 000 rpm for 20 min. It was then solubilized in a minimal volume of NET and loaded again on the Sepharose CL-4B column. The peak fractions were pooled, and precipitated with ammonium sulphate at 60% of saturation (w/v). After centrifugation and resolubilization in NET buffer, capsid protein was loaded on a Sepharose CL-6B column for rechromatography.

Dialysis and Drying

The peak fractions obtained above were pooled and extensively dialysed against sterile water, and lyophilized for storage.

Expression and Purification Qβ-240

Cells (E. coli JM 109, transformed with the plasmid pQβ-240) were resuspended in LB, sonicated five times for 15 seconds (water ice jacket) and centrifuged at 13000 rpm for one hour. The supernatant was stored at 4° C. until further processing, while the debris were washed 2 times with 9 ml of LB, and finally with 9 ml of 0,7 M urea in LB. All supernatants were pooled, and loaded on the Sepharose CL-4B column. The pooled peak fractions were precipitated with ammonium sulphate and centrifuged. The resolubilized protein was then purified further on a Sepharose 2B column and finally on a Sepharose 6B column. The capsid peak was finally extensively dialyzed against water and lyophilized as described above. The assembly of the coat protein into a capsid was confirmed by electron microscopy.

Expression and Purification Qβ-243

Cells (E. coli RR1) were resuspended in LB and processed as described in the general procedure. The protein was purified by two successive gel filtration steps on the sepharose CL-4B column and finally on a sepharose CL-2B column. Peak fractions were pooled and lyophilized as described above. The assembly of the coat protein into a capsid was confirmed by electron microscopy.

Expression and Purification of Qβ-250

Cells (E. coli JM 109, transformed with pQβ-250) were resuspended in LB and processed as described above. The protein was purified by gel filtration on a Sepharose CL-4B and finally on a Sepharose CL-2B column, and lyophilized as described above. The assembly of the coat protein into a capsid was confirmed by electron microscopy.

Expression and Purification of Qβ-259

Cells (E. coli JM 109, transformed with pQβ-259) were resuspended in LB and sonicated. The debris were washed once with 10 ml of LB and a second time with 10 ml of 0,7 M urea in LB. The protein was purified by two gel-filtration chromatogaphy steps, on a Sepharose CL-4 B column. The protein was dialyzed and lyophilized, as described above. The assembly of the coat protein into a capsid was confirmed by electron microscopy.

### EXAMPLE 2

### Insertion of a Peptide Containing a Lysine Residue Into the c/e1 Epitope of HBcAg(1-149).

The c/e1 epitope (residues 72 to 88) of HBcAg is located in the tip region on the surface of the Hepatitis B virus capsid (HBcAg). A part of this region (Proline 79 and Alanine 80) was genetically replaced by the peptide Gly-Gly-Lys-Gly-Gly (HBcAg-Lys construct) (SEQ ID NO: 78). The introduced Lysine residue contains a reactive amino group in its side chain that can be used for intermolecular chemical crosslinking of HBcAg particles with any antigen containing a free cysteine group.

HBcAg-Lys DNA, having the amino acid sequence shown in SEQ ID NO:78, was generated by PCRs: The two fragments encoding HBcAg fragments (amino acid residues 1 to 78 and 81 to 149) were amplified separately by PCR. The primers used for these PCRs also introduced a DNA sequence encoding the Gly-Gly-Lys-Gly-Gly peptide. The HBcAg (1 to 78) fragment was amplified from pEco63 using primers EcoRIHBcAg(s) and Lys-HBcAg(as). The HBcAg (81 to 149) fragment was amplified from pEco63 using primers Lys-HBcAg(s) and HBcAg(1-149)Hind(as). Primers Lys-HBcAg(as) and Lys-HBcAg(s) introduced complementary DNA sequences at the ends of the two PCR products allowing fusion of the two PCR products in a subsequent assembly PCR. The assembled fragments were amplified by PCR using primers EcoRIHBcAg(s) and HbcAg(1-149)Hind(as).

For the PCRs, 100 pmol of each oligo and 50 ng of the template DNAs were used in the 50 ml reaction mixtures with 2 units of Pwo polymerase, 0.1 mM dNTPs and 2 mM MgSO4. For both reactions, temperature cycling was carried out as follows: 94° C. for 2 minutes; 30 cycles of 94° C. (1 minute), 50° C. (1 minute), 72° C. (2 minutes).

Primer Sequences:

| | |
|---|---|
| EcoRIHBcAg(s): | |
| (5′-CCGGAATTCATGGACATTGACCCTTATAAAG-3′); | (SEQ ID NO: 146) |
| Lys-HBcAg(as): | |
| (5′-CCTAGAGCCACCTTTGCCACCATCTTCTAAATTAGTACCCACCCAGGTAGC-3′); | (SEQ ID NO: 147) |
| Lys-HBcAg(s): | |
| (5′-GAAGATGGTGGCAAAGGTGGCTCTAGGGACCTAGTAGTCAGTTATGTC-3′); | (SEQ ID NO: 148) |
| HBcAg(1-149)Hind(as): | |
| (5′-CGCGTCCCAAGCTTCTAAACAACAGTAGTCTCCGGAAG-3′). | (SEQ ID NO: 149) |

For fusion of the two PCR fragments by PCR 100 pmol of primers EcoRIHBcAg(s) and HBcAg(1-149)Hind(as) were used with 106 ng of the two purified PCR fragments in a 50 ml reaction mixture containing 2 units of Pwo polymerase, 0.1 mM dNTPs and 2 mM MgSO₄. PCR cycling conditions were: 94° C. for 2 minutes; 30 cycles of 94° C. (1 minute), 50° C. (1 minute), 72° C. (2 minutes). The assembled PCR product was analyzed by agarose gel electrophoresis, purified and digested for 19 hours in an appropriate buffer with EcoRI and HindIII restriction enzymes. The digested DNA fragment was ligated into EcoRI/HindIII-digested pKK vector to generate pKK-HBcAg-Lys expression vector. Insertion of the PCR product into the vector was analyzed by EcoRI/HindIII restriction analysis and DNA sequencing of the insert.

### EXAMPLE 3

### Expression and Purification of HBcAg-Lys

E. coli strains K802 or JM109 were transformed with pKK-HBcAg-Lys. 1 ml of an overnight culture of bacteria was used to innoculate 100 ml of LB medium containing 100 μg/ml ampicillin. This culture was grown for 4 hours at 37° C. until an OD at 600 nm of approximately 0.8 was reached. Induction of the synthesis of HBcAg-Lys was performed by addition of IPTG to a final concentration of 1 mM. After induction, bacteria were further shaken at 37° C. for 4 hours. Bacteria were harvested by centrifugation at 5000×g for 15 minutes. The pellet was frozen at −80° C. The pellet was thawed and resuspended in bacteria lysis buffer (10 mM Na₂HPO₄, pH 7.0, 30 mM NaCl, 0.25% Tween-20, 10 mM EDTA) supplemented with 200 μ/g/ml lysozyme and 10 μl of Benzonase (Merck). Cells were incubated for 30 minutes at room temperature and disrupted by sonication. E. coli cells harboring pKK-HBcAg-Lys expression plasmid or a control plasmid were used for induction of HBcAg-Lys expression with IPTG. Prior to the addition of IPTG, a sample was removed from the bacteria culture carrying the pKK-HBcAg-Lys plasmid and from a culture carrying the control plasmid. Four hours after addition of IPTG, samples were again removed from the culture containing pKK-HBcAg-Lys and from the control culture. Protein expression was monitored by SDS-PAGE followed by Coomassie staining.

The lysate was then centrifuged for 30 minutes at 12,000×g in order to remove insoluble cell debris. The supernatant and the pellet were analyzed by Western blotting using a monoclonal antibody against HBcAg (YVS1841, purchased from Accurate Chemical and Scientific Corp., Westbury, N.Y., USA), indicating that a significant amount of HBcAg-Lys protein was soluble. Briefly, lysates from E. coli cells expressing HBcAg-Lys and from control cells were centrifuged at 14,000×g for 30 minutes. Supernatant (=soluble fraction) and pellet (=insoluble fraction) were separated and diluted with SDS sample buffer to equal volumes. Samples were analyzed by SDS-PAGE followed by Western blotting with anti-HBcAg monoclonal antibody YVS 1841.

The cleared cell lysate was used for step-gradient centrifugation using a sucrose step gradient consisting of a 4 ml 65% sucrose solution overlaid with 3 ml 15% sucrose solution followed by 4 ml of bacterial lysate. The sample was centrifuged for 3 hrs with 100,000×g at 4° C. After centrifugation, 1 ml fractions from the top of the gradient were collected and analyzed by SDS-PAGE followed by Coomassie staining. The HBcAg-Lys protein was detected by Coomassie staining.

The HBcAg-Lys protein was enriched at the interface between 15 and 65% sucrose indicating that it had formed a capsid particle. Most of the bacterial proteins remained in the sucrose-free upper layer of the gradient, therefore step-gradient centrifugation of the HBcAg-Lys particles led both to enrichment and to a partial purification of the particles.

Expression and purification of HBcAg-Lys in large scale was performed as follows. An overnight culture was prepared by inoculating a single colony in 100 ml LB, 100 μg/ml Ampicillin and growing the culture overnight at 37° C. 25 ml of the preculture were diluted in 800 ml LB Ampicillin medium the next day, and the culture gorwn to an optical density OD⁶⁰⁰ of 0.6-0.8. The culture was then induced with 1 mM IPTG, and left to grow for another 4 hours. The cells were harvested and lysed essentially as described above.

HBcAg-Lys was then purified by first precipitating the protein with ammonium sulphate (30% saturation) from the cleared cell lysate, then loading the resolubilized pellet on a gel filtration column (Sephacryl S-400, Pharmacia). The pooled fractions were precipitated again with ammonium sulphate, the pellet resolubilized and loaded a second time on the same gel filtration column. The fractions were finally pooled and concentrated, and the concentration assessed using a Bradford test (BioRad).

### EXAMPLE 4

### Construction of a HBcAg Devoid of Free Cysteine Residues and Containing an Inserted Lysine Residue

A Hepatitis core Antigen (HBcAg), referred to herein as HBcAg-lys-2cys-Mut, devoid of cysteine residues at positions corresponding to 48 and 107 in SEQ ID NO:77 and containing an inserted lysine residue was constructed using the following methods.

The two mutations were introduced by first separately amplifying three fragments of the HBcAg-Lys gene prepared as described above in Example 2 with the following PCR primer combinations. PCR methods and conventional cloning techniques were used to prepare the HBcAg-lys-2cys-Mut gene.

In brief, the following primers were used to prepare fragment 1:

Primer 1: EcoRIHBcAg(s)

(SEQ ID NO: 146)

CCGGAATTCATGGACATTGACCCTTATAAAG
Primer 2: 48as

(SEQ ID NO: 150)

GTGCAGTATGGTGAGGTGAGGAATGCTCAGGAGACTC

The following primers were used to prepare fragment 2:

Primer 3: 48s

(SEQ ID NO: 151)

GSGTCTCCTGAGCATTCCTCACCTCACCATACTGCAC
Primer 4: 107as

(SEQ ID NO: 152)

CTTCCAAAAGTGAGGGAAGAAATGTGAAACCAC

The following primers were used to prepare fragment 3:

Primer 5: HBcAg149hind-as

(SEQ ID NO: 149)

CGCGTCCCAAGCTTCTAAACAACAGTAGTCTCCGGAAGCGTTGATAG
Primer 6: 107s

(SEQ ID NO: 153)

GTGGTTTCACATTTCTTCCCTCACTTTTGGAAG

Fragments 1 and 2 were then combined with PCR primers EcoRIHBcAg(s) and 107 as to give fragment 4. Fragment 4 and fragment 3 were then combined with primers EcoRIHBcAg(s) and HBcAg149hind-as to produce the full length gene. The full length gene was then digested with the EcoRI (GAATTC) and HindIII (AAGCTT) enzymes and cloned into the pKK vector (Pharmacia) cut at the same restriction sites. Expression and purification of HBcAg-lys-2cys-Mut were performed as set out in Example 3.

### EXAMPLE 5

### Construction of HBcAg1-185-Lys.

Hepatitis core Antigen (HBcAg) 1-185 was modified as described in Example 2. A part of the c/e1 epitope (residues 72 to 88) region (Proline 79 and Alanine 80) was genetically replaced by the peptide Gly-Gly-Lys-Gly-Gly (HBcAg1-185-Lys construct). The introduced Lysine residue contains a reactive amino group in its side chain that can be used for intermolecular chemical crosslinking of HBcAg particles with any antigen containing a free cysteine group. PCR methods and conventional cloning techniques were used to prepare the HBcAg1-185-Lys gene.

The Gly-Gly-Lys-Gly-Gly sequence was inserted by amplifying two separate fragments of the HBcAg gene from pEco63, as described above in Example 2 and subsequently fusing the two fragments by PCR to assemble the full length gene. The following PCR primer combinations were used:

fragment 1:

Primer 1: EcoRIHBcAg(s) (see Example 2)

Primer 2: Lys-HBcAg(as) (see Example 2)

fragment 2:

Primer 3: Lys-HBcAg(s) (see Example 2)

Primer 4: HBcAgwtHindIIII

CGCGTCCCAAGCTTCTAACATTGAGATTCCCGAGATTG (SEQ ID NO: 154)

Assembly:

Primer 1: EcoRIHBcAg(s) (see example 2)

Primer 2: HBcAgwtHindIII

The assembled full length gene was then digested with the EcoRI (GAATTC) and HindIII (AAGCTT) enzymes and cloned into the pKK vector (Pharmacia) cut at the same restriction sites.

### EXAMPLE 6

### Fusion of a Peptide Epitope in the MIR Region of HbcAg

The residues 79 and 80 of HBcAg1-185 were substituted with the epitope CεH3 of sequence VNLTWSRASG (SEQ ID NO:155). The CεH3 sequence stems from the sequence of the third constant domain of the heavy chain of human IgE. The epitope was inserted in the HBcAg1-185 sequence using an assembly PCR method. In the first PCR step, the HBcAg1-185 gene originating from ATCC clone pEco63 and amplified with primers HBcAg-wt EcoRI fwd and HBcAg-wt Hind III rev was used as template in two separate reactions to amplify two fragments containing sequence elements coding for the CεH3 sequence. These two fragments were then assembled in a second PCR step, in an assembly PCR reaction.

Primer combinations in the first PCR step: CεH3fwd with HBcAg-wt Hind III rev, and HBcAg-wt EcoRI fwd with CεH3rev. In the assembly PCR reaction, the two fragments isolated in the first PCR step were first assembled during 3 PCR cycles without outer primers, which were added afterwards to the reaction mixture for the next 25 cycles. Outer primers: HBcAg-wt EcoRI fwd and HBcAg-wt Hind III rev.

The PCR product was cloned in the pKK223.3 using the EcoRI and HindIII sites, for expression in E. coli (see Example 2). The chimeric VLP was expressed in E. coli and purified as described in Example 2. The elution volume at which the HBcAg1-185-CεH3 eluted from the gel filtration showed assembly of the fusion proteins to a chimeric VLP.

Primer Sequences:

CεH3fwd:

5′ GTT AAC TTG ACC TGG TCT CGT GCT TCT GGT GCA TCC AGG GAT CTA GTA GTC 3′
V N L T W S R A S G A80 S R D L V V86

(nucleotide sequence SEQ ID NO: 156; amino acid sequence SEQ ID NO: 157)

CεH3rev:

5′ ACC AGA AGC ACG AGA CCA GGT CAA GTT AAC ATC TTC CAA ATT ATT ACC CAC 3′
D78 E L N N G V72

(nucleotide sequence SEQ ID NO: 158; amino acid sequence SEQ ID NO: 159)

HBcAg-wt EcoRI fwd:

5′CCGgaattcATGGACATTGACCCTTATAAAG (SEQ ID NO: 146) HBcAg-wt Hind III rev:

5′CGCGTCCCaagcttCTAACATTGAGATTCCCGAGATTG (SEQ ID NO: 154)

### EXAMPLE 7

### Fusion of a Prion Peptide Epitope in the MIR Region of HbcAg

The residues 79 and 80 of HBcAg1-185 are substituted with the prion peptide epitope “murine prpshort” of sequence: GNDWEDRYYRENMYR (SEQ ID NO: 160). Two overlapping primers are designed using the same strategy described in Example 6, and the fusion protein constructed by assembly PCR. The PCR product is cloned in the pKK223.3 vector, and expressed in E. coli K802. The chimeric VLPs are expressed and purified as described in Example 3.

### EXAMPLE 8

### Fusion of a Prion Peptide Epitope to the C-Terminus of the Qβ A1 Protein Truncated at Position 19 of the CP Extension

A forward primer annealing to the sequence complementary to the 5′ end of the Qβ A1 gene and containing a suitable restriction site, and a reverse primer annealing to the 3′ end of the A1 gene, comprising a restriction site suitable for cloning, and comprising additionally a sequence element complementary to the sequence coding for the murine prion peptide prpshort, of sequence GNDWEDRYYRENMYR (SEQ ID NO: 160), are used in a PCR reaction with pQβ10 as template. The PCR product is cloned in pQβ10 (Kozlovska T. M. et al., Gene 137: 133-37 (1993)). The resulting construct encodes the A1 gene fused in frame to a sequence encoding the prpshort peptide. The chimeric VLP is expressed and purified as described in Example 1.

### EXAMPLE 9

### Insertion of a Prion Peptide Epitope Between Positions 2 and 3 of fr Coat Protein

Complementary primers coding for the sequence of the prpshort murine prion peptide of sequence GNDWEDRYYRENMYR (SEQ ID NO:160), and containing Bsp119I compatible ends and additional nucleotides enabling in frame insertion, are inserted in the Bsp119I site of the pFrd8 vector (Pushko, P. et al., Prot. Eng. 6: 883-91 (1993)) by standard molecular biology techniques. Alternatively, the overhangs of the pFrd8 vector are filled in with Klenow after digestion with Bsp119I, and oligonucleotides coding for the sequence of the prpshort murine prion peptide and additional nucleotides for in frame cloning are ligated in pFrd8 after the Klenow treatment. Clones with the insert in the right orientation are analysed by sequencing. Expression and purification of the chimeric fusion protein in E. coli JM109 or E. coli K802 is performed as described in Pushko, P. et al, Prot. Eng. 6:883-91 (1993), but for the chromatography steps which are performed using a Sepharose CL-4B or Sephacryl S-400 (Pharmacia). The cell lysate is precipitated with ammonium sulphate, and purified by two successive gel filtration purification steps, similarly to the procedure described for Qβ in Example 1.

### EXAMPLE 10

### Insertion of a Prion Peptide Epitope Between Positions 67 and 68 of Ty1 Protein p1 in the Vector pOGS8111.

Two complementary oligonucleotides coding for the murine prion peptide prpshort, of sequence GNDWEDRYYRENMYR (SEQ ID NO:160), with ends compatible with the NheI site of pOGS8111 are synthesized. Additional nucleotides are added to allow for in frame insertion of a sequence coding for the murine prpshort epitope according to the description of EP06777111. The amino acids AS and SS flanking the inserted epitope are encoded by the altered NheI sites resulting from the insertion of the oligonucleotide in the TyA(d) gene of pOGS8111.

POGS8111 is transformed into S. cervisiae strain MC2, for expression of the chimeric Ty VLP as described in EP0677111 and references therein. The chimeric Ty VLP is purified by sucrose gradient ultracentrifugation as described in EP0677111.

### EXAMPLE 11

### Insertion of a Prion Peptide Epitope in to the Major Capsid Protein L1 of Papillomavirus Type 1 (BPV-1)

A sequence coding for the murine prpshort epitope having the sequence GNDWEDRYYRENMYR (SEQ ID NO:160) is substituted to the sequence coding for amino acids 130-136 of the BPV-1 L1 gene cloned in the pFastBac1 (GIBCO/BRL) vector as described (Chackerian, B. et al., Proc. Natl. Acad. USA 96: 2373-2378 (1999)). The sequence of the construct is verified by nucleotide sequence analysis. Recombinant baculovirus is generated using the GIBCO/BRL baculovirus system as described by the manufacturer. The chimeric VLPs are purified from baculovirus infected Sf9 cells as described by Kirnbauer, R. et al., Proc. Natl. Acad. Sci. 89:12180-84 (1992) and Greenstone, H. L., et al., Proc. Natl. Acad. Sci. 95:1800-05 (1998).

### EXAMPLE 12

### Immunization of Mice With PrP-Peptides Fused to VLPs

Chimeric VLPs displaying the murine prpshort epitope of sequence GNDWEDRYYRENMYR (SEQ ID NO:160) generated in Examples 7-11 are used for immunization of mice as described in Example 15. The sera obtained from the immunized mice are analysed in a Prion protein-specific ELISA as described in Example 15.

The protective effect of the vaccine is examined by immunizing a large group of mice prior to intraperitoneal prion inoculation. Spleens, where PrP^{Sc} is detectable as early as 35 days post inoculation (35 dpi) are investigated for the presence of prions, to assess the protective effect of the vaccine in vivo. In addition, infectivity of the mentioned organs is investigated by utilizing a bioassay, as described by M. Fischer and colleagues, EMBO J. 15:1255-1264 (1996). The onset of prion disease (approximately 240 days post inoculation of wild-type mice) in immunized vs. control mice is assessed as a further read-out to confirm the efficacy of the vaccine in protecting mice against prion disease. The protective effect of the antibodies induced by the immunization procedure is further assessed in an experiment where the capability of rescuing chronically prion-infected neuroblastoma cells (ScN2a) according to a protocol published by M. Enari et al. (PNAS 98, 9295-9299, (2001)) is measured.

### EXAMPLE 13

### Introduction of Amino Acid Linker Containing a Cysteine Residue, Expression and Purification of a Truncated Form of the Mouse Prion Protein

A truncated form (aa 121-230) of the mouse prion protein (termed mPrPt) was recombinantly expressed with a GGGGCG amino acid linker (SEQ ID NO: 107) fused at its C-terminus for coupling to VLPs and Pili. The protein was fused to the N-terminus of a human Fc-fragment for purification. An enterokinase (EK) cleavage-site was introduced behind the (SEQ ID NO: 107) GGGGCG amino acid linker to cleave the Fc-part of the fusion protein after purification.

Construction of mPrPt-EK-Fc*

Mouse PrPt was amplified by PCR with the primer 5′PrP-BamHI and 3′PrP-NheI using the plasmid pBPCMVPrP-Fc as a template. pBPCMVPrP-Fc contained the wild-type sequence of the mouse prion protein. 5′PrP-BamHI had an internal BamHI site and contained an ATG and 3′PrP-NheI had an internal NheI site.

For the PCR reaction, 0.5 μg of each primer and 200 ng of the template DNA was used in the 50 μl reaction mixture (1 unit of PFX Platinum polymerase, 0.3 mM dNTPs and 2 mM MgSO4). The temperature cycles were as follows: 94° C. for 2 minutes, followed by 5 cycles of 94° C. (15 seconds), 50° C. (30 seconds), 68° C. (45 seconds), followed by 20 cycles of 94° C. (15 seconds), 64° C. (30 seconds), 68° C. (45 seconds) and followed by 68° C. for 10 minutes.

The PCR product was digested with BamHI and NheI and inserted into pCEP-SP-EK-Fc* (WO/02056905, filed by the present assignee) containing the GGGGCG linker sequence (SEQ ID NO:107) at the 5′end of the EK cleavage sequence. The resulting plasmid was named pCEP-SP-mPrPt-EK-Fc*.

All other steps were performed by standard molecular biology protocols.

Oligos:

Primer 5′ PrP-BamHI

(SEQ ID NO: 161)

5′-CGG GAT CCC ACC ATG GTG GGG GGC CTT GG-3′
Primer 3′ PrP-NheI

(SEQ ID NO: 162)

5′-CTA GCT AGC CTG GAT CTT CTC CCG-3′

Expression and Purification of mPrPt-EK-Fc*

Plasmid pCEP-SP-mPrPt-EK-Fc* was transfected into 293-EBNA cells (Invitrogen) and purified on a Protein A-sepharose column.

The protein sequence of the mPrPt-EK-Fc* is identified in SEQ ID NO:92. mPrPt after cleavage has the sequence as identified in SEQ ID NO:93 with the GGGGCG linker (SEQ ID NO:107) at its C-terminus.

The purified fusion protein mPrPt-EK-Fc* was cleaved with enterokinase and analysed on a 16% SDS-PAGE gel under reducing conditions before and after enterokinase cleavage. The gel was stained with Coomassie Brilliant Blue. The result is shown in FIG. 1. Molecular weights of marker proteins are given on the left margin of the gel in the figure. The mPrPt-EK-Fc* fusion protein could be detected as a 50 kDa band. The cleaved mPrPt protein containing the GGGGCG amino acid linker (SEQ ID NO: 107) fused to its C-terminus could be detected as a broad band between 18 and 25 kDa. The identity of mPrPt was confirmed by western blotting (data not shown). Thus, mPrPt with a C-terminal amino acid linker containing a cysteine residue, could be expressed and purified to be used for coupling to VLPs and Pili.

The samples loaded on the gel of FIG. 1 were the following.

Lane 1: Molecular weight marker. Lane 2: mPrPt-EK-Fc* before cleavage. Lane 3: mPrPt after cleavage.

Coupling of mPrPₜ to Qβ Capsid

A solution of 120 μM Qβ capsid in 20 mM Hepes, 150 mM NaCl pH 7.2 is reacted for 30 minutes with a 25 fold molar excess of SMPH (Pierce), diluted from a stock solution in DMSO, at 25° C. on a rocking shaker. The reaction solution is subsequently dialyzed twice for 2 hours against 1 L of 20 mM Hepes, 150 mM NaCl, pH 7.2 at 4° C. The dialyzed Qβ reaction mixture is then reacted with the mPrP, solution (end concentrations: 60 μM Qβ, 60 μM mPrPt) for four hours at 25° C. on a rocking shaker. Coupling products are analysed by SDS-PAGE.

Coupling of mPrPₜ to fr Capsid Protein

A solution of 120 μM fr capsid protein in 20 mM Hepes, 150 mM NaCl pH 7.2 is reacted for 30 minutes with a 25 fold molar excess of SMPH (Pierce), diluted from a stock solution in DMSO, at 25° C. on a rocking shaker. The reaction solution is subsequently dialyzed twice for 2 hours against 1 L of 20 mM Hepes, 150 mM NaCl, pH 7.2 at 4° C. The dialyzed fr reaction mixture is then reacted with the mPrPₜ solution (end concentrations: 60 μM fr, 60 μM mPrPₜ) for four hours at 25° C. on a rocking shaker. Coupling products are analysed by SDS-PAGE.

Coupling of mPrPt to HBcAg-Lys-2cys-Mut

A solution of 120 μM HBcAg-Lys-2cys-Mut capsid in 20 mM Hepes, 150 mM NaCl pH 7.2 is reacted for 30 minutes with a 25 fold molar excess of SMPH (Pierce), diluted from a stock solution in DMSO, at 25° C. on a rocking shaker. The reaction solution is subsequently dialyzed twice for 2 hours against 1 L of 20 mM Hepes, 150 mM NaCl, pH 7.2 at 4° C. The dialyzed 1HBcAg-Lys-2cys-Mut reaction mixture is then reacted with the mPrPₜ solution (end concentrations: 60 μM HBcAg-Lys-2cys-Mut, 60 μM mPrPₜ) for four hours at 25° C. on a rocking shaker. Coupling products are analysed by SDS-PAGE.

Coupling of mPrPₜ to Pili

A solution of 125 μM Type-1 pili of E. coli in 20 mM Hepes, pH 7.4, is reacted for 60 minutes with a 50-fold molar excess of cross-linker SMPH (Pierce), diluted from a stock solution in DMSO, at RT on a rocking shaker. The reaction mixture is desalted on a PD-10 column (Amersham-Pharmacia Biotech). The protein-containing fractions eluating from the column are pooled, and the desalted derivatized pili protein is reacted with the mPrPₜ solution (end concentrations: 60 μM pili, 60 μM mPrPₜ) for four hours at 25° C. on a rocking shaker. Coupling products are analysed by SDS-PAGE.

### EXAMPLE 14

### Coupling of Prion Peptides to Qβ Capsid Protein: Prion Peptide Vaccines

The following prion peptides were chemically synthesized: CSAMSRPMIFGNDWEDRYYRENMYR (“murine cprplong”)(SEQ ID NO: 163) and CGNDWEDRYYRENMYR (“murine cprpshort”) (SEQ ID NO: 164), which comprise an added N-terminal cysteine residue for coupling to VLPs and Pili, and used for chemical coupling to Qβ as described in the following.

A solution of 5 ml of 140 μM Qβ capsid protein in 20 mM Hepes, 150 mM NaCl pH 7.4 was reacted for 30 minutes with 108 μl of a 65 mM solution of SMPH (Pierce) in H₂O at 25° C. on a rocking shaker. The reaction solution was subsequently dialyzed twice for 2 hours against 5 L of 20 mM Hepes, 150 mM NaCl, pH 7.4 at 4° C. 100 μl of the dialyzed reaction mixture was then reacted either with 1.35 μl of a 2 mM stock solution (in DMSO) of the peptide cprpshort (1:2 peptide/Qβ capsid protein ratio) or with 2.7 μl of the same stock solution (1:1 peptide/Qβ ratio). 1 μl of a 10 mM stock solution (in DMSO) of the peptide cprplong was reacted with 100 μl of the dialyzed reaction mixture. The coupling reactions were performed over night at 15° C. in a water bath. The reaction mixtures were subsequently dialyzed 24 h against 2×5 L of 20 mM Hepes, 150 mM NaCl, pH 7.4 at 4° C.

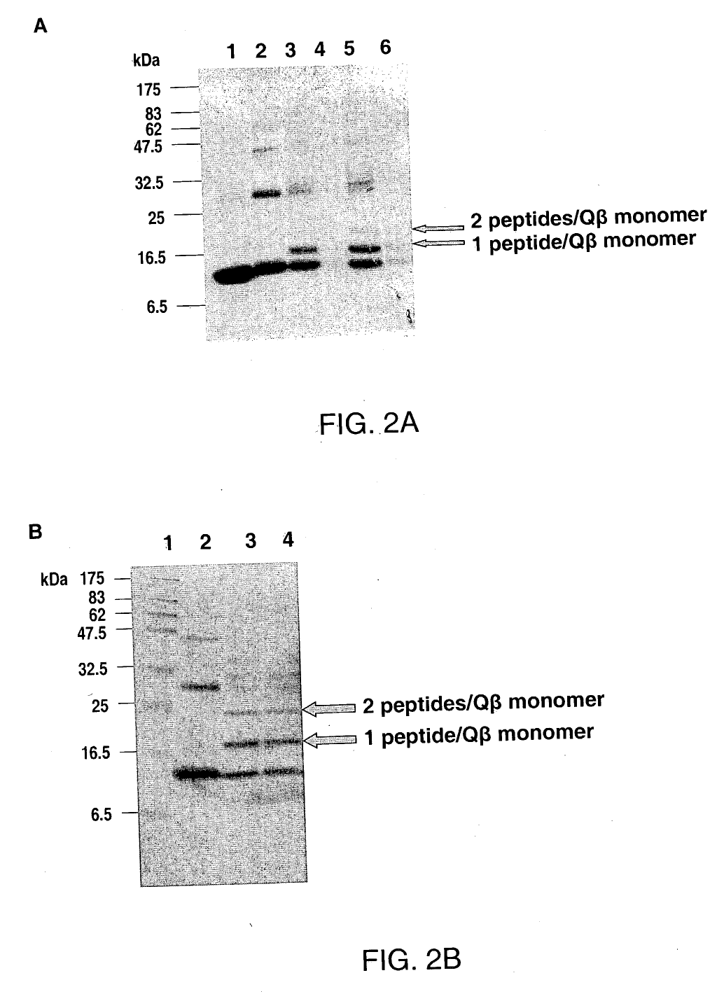

The coupled products were centrifuged and supernatants and pellets were analysed on 16% SDS-PAGE gels under reducing conditions. Gels were stained with Coomassie Brilliant Blue. The results are shown in FIG. 2. Molecular weights of marker proteins are given on the left margin of the gel in the figure. The bands at a molecular weight between 16.5 and 25 kDa clearly demonstrated the covalent coupling of the peptides cprpshort and cprplong to Qβ capsid protein.

The samples loaded on the gel of FIG. 2A were the following:

Lane 1: purified Qβ capsid protein. Lane 2: derivatized Qβ capsid protein before coupling. Lanes 3-6: Qβ capsid protein-cprpshort couplings with a 1:2 peptide/Qβ ratio (lanes 3 and 4) and 1:1 peptide/Qβ ratio (lanes 5 and 6). Soluble fractions (lanes 3 and 5) and insoluble fractions (lanes 4 and 6) are shown.

The samples loaded on the gel of FIG. 2B were the following:

Lane 1: Molecular weight marker. Lane 2: derivatized Qβ capsid protein before coupling. Lane 3 and 4: Qβ ▴capsid protein-cprplong coupling reactions. Soluble fraction (lane 3) and insoluble fraction (lane 4) are shown.

Coupling of Prion Peptides to fr Capsid Protein

A solution of 120 μM fr capsid protein in 20 mM Hepes, 150 mM NaCl pH 7.2 is reacted for 30 minutes with a 10 fold molar excess of SMPH (Pierce)), diluted from a stock solution in DMSO, at 25° C. on a rocking shaker. The reaction solution is subsequently dialyzed twice for 2 hours against 1 L of 20 mM Hepes, 150 mM NaCl, pH 7.2 at 4° C. The dialyzed fr reaction mixture is then reacted with equimolar concentration of peptide cprpshort or a ration of 1:2 cprplong/fr over night at 16° C. on a rocking shaker. Coupling products are analysed by SDS-PAGE.

Coupling of Prion Peptides to HBcAg-Lys-2cys-Mut

A solution of 120 μM HBcAg-Lys-2cys-Mut in 20 mM Hepes, 150 mM NaCl pH 7.2 is reacted for 30 minutes with a 10 fold molar excess of SMPH (Pierce), diluted from a stock solution in DMSO, at 25° C. on a rocking shaker. The reaction solution is subsequently dialyzed twice for 2 hours against 1 L of 20 mM Hepes, 150 mM NaCl, pH 7.2 at 4° C. The dialyzed HBcAg-Lys-2cys-Mut reaction mixture is then reacted with equimolar concentration of peptide cprpshort or a ration of 1:2 cprplong/HBcAg-Lys-2cys-Mut over night at 16° C. on a rocking shaker. Coupling products are analysed by SDS-PAGE.

Coupling of Prion Peptides to Pili

A solution of 125 μM Type-1 pili of E. coli in 20 mM Hepes, pH 7.4, is reacted for 60 minutes with a 50-fold molar excess of cross-linker SMPH (Pierce), diluted from a stock solution in DMSO, at RT on a rocking shaker. The reaction mixture is desalted on a PD-10 column (Amersham-Pharmacia Biotech). The protein-containing fractions eluating from the column are pooled, and the desalted derivatized pili protein is reacted with the prion peptides in equimolar or in a ratio of 1:2 peptide pili over night at 16° C. on a rocking shaker. Coupling products are analysed by SDS-PAGE.

### EXAMPLE 15

### Immunization of Mice With VLP-PrP Peptide Conjugates

Immunization of Mice With PrP-Peptides Coupled to Qβ

Female C57BL/6 mice were immunized with the murine prion peptide cprpshort coupled to Qβ (in the example termed “mPrPs-Qβ”) or the peptide cprplong coupled to Qβ (in the example termed “mPrP1-Qβ”), administered in alum, as CFA/IFA-emulsion, or with no adjuvant. The murine prion peptides cprpshort, cprplong (in the example just referred to cprpshort or cprplong) or Qβ served as negative controls. In addition, female Prnpo/o mice (H. R. Büeler et al., Cell 1993) were injected with mPrPs-Qβ or mPrPl-Qβ. 50 μg of total protein of mPrPs-Qβ, mPrP1-Qβ or Qβ and 100 μg of cprpshort and cprplong were diluted in PBS to a final volume of 200 μl and administered subcutaneously on day 0, day 14, and day 28. In addition, 50 μg mPrPs-Qβ or mPrPl-Qβ were applied intraperitoneally in 500 μl (1 mg) alum or subcutaneously as CFA-emulsion (100 μl) on day 0 or intraperitoneally as IFA-emulsion (100 μl) on day 14 and day 28. Mice were bled retroorbitally four and thirteen days after immunization. Serum was analyzed using a prion protein-specific ELISA.

Prion Protein-Specific ELISA

Prion protein-specific ELISA (enzyme-linked immunosorbent assay) described by Heppner et al. (Science 294:178-182 (2001)) was used to analyse serum of mice immunized with mPrPs-Qβ, mPrP1-Qβ and controls. In detail, 384-well plates were coated with 5 μg/ml recombinant mouse PrP23-231 (Hornemann, S., et al., FEBS Lett. 413:277-281 (1997)) or PrP121-230 (Hornemann, S. & Glockshuber R., J Mol Biol. 261:614-619 (1996)) in PBS overnight at 4° C. After three washing steps with PBS, 0.1% Tween, the plates were blocked for two hours with 5% bovine serum albumin (BSA), PBS, 0.1% Tween at room temperature. After washing, the plates were incubated with 30 μl of two- or threefold serially diluted mouse serum in 1% BSA, PBS, 0.1% Tween in duplicates for two hours at room temperature. After four washing steps, the plates were probed with horse-radish peroxidase (HRP)-conjugated goat anti-mouse IgG+A+M (H+L, 1:1000 dilution), HRP-conjugated rat anti-mouse IgM (1:1000), HRP-conjugated rabbit anti-mouse IgG (1:1000), HRP-conjugated rabbit anti-mouse IgG1 (1:1000), HRP-conjugated rabbit anti-mouse IgG2a (1:1000), and HRP-conjugated rabbit anti-mouse IgG2b (1:1000) from Zymed Laboratories, San Francisco, Calif. Plates were developed with 2.2°-azino-di-ethyl-benzthiazolinsulfonate (ABTS) in 0.1 M NaH2PO4, pH 4 and H₂O₂. Optical density (OD) was measured at 405 nm. Endpoint ELISA titers were determined by counting the number of titrations possible above two times the average of the background. Omission of serum and recombinant PrP served as background and corresponded to preimmune serum values. Serum of 6H4μ-transgenic mice (Heppner, F. L., et al., Science 294:178-182 (2001)) and Prnpo/o mice immunized with mPrPs-Qβ or mPrP1-Qβ were used as positive controls, preimmune serum as negative control.

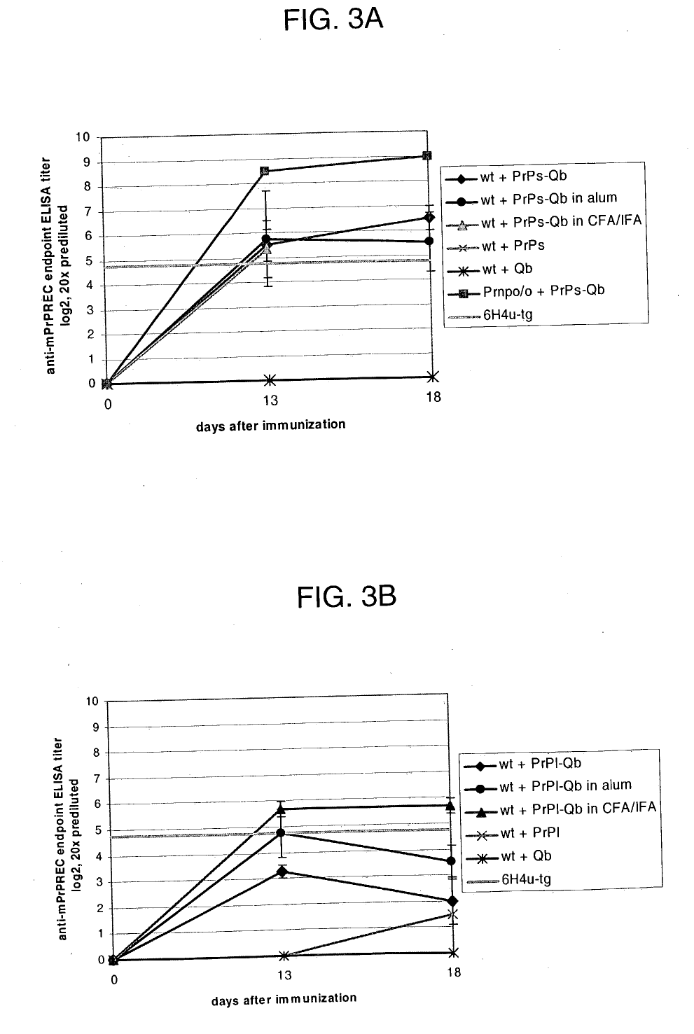

Immunization with mPrPs-Qβ and mPrP1-Qβ resulted in antibodies recognizing full-length recombinant prion protein. FIG. 3 shows total immunoglobulins (IgG+A+M) detected on day 13 and day 4 after the first boost. mPrPs-Qβ raised a higher anti-PrP titer than mPrP1-Qβ. Compared to 6H4μ-transgenic mice, anti-PrP serum antibody levels induced by mPrPs-Qβ were higher, anti-PrP serum antibody levels induced by mPrP1-Qβ were lower. Administration of mPrPs-Qβ in alum or CFA/IFA did not increase the anti-PrP titer. In contrast, injection of mPrP1-Qβ in alum or CFA/IFA seemed to increase the anti-PrP titer. Further, in one of four mice immunized with mPrPs-Qβ or mPrP1-Qβ emulsified in CFA/IFA an anti-PrP titer could not be detected. Based on these observations, mice were immunized with mPrPs-Qβ without adjuvant in future experiments.

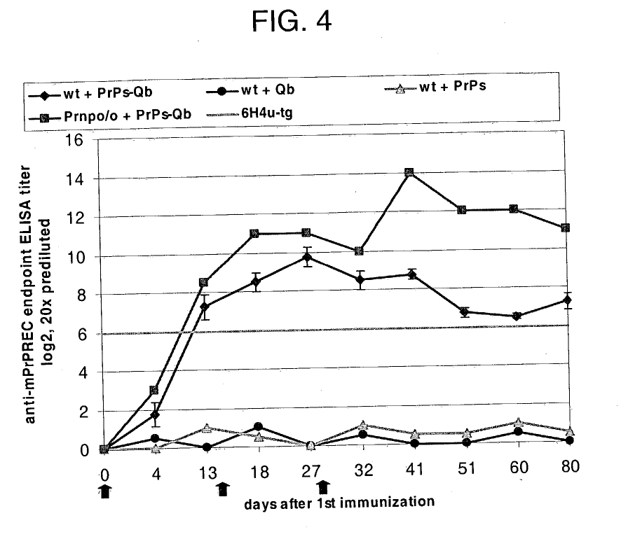

The long-term anti-PrP antibody response of mPrPs-Qβ is shown in FIG. 4 (ELISA of total immunoglobulins). On day 13, an anti-PrP titer was detected in wild-type and Prnpo/o mice. Moreover, three immunizations were sufficient to induce an anti-PrP antibody response lasting two and a half months. In addition, immunoglobulin isotypes were analysed on day 13 after the second boost resulting mainly in anti-PrP antibodies of the IgG2a class.

### EXAMPLE 16

### Detection of PrP^{Sc} in Spleens by Western Blot-Analysis

A large group of female C57BL/6 mice were immunized with mPrPs-Qβ, PrPs-peptide, Qβ or received no treatment following the immunization protocol described in Example 15. At 25 days after first immunization, the mice were inoculated intraperitoneally with scrapie prions (RML strain, passage 5) (Büeler, H. R., et al., Cell 73:1339-1347 (1993)). Spleens were harvested at 62 days after inoculation (dpi) and accumulation of PrP^{Sc} was analysed. 10% (w/v) homogenates of spleen were prepared as described (Büeler, H. R., et al., Cell 73:1339-1347 (1993)). Sodium phosphotungstic acid (NaPTA) precipitation was performed according to Safar J., et al., Nat Med 4:1157-1165 (1998). 0.5 mg total protein from spleen homogenates was treated with proteinase K (20 μg/ml, 30 min, 37° C.) before precipitation. NaPTA-pellet samples were electrophoresed through a 16% SDS-polyacrylamide gel. Proteins were transferred to nitrocellulose by wet blotting. Membranes were blocked with 5% TopBock (Juro/Merck, Switzerland) in Tris-buffered saline-Tween (TBS-T) and incubated with monoclonal anti-PrP antibody ICSM18 (1:20000). HRP-conjugated rabbit anti-mouse IgG₁ (1:10000, Zymed) was used as secondary antibody. Bands were detected by enhanced chemiluminescence (Pierce) and quantified using the ID image analysis software (Kodak).

### EXAMPLE 17

### Protection Against Prion Disease

Immunization of wild-type and Prnpo/o mice with mPrPs-Qβ and mPrP1-Qβ resulted in antibodies recognizing recombinant full-length PrP, although slightly higher anti-PrP titers were detected in mice devoid of PrP when compared to wild-type mice. This difference might be attributable to B cell tolerance against the self-protein PrP^{C}.

Notably, three administrations of mPrPs-Qβ were sufficient to induce a long-lasting anti-PrP antibody response in wild-type and Prnpo/o mice outreaching the anti-PrP titer of 6H41μ-transgenic mice.

To determine whether anti-PrP antibodies induced by mPrPs-Qβ protect against prion disease, a large group of mice has been immunized prior to intraperitoneal prion inoculation. In order to test a putative inhibition of prions in vivo, spleens are, then, investigated for the presence of prions, where PrP^{Sc} is detectable as early as 35 days post inoculation (35 dpi). In addition, infectivity of the mentioned organs is investigated by utilizing a bioassay, as described by M. Fischer and colleagues, EMBO J. 15:1255-1264 (1996). Finally, the onset of prion disease (approximately 240 days post inoculation of wild-type mice) in immunized vs. control mice is assessed in order to demonstrate an altered susceptibility of immunized mice. At the same time, serum of immunized mice is tested for the capability of rescuing chronically prion-infected neuroblastoma cells (ScN2a) according to a protocol published by Enari, M., et al., PNAS 98:9295-9299 (2001)). First results indicate reduced accumulation of PrP^{Sc} in mice vaccinated with mPrPs-Qβ.

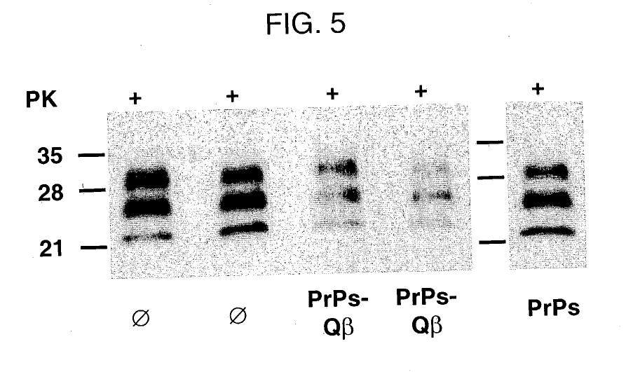

PrP^{Sc} accumulation was detected in scrapie-infected mice that had received no treatment, mPrPs-Qβ or PrPs-peptide. However, the level of deposition of PrP^{Sc} was markedly reduced in mice immunized with mPrPs-Qβ (FIG. 5). A two- to fourfold reduction was observed upon quantification, possibly indicating delayed progression of pathogenesis.

Further, since PrP^{C} is expressed in most tissues of the body, putative autoimmune phenomena are investigated in immunized mice, e.g. by analyzing deposition of autoimmune complexes in kidney and skin as well as in organs with high PrP levels, such as CNS.

### EXAMPLE 18

### Expression and Purification of Recombinant AP205 VLP

A. Expression of Recombinant AP205 VLP

E. coli JM109 was transformed with plasmid pAP283-58. 5 ml of LB liquid medium with 20 μg/ml ampicillin were inoculated with a single colony, and incubated at 37° C. for 16-24 h without shaking.

The prepared inoculum was diluted 1:100 in 100-300 ml of LB medium, containing 20 μg/ml ampicillin and incubated at 37° C. overnight without shaking. The resulting second inoculum was diluted 1:50 in 2TY medium, containing 0.2% glucose and phosphate for buffering, and incubated at 37° C. overnight on a shaker. Cells were harvested by centrifugation and frozen at −80° C.

B. Purification of Recombinant AP205 VLP

Solutions and Buffers:

1. Lysis Buffer

50 mM Tris-HCl pH 8.0 with 5 mM EDTA, 0.1% tritonX100 and PMSF at 5 micrograms per ml.

2. SAS

Saturated ammonium sulphate in water

3. Buffer NET.

20 mM Tris-HCl, pH 7.8 with 5 mM EDTA and

150 mM NaCl.

4. PEG

40% (w/v) polyethylenglycol 6000 in NET

Lysis

Frozen cells were resuspended in lysis buffer at 2 ml/g cells. The mixture was sonicated with 22 kH five times for 15 seconds, with intervals of 1 min to cool the solution on ice. The lysate was then centrifuged for 20 minutes at 12 000 rpm, using a F34-6-38 rotor (Ependorf). The centrifugation steps described below were all performed using the same rotor, except otherwise stated. The supernatant was stored at 4° C., while cell debris were washed twice with lysis buffer. After centrifugation, the supernatants of the lysate and wash fractions were pooled.

Fractionation

Ammonium-sulphate precipitation can be further used to purify AP205 VLP. In a first step, a concentration of ammonium-sulphate at which AP205 VLP does not precipitate is chosen. The resulting pellet is discarded. In the next step, an ammonium sulphate concentration at which AP205 VLP quantitatively precipitates is selected, and AP205 VLP is isolated from the pellet of this precipitation step by centrifugation (14 000 rpm, for 20 min). The obtained pellet is solubilised in NET buffer.

Chromatography

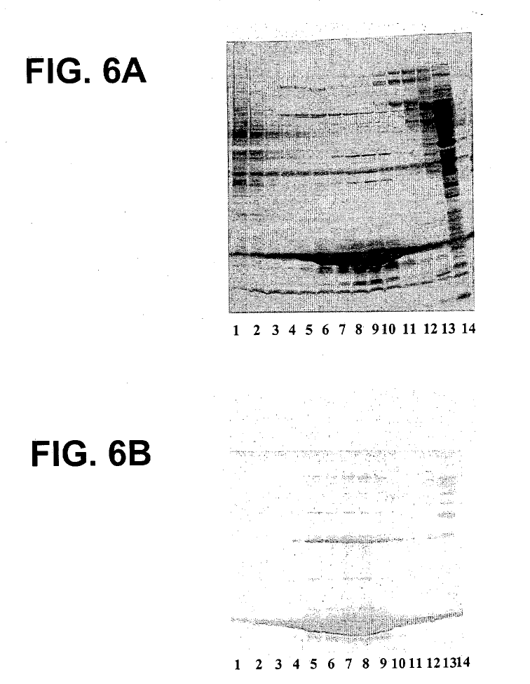

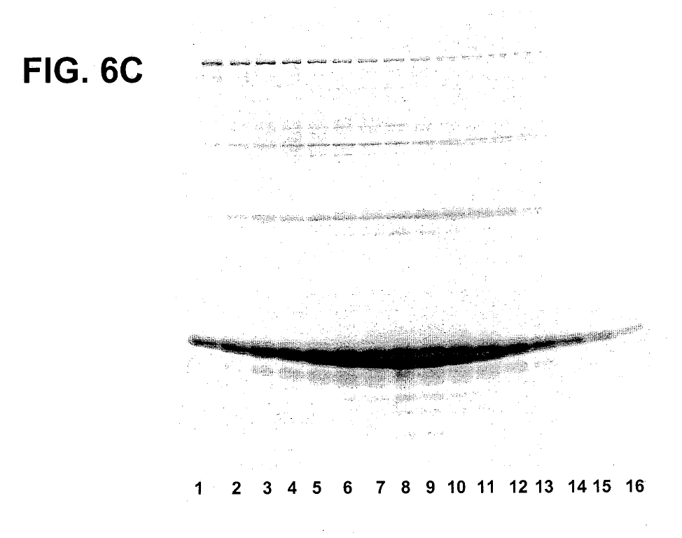

The capsid protein from the pooled supernatants was loaded on a Sepharose 4B column (2.8×70 cm), and eluted with NET buffer, at 4 ml/hour/fraction. Fractions 28-40 were collected, and precipitated with ammonium sulphate at 60% saturation. The fractions were analyzed by SDS-PAGE and Western Blot with an antiserum specific for AP205 prior to precipitation (FIG. 6A and FIG. 6B). The pellet isolated by centrifugation was resolubilized in NET buffer, and loaded on a Sepharose 2B column (2.3×65 cm), eluted at 3 ml/h/fraction. Fractions were analysed by SDS-PAGE, and fractions 44-50 were collected, pooled and precipitated with ammonium sulphate at 60% saturation. The pellet isolated by centrifugation was resolubilized in NET buffer, and purified on a Sepharose 6B column (2.5×47 cm), eluted at 3 ml/hour/fraction. The fractions were analysed by SDS-PAGE. Fractions 23-27 were collected, the salt concentration adjusted to 0.5 M, and precipitated with PEG 6000, added from a 40% stock in water and to a final concentration of 13.3%. The pellet isolated by centrifugation was resolubilized in NET buffer, and loaded on the same Sepharose 2B column as above, eluted in the same manner. Analysis of the fractions by SDS-PAGE is shown in FIG. 6C. Fractions 43-53 were collected, and precipitated with ammonium sulphate at a saturation of 60%. The pellet isolated by centrifugation was resolubilized in water, and the obtained protein solution was extensively dialyzed against water. About 10 mg of purified protein per gram of cells could be isolated.

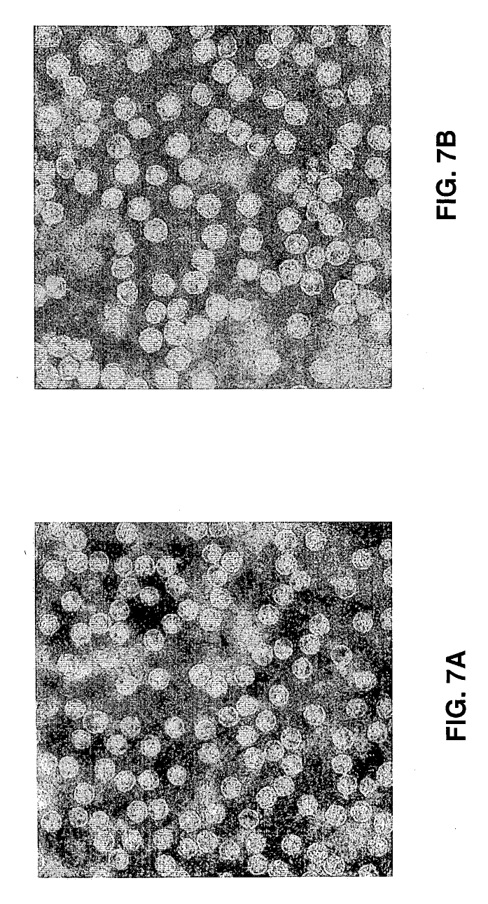

Examination of the virus-like particles in Electron microscopy showed that they were identical to the phage particles (FIGS. 7A and 7B).

FIG. 6A shows in the top panel, the silver-stained SDS-PAGE run under reducing conditions of the fractions of the first Sepharose 4B chromatography step. Lane 1-13 were loaded with every second fraction from fraction 20 to 44. Fraction 50 was loaded in lane 14. A second gel was loaded with the same fractions and analysed by Western blotting with an anti-serum specific for AP205, and is shown in the lower panel (FIG. 6B).

FIG. 6C shows the silver-stained SDS-PAGE run under reducing conditions of the fractions of the last Sepharose 2B chromatography step. Fractions 38-54 are loaded in Lane 1-16.

FIG. 7A shows an EM picture of AP205 phage particles, while an EM picture of self assembled particles of recombinant AP205 VLP is shown in FIG. 7B.

### EXAMPLE 19

### Murine Prion Protein dimer

The dimer of mouse prion protein, mPrPt (aa 121-230), is constructed by assembly PCR by amplifying first the cDNA encoding mPrPt of example 13 with two sets of primers in two individual PCR reactions, and then splicing both fragments in a “splice overlap assembly PCR”. In the first set of primer, used to obtain the first PCR fragment using mPrPt cDNA as template, the forward primer anneals to a sequence complementary to the 5′-end of mPrPt cDNA, and contains a restriction site for cloning. The reverse primer is composed of three sequence elements. The more 5′ part of the primer anneals to the 5′end of mPrPt cDNA, while the 3′ part of the primer anneals to the 3′ end of mPrPt cDNA. The central sequence element is complementary to a sequence coding for the amino acid linker GGGGS (SEQ ID NO: 101).

In the second set of primer, the forward primer contains three sequence elements as well. The more 5′ sequence element anneals to a sequence complementary to the 3′ end of mPrPt cDNA. The central sequence element anneals to a sequence complementary to a sequence coding for the amino acid linker GGGGS (SEQ ID NO: 101). The more 3′ sequence element anneals to a sequence complementary to the 5′end of mPrPt cDNA. The reverse primer anneals to the 3′ end of mPrPt cDNA, and contains a restriction site for cloning.

The cDNA of the dimer is assembled in a PCR reaction where the two PCR fragments obtained in the first PCR reactions are assembled and amplified with the forward primer of the first set of primer and the reverse primer of the second set of primer. These primers contain restriction sites suitable for cloning into either the pMod vectors described in example 4 of WO/02056905, filed by the present assignee, or the pCEP-SP-EK-Fc* vector (WO/02056905, filed by the present assignee), leading to the introduction of an amino acid linker containing a cysteine residue as second attachment site to the C-terminus of the mPrPt dimer. The protein is expressed, purified and coupled as described in example 13 or as described for proteins cloned in the pMod vectors described in example 4 of WO/02056905, filed by the present assignee.

What is claimed is:

1. A composition comprising:
(a) a virus-like particle; and
(b) at least one antigen or antigenic determinant, wherein said antigen or said antigenic determinant is a prion protein (PrP) or a dimer thereof, or a PrP peptide, and
wherein said at least one antigen or antigenic determinant is bound to said virus-like particle.

2. The composition of claim 1, wherein said virus-like particle (a) is a recombinant virus-like particle.

3. The composition of claim 1, wherein said virus-like particle comprises recombinant proteins, or fragments thereof, selected from the group consisting of:
(a) recombinant proteins of Hepatitis B virus;
(b) recombinant proteins of measles virus;
(c) recombinant proteins of Sindbis virus;
(d) recombinant proteins of Rotavirus;
(e) recombinant proteins of Foot-and-Mouth-Disease virus;
(f) recombinant proteins of Retrovirus;
(g) recombinant proteins of Norwalk virus;
(h) recombinant proteins of Alphavirus;
(i) recombinant proteins of human Papilloma virus;
(j) recombinant proteins of Polyoma virus;
(k) recombinant proteins of bacteriophages;
(l) recombinant proteins of RNA-phages;
(m) recombinant proteins of Ty;
(n) recombinant proteins of Qβ-phage;
(o) recombinant proteins of GA-phage;
(p) recombinant proteins of fr-phage; and
(q) fragments of any of the recombinant proteins from (a) to (p).

4. The composition of claim 1, wherein said virus-like particle is Hepatitis B virus core antigen.

5. The composition of claim 1, wherein said virus-like particle comprises recombinant proteins, or fragments thereof, of a RNA-phage.

6. The composition of claim 5, wherein said RNA-phage is selected from the group consisting of:
(a) bacteriophage Qβ;
(b) bacteriophage R17;
(c) bacteriophage fr;
(d) bacteriophage GA;
(e) bacteriophage SP;
(f) bacteriophage MS2;
(g) bacteriophage M11;
(h) bacteriophage MX1;
(i) bacteriophage NL95;
(k) bacteriophage f2;
(l) bacteriophage PP7; and
(m) bacteriophage AP205.

7. The composition of claim 1, wherein said virus-like particle comprises recombinant proteins, or fragments thereof, of RNA-phage Qβ.

8. The composition of claim 1, wherein said virus-like particle comprises recombinant proteins, or fragments thereof, of RNA-phage fr.

9. The composition of claim 1, wherein said virus-like particle comprises recombinant proteins, or fragments thereof, of RNA-phage AP205.

10. The composition of claim 1, wherein said at least one antigen or antigenic determinant is bound to said virus-like particle by at least one covalent bond.

11. The composition of claim 1, wherein said at least one antigen or antigenic determinant is bound to said virus-like particle by at least one covalent bond, and wherein said covalent bond is a non-peptide bond.

12. The composition of claim 1, wherein said at least one antigen or antigenic determinant is fused to said virus-like particle.

13. The composition of claim 1, wherein said antigen or antigenic determinant is a prion protein or a dimer thereof.

14. The composition of claim 1, wherein said antigen or antigenic determinant is selected from the group consisting of:
(a) human prion protein or a dimer thereof;
(b) bovine prion protein or a dimer thereof;
(c) sheep prion protein or a dimer thereof;
(d) elk prion protein or a dimer thereof;
(e) mule deer prion protein or a dimer thereof;
(f) white-tailed deer prion protein or a dimer thereof;
(g) pig prion protein or a dimer thereof;
(h) chicken prion protein or a dimer thereof;
(i) mouse prion protein or a dimer thereof;
(j) goat prion protein or a dimer thereof; and
(k) a peptide or fragment thereof of any prion protein of (a)-(j).

15. The composition of claim 1, wherein said antigen or antigenic determinant has an amino acid sequence selected from the group consisting of:
(a) the amino acid sequence of SEQ ID NO:79;
(b) the amino acid sequence of SEQ ID NO:80;
(c) the amino acid sequence of SEQ ID NO:81;
(d) the amino acid sequence of SEQ ID NO:82;
(e) the amino acid sequence of SEQ ID NO:83;
(f) the amino acid sequence of SEQ ID NO:84;
(g) the amino acid sequence of SEQ ID NO:85;
(h) the amino acid sequence of SEQ ID NO:86;
(i) the amino acid sequence of SEQ ID NO:87;
(j) the amino acid sequence of SEQ ID NO:88; and
(k) the amino acid sequence of a fragment of any of SEQ ID NOS:79-88.

16. The composition of claim 1, wherein said antigen or antigenic determinant is a PrP peptide.

17. The composition of claim 16, wherein said prion peptide is selected from the group consisting of:
(a) human prion peptide;
(b) bovine prion peptide;
(c) sheep prion peptide;
(d) elk prion peptide;
(e) mule deer prion peptide;
(f) white-tailed deer prion peptide;
(g) pig prion peptide;
(h) chicken prion peptide;
(i) mouse prion peptide;
(j) goat prion peptide; and
(k) a fragment of any prion peptide of (a)-(j).

18. The composition of claim 1, wherein said antigen or antigenic determinant is a PrP peptide comprising at least one antigenic site of a prion protein

19. The composition of claim 1, wherein said antigen or antigenic determinant is a PrP peptide comprising an amino acid sequence selected from the group consisting of:
+M1 !,1 (a)? ,3 SAMSRPIIHFGSDYEDRYYRENMHR;? ,22 (SEQ ID NO: 112)? ,32 ? ! !(b)? GSDYEDRYYRENMHR;? (SEQ ID NO: 113)? ! !(c)? SAMSRPLIHFGSDYEDRYYRENMHR;? (SEQ ID NO: 114)? ! !(d)? GSDYEDRYYRENMHR;? (SEQ ID NO: 115)? ! !(e)? SAMSRPLIHFGNDYEDRYYRENMYR;? (SEQ ID NO: 116)? ! !(f)? GNDYEDRYYRENMYR;? (SEQ ID NO: 117)? ! !(g)? SAMNRPLIHFGNDYEDRYYRENMYR;? (SEQ ID NO: 118)? ! !(h)? GNDYEDRYYRENMYR;? (SEQ ID NO: 119)? ! !(i)? SAMSRPLIHFGNDYEDRYYRENMYR; and? (SEQ ID NO: 120)? ! !(j)? GNDYEDRYYRENMYR.? (SEQ ID NO: 121)? !
!

20. The composition of claim 1, wherein said antigen or antigenic determinant further comprises at least one second attachment site selected from the group consisting of:
(a) an attachment site not naturally occurring with said antigen or antigenic determinant; and
(b) an attachment site naturally occurring with said antigen or antigenic determinant.

21. The composition of claim 20, wherein said antigen or antigenic determinant with said at least one second attachment site comprises an amino acid sequence selected from the group consisting of:
+M1 !,1 (a)? ,3 CSAMSRPIIHFGSDYEDRYYRENMHR;? ,22 (SEQ ID NO: 122)? ,32 ? ! !(b)? CGSDYEDRYYRENMHR;? (SEQ ID NO: 123)? ! !(c)? CSAMSRPLLHEGSDYEDRYYRENMHR;? (SEQ ID NO: 124)? ! !(d)? CGSDYEDRYYRENMHR;? (SEQ ID NO: 125)? ! !(e)? CSAMSRPLIHFGNDYEDRYYRENMYR;? (SEQ ID NO: 126)? ! !(f)? CGNDYEDRYYRENMYR;? (SEQ ID NO: 127)? ! !(g)? CSAMNRPLIHFGNDYEDRYYRENMYR;? (SEQ ID NO: 128)? ! !(h)? CGNDYEDRYYRENMYR;? (SEQ ID NO: 129)? ! !(i)? CSAMSRPLIHFGNDYEDRYYRENMYR; and? (SEQ ID NO: 130)? ! !(j)? CGNDYEDRYYRENMYR.? (SEQ ID NO: 131)? !
!

22. A composition comprising:
(a) a core particle with at least one first attachment site; and
(b) at least one antigen or antigenic determinant with at least one second attachment site,
wherein said antigen or antigenic determinant is a prion protein (PrP) or a dimer thereof, or a prion peptide, and wherein said second attachment site is selected from the group consisting of:
(i) an attachment site not naturally occurring with said antigen or antigenic determinant; and
(ii) an attachment site naturally occurring with said antigen or antigenic determinant,
wherein said second attachment site is capable of association to said first attachment site; and wherein said antigen or antigenic determinant and said core particle interact through said association to form an ordered and repetitive antigen array.

23. The composition of claim 22, wherein said second attachment site is capable of association to said first attachment site through at least one non-peptide bond.

24. The composition of claim 22, wherein said core particle is selected from the group consisting of:
(a) a virus;
(b) a virus-like particle;
(c) a bacteriophage;
(d) a bacterial pilus;
(e) a viral capsid particle; and
(f) a recombinant form of (a), (b), (c), (d) or (e).

25. The composition of claim 22, wherein said core particle is selected from the group consisting of:
(a) a virus-like particle;
(b) a bacterial pilus; and
(c) a virus-like particle of a RNA-phage.

26. The composition of claim 22, wherein said virus-like particle (a) is a recombinant virus-like particle.

27. The composition of claim 22, wherein said virus-like particle comprises recombinant proteins, or fragments thereof, selected from the group consisting of:
(a) recombinant proteins of Hepatitis B virus;
(b) recombinant proteins of measles virus;
(c) recombinant proteins of Sindbis virus;
(d) recombinant proteins of Rotavirus;
(e) recombinant proteins of Foot-and-Mouth-Disease virus;
(f) recombinant proteins of Retrovirus;
(g) recombinant proteins of Norwalk virus;
(h) recombinant proteins of Alphavirus;
(i) recombinant proteins of human Papilloma virus;
(j) recombinant proteins of Polyoma virus;
(k) recombinant proteins of bacteriophages;
(l) recombinant proteins of RNA-phages;
(m) recombinant proteins of Ty;
(n) recombinant proteins of Qβ-phage;
(o) recombinant proteins of GA-phage;
(p) recombinant proteins of fr-phage; and
(q) fragments of any of the recombinant proteins from (a) to (p).

28. The composition of claim 22, wherein said virus-like particle is Hepatitis B virus core antigen.

29. The composition of claim 22, wherein said virus-like particle comprises recombinant proteins, or fragments thereof, of a RNA-phage.

30. The composition of claim 29, wherein said RNA-phage is selected from the group consisting of:
(a) bacteriophage Qβ;
(b) bacteriophage R17;
(c) bacteriophage fr;
(d) bacteriophage GA;
(e) bacteriophage SP;
(f) bacteriophage MS2;
(g) bacteriophage M11;
(h) bacteriophage MX1;
(i) bacteriophage NL95;
(k) bacteriophage f2;
(l) bacteriophage PP7; and
(m) bacteriophage AP205.

31. The composition of claim 22, wherein said virus-like particle comprises recombinant proteins, or fragments thereof, of RNA-phage Qβ.

32. The composition of claim 22, wherein said virus-like particle comprises recombinant proteins, or fragments thereof, of RNA-phage fr.

33. The composition of claim 22, wherein said virus-like particle comprises recombinant proteins, or fragments thereof, of RNA-phage AP205

34. The composition of claim 22, wherein said second attachment site is capable of association to said first attachment site through at least one covalent bond.

35. The composition of claim 22, wherein said second attachment site is capable of association to said first attachment site through at least one covalent bond, and wherein said covalent bond is a non-peptide bond.

36. The composition of claim 22, wherein said antigen or antigenic determinant is a prion protein or a dimer thereof.

37. The composition of claim 22, wherein said antigen or antigenic determinant is selected from the group consisting of:
(a) human prion protein or a dimer thereof;
(b) bovine prion protein or a dimer thereof;
(c) sheep prion protein or a dimer thereof;
(d) elk prion protein or a dimer thereof;
(e) mule deer prion protein or a dimer thereof;
(f) white-tailed deer prion protein or a dimer thereof;
(g) pig prion protein or a dimer thereof;
(h) chicken prion protein or a dimer thereof;
(i) mouse prion protein or a dimer thereof;
(j) goat prion protein or a dimer thereof; and
(k) a peptide or fragment thereof of any prion protein of (a)-(j).

38. The composition of claim 22, wherein said antigen or antigenic determinant has an amino acid sequence selected from the group consisting of:
(a) the amino acid sequence of SEQ ID NO:79;
(b) the amino acid sequence of SEQ ID NO:80;
(c) the amino acid sequence of SEQ ID NO:81;
(d) the amino acid sequence of SEQ ID NO:82;
(e) the amino acid sequence of SEQ ID NO:83;
(f) the amino acid sequence of SEQ ID NO:84;
(g) the amino acid sequence of SEQ ID NO:85;
(h) the amino acid sequence of SEQ ID NO:86;
(i) the amino acid sequence of SEQ ID NO:87;
(j) the amino acid sequence of SEQ ID NO:88; and
(k) the amino acid sequence of a fragment of any of SEQ ID NOS:79-88.

39. The composition of claim 22, wherein said antigen or antigenic determinant is a PrP peptide.

40. The composition of claim 39, wherein said prion peptide is selected from the group consisting of:
(a) human prion peptide;
(b) bovine prion peptide;
(c) sheep prion peptide;
(d) elk prion peptide;
(e) mule deer prion peptide;
(f) white-tailed deer prion peptide;
(g) pig prion peptide;
(h) chicken prion peptide;
(i) mouse prion peptide;
(j) goat prion peptide; and
(k) a fragment of any prion peptide of (a)-(j).

41. The composition of claim 22, wherein said antigen or antigenic determinant is a PrP peptide comprising at least one antigenic site of a prion protein.

42. The composition of claim 22, wherein said antigen or antigenic determinant is a PrP peptide comprising an amino acid sequence selected from the group consisting of:
+M1 !,1 (a)? ,3 SAMSRPIIHFGSDYEDRYYRENMHR;? ,22 (SEQ ID NO: 112)? ,32 ? ! !(b)? GSDYEDRYYRENMHR;? (SEQ ID NO: 113)? ! !(c)? SAMSRPIHFGSDYEDRYYRENMHR;? (SEQ ID NO: 114)? ! !(d)? GSDYEDRYYRENMHR;? (SEQ ID NO: 115)? ! !(e)? SAMSRPLIHFGNDYEDRYYRENMYR;? (SEQ ID NO: 116)? ! !(f)? GNPYEDRYYRENMYR;? (SEQ ID NO: 117)? ! !(g)? SAMNRPLIHFGNDYEDRYYRENMYR;? (SEQ ID NO: 118)? ! !(h)? GNPYEDRYYRENMYR;? (SEQ ID NO: 119)? ! !(i)? SAMSRPLIHFGNDYEDRYYRENMYR; and? (SEQ ID NO: 120)? ! !(j)? GNDYEDRYYRENMYR.? (SEQ ID NO: 121)? !
!

43. The composition of claim 22, wherein said antigen or antigenic determinant with said at least one second attachment site comprises an amino acid sequence selected from the group consisting of:
+M1 !,1 (a)? ,3 CSAMSRPIIHFGSDYEDRYYRENMHR;? ,22 (SEQ ID NO: 122)? ,32 ? ! !(b)? CGSDYEDRYYRENMHR;? (SEQ ID NO: 123)? ! !(c)? CSAMSRPLIHFGSDYEDRYYRENMHR;? (SEQ ID NO: 124)? ! !(d)? CGSDYEDRYYRENMHR;? (SEQ ID NO: 125)? ! !(e)? CSAMSRPLIHFGNDYEDRYYRENMYR;? (SEQ ID NO: 126)? ! !(f)? CGNDYEDRYYRENMYR;? (SEQ ID NO: 127)? ! !(g)? CSAMNRPLIHFGNDYEDRYYRENMYR;? (SEQ ID NO: 128)? ! !(h)? CGNDYEDRYYRENMYR;? (SEQ ID NO: 129)? ! !(i)? CSAMSRPLIHFGNDYEDRYYRENMYR; and? (SEQ ID NO: 130)? ! !(j)? CGNDYEDRYYRENMYR.? (SEQ ID NO: 131)? !
!

44. A pharmaceutical composition comprising:
(a) the composition of claim 1; and
(b) an acceptable pharmaceutical carrier.

45. A pharmaceutical composition comprising:
(a) the composition of claim 22; and
(b) an acceptable pharmaceutical carrier.

46. A vaccine composition comprising a composition, wherein said composition comprises:
(a) a virus-like particle; and
(b) at least one antigen or antigenic determinant, wherein said antigen or said antigenic determinant is a prion protein (PrP) or a dimer thereof, or a PrP peptide, and
wherein said at least one antigen or antigenic determinant is bound to said virus-like particle.

47. The vaccine composition of claim 46, further comprising an adjuvant.

48. The vaccine composition of claim 46, wherein said virus-like particle (a) is a recombinant virus-like particle.

49. The vaccine composition of claim 46, wherein said virus-like particle is Hepatitis B virus core antigen.

50. The vaccine composition of claim 46, wherein said virus-like particle comprises recombinant proteins, or fragments thereof, of a RNA-phage.

51. The vaccine composition of claim 46, wherein said virus-like particle comprises recombinant proteins, or fragments thereof, of RNA-phage Qβ.

52. The vaccine composition of claim 46, wherein said virus-like particle comprises recombinant proteins, or fragments thereof, of RNA-phage fr.

53. The vaccine composition of claim 46, wherein said virus-like particle comprises recombinant proteins, or fragments thereof, of RNA-phage AP205.

54. The vaccine composition of claim 46, wherein said at least one antigen or antigenic determinant is bound to said virus-like particle by at least one covalent bond, and wherein said covalent bond is a non-peptide bond.

55. The vaccine composition of claim 46, wherein said at least one antigen or antigenic determinant is fused to said virus-like particle.

56. The vaccine composition of claim 46, wherein said antigen or antigenic determinant is a prion protein or a dimer thereof.

57. The vaccine composition of claim 46, wherein said antigen or antigenic determinant is selected from the group consisting of:
(a) human prion protein or a dimer thereof;
(b) bovine prion protein or a dimer thereof;
(c) sheep prion protein or a dimer thereof;
(d) elk prion protein or a dimer thereof;
(e) mule deer prion protein or a dimer thereof;
(f) white-tailed deer prion protein or a dimer thereof;
(g) pig prion protein or a dimer thereof;
(h) chicken prion protein or a dimer thereof;
(i) mouse prion protein or a dimer thereof;
(j) goat prion protein or a dimer thereof; and
(k) a peptide or fragment thereof of any prion protein of (a)-(j).

58. The vaccine composition of claim 46, wherein said antigen or antigenic determinant has an amino acid sequence selected from the group consisting of:
(a) the amino acid sequence of SEQ ID NO:79;
(b) the amino acid sequence of SEQ ID NO:80;
(c) the amino acid sequence of SEQ ID NO:81;
(d) the amino acid sequence of SEQ ID NO:82;
(e) the amino acid sequence of SEQ ID NO:83;
(f) the amino acid sequence of SEQ ID NO:84;
(g) the amino acid sequence of SEQ ID NO:85;
(h) the amino acid sequence of SEQ ID NO:86;
(i) the amino acid sequence of SEQ ID NO:87;
(j) the amino acid sequence of SEQ ID NO:88; and
(k) the amino acid sequence of a fragment of any of SEQ ID NOS:79-88.

59. The vaccine composition of claim 46, wherein said antigen or antigenic determinant is a PrP peptide.

60. The vaccine composition of claim 59, wherein said prion peptide is selected from the group consisting of:
(a) human prion peptide;
(b) bovine prion peptide;
(c) sheep prion peptide;
(d) elk prion peptide;
(e) mule deer prion peptide;
(f) white-tailed deer prion peptide;
(g) pig prion peptide;
(h) chicken prion peptide;
(i) mouse prion peptide;
(j) goat prion peptide; and
(k) a fragment of any prion peptide of (a)-(j).

61. The vaccine composition of claim 46, wherein said antigen or antigenic determinant is a PrP peptide comprising at least one antigenic site of a prion protein.

62. The vaccine composition of claim 46, wherein said antigen or antigenic determinant is a PrP peptide comprising an amino acid sequence selected from the group consisting of:
+M1 !,1 (a)? ,4 SAMSRPIIHFGSDYEDRYYRENMHR;? ,21 (SEQ ID NO:112)?,32 ? ! !(b)? GSDYEDRYYRENMHR;? (SEQ ID NO:113)? ! !(c)? SAMSRPLIHFGSDYEDRYYRENMHR;? (SEQ ID NO:114)? ! !(d)? GSDYEDRYYRENMHR;? (SEQ ID NO:115)? ! !(e)? SAMSRPLIHFGNDYEDRYYRENMYR;? (SEQ ID NO:116)? ! !(f)? GNDYEDRYYRENMYR;? (SEQ ID NO:117)? ! !(g)? SAMNRPLIHFGNDYEDRYYRENMYR;? (SEQ ID NO:118)? ! !(h)? GNDYEDRYYRENMYR;? (SEQ ID NO:119)? ! !(i)? SAMSRPLIHFGNDYEDRYYRENMYR;? (SEQ ID NO:120)? ! !(j)? GNDYEDRYYRENMYR.? (SEQ ID NO:121)? !
!

63. The vaccine composition of claim 46, wherein said antigen or antigenic determinant further comprises at least one second attachment site selected from the group consisting of:
(a) an attachment site not naturally occurring with said antigen or antigenic determinant; and
(b) an attachment site naturally occurring with said antigen or antigenic determinant.

64. The vaccine composition of claim 63, wherein said antigen or antigenic determinant with said at least one second attachment site comprises an amino acid sequence selected from the group consisting of:
+M1 !,5 (a)? +TR,8 (SEQ ID NO:122)?,32 ? !,5 CSAMSRPIIHFGSDYEDRYYRENMHR;?,32 ? ! !,5 (b)? +TR,8 (SEQ ID NO:123)?,32 ? !,5 CGSDYEDRYYRENMHR;?,32 ? ! !,5 (c)? +TR,8 (SEQ ID NO:124)?,32 ? !,5 CSAMSRPLIHFGSDYEDRYYRENMHR;? ,32 ? ! !,5 (d)? +TR,8 (SEQ ID NO:125)?,32 ? !,5 CGSDYEDRYYRENMHR;?,32 ? ! !,5 (e)? +TR,8 (SEQ ID NO:126)?,32 ? !,5 CSAMSRPLIHFGNDYEDRYYRENMYR;? ,32 ? ! !,5 (f)? +TR,8 (SEQ ID NO:127)?,32 ? !,5 CGNDYEDRYYRENMYR;? ,32 ? ! !,5 (g)? +TR,8 (SEQ ID NO:128)?,32 ? !,5 CSAMNRPLIHFGNDYEDRYYRENMYR;?,32 ? ! !,5 (h)? +TR,8 (SEQ ID NO:129)?,32 ? !,5 CGNDYEDRYYRENMYR;?,32 ? ! !,5 (i)? +TR,8 (SEQ ID NO:130)?,32 ? !,5 CSAMSRPLIHFGNDYEDRYYRENMYR;?,32 ? ! !,5 (j)? +TR,8 (SEQ ID NO:131)?,32 ? !,5 CGNDYEDRYYRENMYR.? ,32 ? !
!

65. A process for producing a composition of claim 1 comprising:
(a) providing a virus-like particle;
(b) providing at least one antigen or antigenic determinant, wherein said antigen or said antigenic determinant is a prion protein (PrP) or a dimer thereof, or a PrP peptide; and
(c) combining said virus-like particle and said at least one antigen or antigenic determinant so that said at least one antigen or antigenic determinant is bound to said virus-like particle.

66. A process for producing a composition of claim 22 comprising:
(a) providing a core particle with at least one first attachment site;
(b) providing at least one antigen or antigenic determinant with at least one second attachment site,
wherein said antigen or antigenic determinant is a prion protein (PrP) or a dimer thereof, or a prion peptide, and wherein said second attachment site is selected from the group consisting of:
(i) an attachment site not naturally occurring with said antigen or antigenic determinant; and
(ii) an attachment site naturally occurring with said antigen or antigenic determinant; and
wherein said second attachment site is capable of association to said first attachment site; and
(c) combining said core particle and said at least one antigen or antigenic determinant, wherein said antigen or antigenic determinant and said core particle interact through said association to form an ordered and repetitive antigen array.

67. A method of immunization comprising administering the composition of claim 1 to an animal or human.

68. The method of immunization of claim 67, wherein said antigen or antigenic determinant is a self-antigen.

69. The method of immunization of claim 67, wherein said animal is a human, and wherein said antigen or antigenic determinant is a human prion protein or a dimer thereof, or a human prion peptide.

70. The method of immunization of claim 67, wherein said animal is of bovine origin, and wherein said antigen or antigenic determinant is a bovine prion protein or a dimer thereof, or a bovine prion peptide.

71. The method of immunization of claim 67, wherein said animal is of sheep origin, and wherein said antigen or antigenic determinant is a sheep prion protein or a dimer thereof, or a sheep prion peptide.

72. The composition of claim 1 for use as a medicament.

73. The composition of claim 22 for use as a medicament.

74. Use of a composition of claim 1 for the manufacture of a medicament for treatment of prion diseases.

75. Use of a composition of claim 22 for the manufacture of a medicament for treatment of prion diseases.
